SEQUENCE LISTING (54 sequences)

SEQ ID NO: 1 - DNA, 1611 bp, synthetic construct
  misc_feature 1..1611: note = Synthetic polynucleotide
  misc_feature 1..1611: note = antiCD45-antiPD1 using scFv from Nivolumab modified to include Cys
  misc_feature 1..1611: note = encodes the peptide sequence at SEQ ID NO 2
  source 1..1611: mol_type = other DNA
atgctactagtaaatcagtcacaccaaggcttcaataaggaacacacaagcaagatggtaagcgctattgttttatatgtgcttttggcggcggcggcgcattctgcctttgcgggatcccaggttcagctcgtagaatctggagccgaagtcaagaaatgcggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgacctcagaggataccgctgtctactactgcgcaaggagtggcccctacgcttggttcgatacctggggccagggaactactgtcacggtatccagtggtggcggtggtagcgaaatcgtactgacccagagccctgcgactttatctttatctcctggcgaaagggctaccctgtcatgtcgcgcatctcagtccgtctcatcgtacctcgcctggtaccagcagaaacccggacaagcgcctcgtcttttgatctatgacgcctctaaccgtgccaccggtattccagctcgtttctctggctccggttccggcacagacttcactttgacgatttcttcactcgaacctgaagactttgcagtctattactgccagcagagttccaactggcctcgcaccttcggccagactaaggtggagattaaaagaagggcagacgcggccgccgctggcggtggtggaagcggtggaggaggatctggcggtggaggttccggtggaggaggaacgggtggtggaggtagtcaagttcagttggtggaatctggcggcggagtcgtacagccgggacgctcattgagactggactgtaaggctagtggaatcacattcagcaactccggaatgcactgggttaggcaagctcccggcaaaggattggaatgggtcgccgtaatttggtacgacggatctaagaggtactacgctgacagcgtttgcggtaggttcacaatctccagggataactcaaagaacacactttttctgcaaatgaactccctcaccgccgaggacaccgccgtttactactgtgcaactaacgatgattattggggtcagggtactctggtcactgtcagctccggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcatcaccaccatcaccac

SEQ ID NO: 2 - protein, 537 aa, synthetic construct
  REGION 1..537: note = Synthetic polypeptide
  REGION 1..537: note = misc_feature - antiCD45-antiPD1 using scFv from Nivolumab modified to include Cys
  REGION 1..38: note = misc_feature - Signal Peptide
  REGION 39..40: note = misc_feature - Linker
  REGION 41..158: note = misc_feature - heavy chain variable region of anti-CD45 scFv
  REGION 159..163: note = misc_feature - Linker
  REGION 164..269: note = misc_feature - light chain variable region of anti-PD1 scFv
  REGION 270..302: note = misc_feature - Linker
  REGION 303..415: note = misc_feature - heavy chain variable region of anti-PD1 scFv
  REGION 416..420: note = misc_feature - Linker
  REGION 421..527: note = misc_feature - light chain variable region of anti-CD45 scFv
  REGION 528..537: note = misc_feature - His Tag
MLLVNQSHQGFNKEHTSKMVSAIVLYVLLAAAAHSAFAGSQVQLVESGAEVKKCGASVKVSCKASGYTFTNYIIHWVKQEPGQGLEWIGYFNPYNHGTKYNEKFKGRATLTADKSISTAYMELSSLTSEDTAVYYCARSGPYAWFDTWGQGTTVTVSSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQTKVEIKRRADAAAAGGGGSGGGGSGGGGSGGGGTGGGGSQVQLVESGGGVVQPGRSLRLDCKASGITFSNSGMHWVRQAPGKGLEWVAVIWYDGSKRYYADSVCGRFTISRDNSKNTLFLQMNSLTAEDTAVYYCATNDDYWGQGTLVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHH

SEQ ID NO: 3 - DNA, 1647 bp, synthetic construct
  misc_feature 1..1647: note = Synthetic polynucleotide
  misc_feature 1..1647: note = antiCD45-antiPD1 using scFv from 1B8
  misc_feature 1..1647: note = encodes the peptide sequence at SEQ ID NO 4
  source 1..1647: mol_type = other DNA
atgctactagtaaatcagtcacaccaaggcttcaataaggaacacacaagcaagatggtaagcgctattgttttatatgtgcttttggcggcggcggcgcattctgcctttgcgggatcccaggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgagatcagaggataccgctgtctactactgcgcaaggagtggcccctacgcttggttcgatacctggggccagggaactactgtcacggtatccagtggtggcggtggtagcgacatcgtgctgacacagagccctacaagcctggccgtgtctctgggacagagagccaccatcagctgtagagccagcgagagcgtggacaacagcggcatcagcttcatgaactggttccagcagaagcccggccagcctcctaagctgctgatctacgccgccagcaatcctggaagcggagtgcctgccagattttctggcagcggctctggcaccgacttcagcctgaacattcaccccatggaagaggacgacaccgccatgtacttttgccagcagagcaaagaggtgccctggacctttggcggcggaaccgagctggaaatcaagagaagaagggcagacgcagctgccgctggcggtggtggaagcggtggaggaggttctggcggtggaggttccggtggaggtggaacgggtggaggcggtagtgaagtgcagctggttctgtctggcggcggatttgttcagcctggcggaagcctgaagctgagctgtgctgccagcggcttcacctttagcagctacgccatgagctgggtccgacagaaccctgagagaaggcttgtgtgggtcgccaccattacaggcggcggacggaacacctactatcccgattctgtgaagggcagattcaccatcagccgggacaacgccaagaacaccctgtacctgcagatgagcagcctgagaagcgaggacaccgccatgtactactgcaccagacagggctacgacggctacacttggtttgcctattggggccagggcaccctggtcacagtttctggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcatcaccaccatcaccac

SEQ ID NO: 4 - protein, 549 aa, synthetic construct
  REGION 1..549: note = Synthetic polypeptide
  REGION 1..549: note = misc_feature - antiCD45-antiPD1 using scFv from 1B8
  REGION 1..38: note = misc_feature - Signal Peptide
  REGION 39..40: note = misc_feature - Linker
  REGION 41..158: note = misc_feature - heavy chain variable region of anti-CD45 scFv
  REGION 159..163: note = misc_feature - Linker
  REGION 164..274: note = misc_feature - light chain variable region of anti-PD1 scFv
  REGION 275..308: note = misc_feature - Linker
  REGION 309..427: note = misc_feature - heavy chain variable region of anti-PD1 scFv
  REGION 428..432: note = misc_feature - Linker
  REGION 433..539: note = misc_feature - light chain variable region of anti-CD45 scFv
  REGION 540..549: note = misc_feature - His Tag
MLLVNQSHQGFNKEHTSKMVSAIVLYVLLAAAAHSAFAGSQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSDIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKRRRADAAAAGGGGSGGGGSGGGGSGGGGTGGGGSEVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHH

SEQ ID NO: 5 - DNA, 1233 bp, synthetic construct
  misc_feature 1..1233: note = Synthetic polynucleotide
  misc_feature 1..1233: note = antiCD45-IL2 fusion
  misc_feature 1..1233: note = encodes the peptide sequence at SEQ ID NO 6
  source 1..1233: mol_type = other DNA
atgaagaccatcatcgccctgagctacatcttctgcctggtgttcgccggatcccaggtgcagctggtggaatctggcgccgaagtgaaaaagcctggcgcctctgtgaaggtgtcctgcaaggccagcggctacacctttaccaactacatcatccactgggtcaagcaagagcccggccagggactcgagtggatcggctacttcaacccctacaaccacggcaccaagtacaacgagaagttcaagggcagagccacactgaccgccgacaagagcatcagcaccgcctacatggaactgagcagcctgagaagcgaggacaccgccgtgtactactgtgccagatccggaccttacgcttggtttgacacatggggccagggcaccaccgtgacagtttctagcggcggcagcagcagaagctctagttctggcggcggaggatctggcggaggcggagatattctgctgacacagagccccgccacactgtcactttctccaggcgagagagccactctgagctgtagagccagccagaacatcggcaccagcatccagtggtatcagcagaaacctggacaggcccctcggctgctgatcagatctagctccgagagcatctccggcatcagctccagattttctggcagcggctccggcaccgatttcaccctgaccataagcagcctggaacctgaggatttcgccgtgtattattgccagcagagcaacacctggcctttcacctttggccaggggaccaagctggaaatcaaaggcggcggaagctctgcccctaccagcagcagcaccaagaaaacccaactgcagctggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatctccaacatcaacgtgatcgtgctggaactgaaaggcagcgagacaaccttcatgtgcgagtacgccgacgagacagctaccatcgtggaatttctgaaccggtggatcaccttctgccagagcatcatctctaccctgacagcggccgcacaccaccatcatcaccatcaccaccat

SEQ ID NO: 6 - protein, 415 aa, synthetic construct
  REGION 1..415: note = Synthetic polypeptide
  REGION 1..415: note = misc_feature - antiCD45-IL2 fusion
  REGION 1..16: note = misc_feature - Signal Peptide
  REGION 17..18: note = misc_feature - Linker
  REGION 19..140: note = misc_feature - heavy chain variable region of anti-CD45 scFv
  REGION 141..158: note = misc_feature - Linker
  REGION 159..265: note = misc_feature - Linker
  REGION 266..270: note = misc_feature - light chain variable region of anti-CD45 scFv
  REGION 271..403: note = misc_feature - IL-2
  REGION 404..415: note = misc_feature - His Tag
MKTIIALSYIFCLVFAGSEVQLQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGSSRSSSSGGGGSGGGGDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKGGGSSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAAAHHHHHHHHH

SEQ ID NO: 7 - protein, 33 aa, synthetic construct
  REGION 1..33: note = Synthetic polypeptide
  REGION 1..33: note = misc_feature - Linker peptide
RRADAAAAGGGGSGGGGSGGGGSGGGGTGGGGS

SEQ ID NO: 8 - DNA, 25 bp, synthetic construct
  misc_feature 1..25: note = Synthetic polynucleotide
  misc_feature 1..25: note = lentiviral delivery system targeting human PD-1 sequence
  source 1..25: mol_type = other DNA
caccgcgactggccagggcgcctgt

SEQ ID NO: 9 - DNA, 1497 bp, synthetic construct
  misc_feature 1..1497: note = Synthetic polynucleotide
  misc_feature 1..1497: note = CD45-PD1 Nivo v0.1
  source 1..1497: mol_type = other DNA
caggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgagatcagaggataccgctgtctactactgcgcaaggagtggcccctacgcttggttcgatacctggggccagggaactactgtcacggtatccagtggtggcggtggtagcgaaatcgtactgacccagagccctgcgactttatctttatctcctggcgaaagggctaccctgtcatgtcgcgcatctcagtccgtctcatcgtacctcgcctggtaccagcagaaacccggacaagcgcctcgtcttttgatctatgacgcctctaaccgtgccaccggtattccagctcgtttctctggctccggttccggcacagacttcactttgacgatttcttcactcgaacctgaagactttgcagtctattactgccagcagagttccaactggcctcgcaccttcggccagactaaggtggagattaaaagaagggcagacgccgccgccgctggcggtggtggatctgggggaggaggttctggcgggggaggttccggtggaggtgggacggggggaggcggtagtcaagttcagttggtggaatctggcggcggagtcgtacagccgggacgctcattgagactggactgtaaggctagtggaatcacattcagcaactccggaatgcactgggttaggcaagctcccggcaaaggattggaatgggtcgccgtaatttggtacgacggatctaagaggtactacgctgacagcgttaagggtaggttcacaatctccagggataactcaaagaacacactttttctgcaaatgaactccctccgtgccgaggacaccgccgtttactactgtgcaactaacgatgattattggggtcagggtactctggtcactgtcagctccggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcatcaccaccatcaccaccattag

SEQ ID NO: 10 - protein, 498 aa, synthetic construct
  REGION 1..498: note = Synthetic polypeptide
  REGION 1..498: note = misc_feature - CD45-PD1 Nivo v0.1
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQTKVEIKRRADAAAAGGGGSGGGGSGGGGSGGGGTGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 11 - DNA, 1497 bp, synthetic construct
  misc_feature 1..1497: note = Synthetic polynucleotide
  misc_feature 1..1497: note = CD45-PD1 Nivo Stabilized
  source 1..1497: mol_type = other DNA
caggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgacctcagaggataccgctgtctactactgcgcaaggagtggcccctacgcttggttcgatacctggggccagggaactactgtcacggtatccagtggtggcggtggtagcgaaatcgtactgacccagagccctgcgactttatctttatctcctggcgaaagggctaccctgtcatgtcgcgcatctcagtccgtctcatcgtacctcgcctggtaccagcagaaacccggacaagcgcctcgtcttttgatctatgacgcctctaaccgtgccaccggtattccagctcgtttctctggctccggttccggcacagacttcactttgacgatttcttcactcgaacctgaagactttgcagtctattactgccagcagagttccaactggcctcgcaccttcggccagactaaggtggagattaaaagaagggcagacgcggccgccgctggcggtggtggaagcggtggaggaggatctggcggtggaggttccggtggaggaggaacgggtggtggaggtagtcaagttcagttggtggaatctggcggcggagtcgtacagccgggacgctcattgagactggactgtaaggctagtggaatcacattcagcaactccggaatgcactgggttaggcaagctcccggcaaaggattggaatgggtcgccgtaatttggtacgacggatctaagaggtactacgctgacagcgttaagggtaggttcacaatctccagggataactcaaagaacacactttttctgcaaatgaactccctcaccgccgaggacaccgccgtttactactgtgcaactaacgatgattattggggtcagggtactctggtcactgtcagctccggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcatcaccaccatcaccaccattaa

SEQ ID NO: 12 - protein, 498 aa, synthetic construct
  REGION 1..498: note = Synthetic polypeptide
  REGION 1..498: note = misc_feature - CD45-PD1 Nivo Stabilized
QVQLVESGAEVKKPGASVKVSCKASGYTFTNYIIHWVKQEPGQGLEWIGYFNPYNHGTKYNEKFKGRATLTADKSISTAYMELSSLTSEDTAVYYCARSGPYAWFDTWGQGTTVTVSSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQTKVEIKRRADAAAAGGGGSGGGGSGGGGSGGGGTGGGGSQVQLVESGGGVVQPGRSLRLDCKASGITFSNSGMHWVRQAPGKGLEWVAVIWYDGSKRYYADSVKGRFTISRDNSKNTLFLQMNSLTAEDTAVYYCATNDDYWGQGTLVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 13 - DNA, 1533 bp, synthetic construct
  misc_feature 1..1533: note = Synthetic polynucleotide
  misc_feature 1..1533: note = CD45-PD1 Pembro
  source 1..1533: mol_type = other DNA
caggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgagatcagaggataccgctgtctactactgcgcaaggagtggcccctacgcttggttcgatacctggggccagggaactactgtcacggtatccagtggtggcggtggtagcgagatcgtgctgacacagtctcccgccacactgtcactgtctccaggcgaaagagccacactgagctgcagagcctctaagggcgtgtccacaagcggctacagctacctgcactggtatcagcagaagcccggacaggctcccagactgctgatctacctggcctcctacctggaaagcggagtgcctgccagattttctggcagcggctctggcaccgatttcaccctgaccataagcagcctggaacctgaggacttcgccgtgtactactgccagcacagcagagatctgcccctgacatttggcggaggcaccaaggtggaaatcaagagaagggcagacgcggccgccgctggaggtggaggtagtggcggaggtggatctggtggaggaggctcaggtggaggcggtacgggtggcggaggtagtcaagttcaactggtgcagagcggcgtggaagtgaagaaacctggcgcctctgtgaaggtgtcctgcaaggccagcggctataccttcaccaactactacatgtactgggtccgacaggcccctggacagggacttgaatggatgggcggcatcaaccctagcaacggcggcaccaacttcaacgagaagttcaagaacagagtgaccctgacaaccgacagcagcaccaccacagcctacatggaactgaagtccctgcagttcgacgacaccgccgtgtattattgcgccagacgggactacagattcgacatgggctttgactattggggccagggcactaccgtaacagtgtcttctggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcaccaccaccatcaccaccattaa

SEQ ID NO: 14 - protein, 510 aa, synthetic construct
  REGION 1..510: note = Synthetic polypeptide
  REGION 1..510: note = misc_feature - CD45-PD1 Pembro
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRRADAAAAGGGGSGGGGSGGGGSGGGGTGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 15 - DNA, 1518 bp, synthetic construct
  misc_feature 1..1518: note = Synthetic polynucleotide
  misc_feature 1..1518: note = CD45-PD1 Cl19
  source 1..1518: mol_type = other DNA
caggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgacctcagaggataccgctgtctactactgcgcaaggagtggcccctacgcttggttcgatacctggggccagggaactactgtcacggtatccagtggtggcggtggtagcgaaaatgtgctcacccagtctccagcaatcatgtctgcatctccaggggaaaaggtcaccatgacctgcagggccagctcaagtgtaatttccagttacttgcactggtaccagcagaagtcaggtgcctcccccaaactctggatttatagcacttccaacttggcttctggagtccctgatcgattcagtggcagtgggtctgggacctcttactctctcacaatcagcagtgtggaggctgaagatgctgccacttattactgccagcagtacaatggttacccgctcacgttcggtgctgggaccaagctggaaataaaaagaagggcagacgcggccgccgctggcggtggtggaagcggtggaggaggatctggcggtggaggttccggtggaggaggaacgggtggtggaggtagtcaggttcagctacagcagtctggggctgagctggtgaagcctggggcctcagtgaagatgtcctgcaaggcttttggctacaccttcactacctatccaatagagtggatgaagcagaatcatgggaagagcctagagtggattggaaattttcatccttacaatgatgatactaagtacaatgaaaaattcaagggcaaggccaaattgactgtagaaaaatcctctaccacagtctacttggagctcagccgattaacatctgacgactctgctgtttattactgtgcaagggagaactacggtagtcacgggggttttgtttactggggccaagggactctggtcaccgtcggtggcggaggatcagacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaagcggccgcgcatcatcaccaccatcaccaccattaa

SEQ ID NO: 16 - protein, 505 aa, synthetic construct
  REGION 1..505: note = Synthetic polypeptide
  REGION 1..505: note = misc_feature - CD45-PD1 Cl19
QVQLVESGAEVKKPGASVKVSCKASGYTFTNYIIHWVKQEPGQGLEWIGYFNPYNHGTKYNEKFKGRATLTADKSISTAYMELSSLTSEDTAVYYCARSGPYAWFDTWGQGTTVTVSSGGGGSENVLTQSPAIMSASPGEKVTMTCRASSSVISSYLHWYQQKSGASPKLWIYSTSNLASGVPDRFSGSGSGTSYSLTISSVEAEDAATYYCQQYNGYPLTFGAGTKLEIKRRADAAAAGGGGSGGGGSGGGGSGGGGTGGGGSQVQLQQSGAELVKPGASVKMSCKAFGYTFTTYPIEWMKQNHGKSLEWIGNFHPYNDDTKYNEKFKGKAKLTVEKSSTTVYLELSRLTSDDSAVYYCARENYGSHGGFVYWGQGTLVTVGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 17 - DNA, 720 bp, synthetic construct
  misc_feature 1..720: note = Synthetic polynucleotide
  misc_feature 1..720: note = CD45-Db-4
  source 1..720: mol_type = other DNA
caggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatccatcagcacagcctacatggagctcagcagcctgcgctctgaggacactgcggtctactactgtgcaagatcaggaccctatgcctggtttgacacctggggccaagggaccacggtcaccgtctcctcaggaggtggcggtagtgacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatctcttccaggtttagtggcagtggatcagggacagattttactcttaccatcagcagtctggagcctgaagattttgcagtgtattactgtcaacaaagtaatacctggccattcacgttcggccaggggaccaagctggagatcaaagcggccgcgcatcatcaccaccatcaccac

SEQ ID NO: 18 - protein, 240 aa, synthetic construct
  REGION 1..240: note = Synthetic polypeptide
  REGION 1..240: note = misc_feature - CD45-Db-4
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHH

SEQ ID NO: 19 - DNA, 1170 bp, synthetic construct
  misc_feature 1..1170: note = Synthetic polynucleotide
  misc_feature 1..1170: note = CD45-PD1 VHH
  source 1..1170: mol_type = other DNA
gatgtgcagctggtggaatctggtggcggagttgttcagcctggcggctctctgagactgtcttgtgccgcctctggctctatcgcctctatccatgccatgggctggttcagacaggcccctggcaaagaaagggaattcgtggccgtgatcacttggagcggcggcatcacctactacgccgattctgtgaagggcagattcaccatcagccgggacaacagcaagaacaccgtgtacctgcagatgaacagcctgcggcctgaggacacagccctgtattattgtgccggcgacaagcaccagtcctcttggtacgattattggggccagggcaccctggtcaccgtttcttctggaggtggttctttggaagttctgtttcaagggcctggatctggatctgacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaaggcggcagcaccagaagctctagttctggtggaggaggatctggcggaggcggacaggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgagatcagaggataccgctgtctactactgcgcaaggagtggcccctacgcttggttcgatacctggggccagggaactactgtcacggtatccagtgcggccgcgcatcatcaccaccatcaccaccattaa

SEQ ID NO: 20 - protein, 389 aa, synthetic construct
  REGION 1..389: note = Synthetic polypeptide
  REGION 1..389: note = misc_feature - CD45-PD1 VHH
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGSLEVLFQGPGSGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKGGSTRSSSSGGGGSGGGGQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSAAAHHHHHHHH

SEQ ID NO: 21 - DNA, 1194 bp, synthetic construct
  misc_feature 1..1194: note = Synthetic polynucleotide
  misc_feature 1..1194: note = CD45-PD1 VHH Extended
  source 1..1194: mol_type = other DNA
gatgtgcagctggtggaatctggtggcggagttgttcagcctggcggctctctgagactgtcttgtgccgcctctggctctatcgcctctatccatgccatgggctggttcagacaggcccctggcaaagaaagggaattcgtggccgtgatcacttggagcggcggcatcacctactacgccgattctgtgaagggcagattcaccatcagccgggacaacagcaagaacaccgtgtacctgcagatgaacagcctgcggcctgaggacacagccctgtattattgtgccggcgacaagcaccagtcctcttggtacgattattggggccagggcaccctggtcaccgtttcttctggttctggttctggaggtggttctttggaagttctgtttcaagggcctggaggttcttcaggatcaggatctgacatcctgttaacacagagcccggccacgttatcgctctctccaggcgaaagggccactttaagctgcagagcttcccaaaacataggaacttcgatccaatggtaccaacaaaagccgggtcaggcaccgagactgttgatccgttccagctctgaaagcattagcggcatctcctcgaggttttcaggttccggttcaggtaccgacttcacgctgactatctcttctctggagcctgaagatttcgcagtatactactgtcagcagagcaacacttggccattcaccttcggacagggtactaagctggagatcaaaggcggcagcaccagaagctctagttctggtggaggaggatctggcggaggcggacaggttcagctcgtagaatctggagccgaagtcaagaaacctggagcatcagtaaaggttagctgcaaggcgtccggatacaccttcaccaattacatcattcattgggtgaagcaagagccgggccaaggtctggaatggatcggatactttaatccatacaaccacggaacgaaatacaacgaaaaatttaaaggcagggcaaccctcactgccgacaagtccattagtaccgcgtacatggagttgtccagtttgagatcagaggataccgctgtctactactgcgcaaggagtggcccctacgcttggttcgatacctggggccagggaactactgtcacggtatccagtgcggccgcgcatcatcaccaccatcaccaccattaa

SEQ ID NO: 22 - protein, 397 aa, synthetic construct
  REGION 1..397: note = Synthetic polypeptide
  REGION 1..397: note = misc_feature - CD45-PD1 VHH Extended
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGSGSGGGSLEVLFQGPGGSSGSGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKGGSTRSSSSGGGGSGGGGQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSAAAHHHHHHHH

SEQ ID NO: 23 - DNA, 1143 bp, synthetic construct
  misc_feature 1..1143: note = Synthetic polynucleotide
  misc_feature 1..1143: note = CD45-PD1 mRIPR-PD1
  source 1..1143: mol_type = other DNA
caggtgcaactggttgaaagcggcggaggactggtgcaccctggcgattctctgagactgtcttgtgccgccagcggcagcgtgttcaattctgccacaatgggctggtacagacagagccctggcagccagagagagctggtcgctacaatcgttgtgggcacccctacctacgccgactctgtgaagggcagattcaccatcagccgggacaacgccaagaacatcgtgtacctgcagatgaacagcctgaagcctgaggacaccgccgtgtactactgcaactacagagccacctacaccagcggctacagcagagattattggggccagggcacccaagtgaccgtgtctagcggaggtggaggcacaggcggatctgacatcgtgatgacccagtctccttccaccctgtctgcatctgtaggagacagagtcaccatcacttgccgggccagtcagggtattagtagctggttggcctggtatcagcagaaaccagggagagcccctaaggtcttgatctataaggcatctactttagaaagtggggtcccatcaaggttcagcggcagtggatctgggacagatttcactctcaccatctctagtctgcaacctgaagattttgcaacttactactgtcaacagagttacagtaccccgtggacgttcggccaggggaccaagctggaaatcaagggcggcagcaccagaagctctagttctggtggaggaggatctggcggaggcggagaggtgcagctggttcagtctgggggaggcgtggttcagcctgggaggtccctgagactctcctgtgcagcgtctggattcacctttagtagctattggatgagctgggtccgccaggctccagggaaggggctggagtgggtctcagctattagtggtagtggtggtagcacatacgcagactccgtgaagggccggttcaccatctccagagacaattccaagaacaccctgtatctgcaaatgaactcgctaagagccgaggacacggccgtatattactgtgcgaaagagaactggggatcgtacttcgatctctggggacaaggaaccacggtaaccgtatcatcagcggccgcgcatcatcaccaccatcaccaccattaa

SEQ ID NO: 24 - protein, 380 aa, synthetic construct
  REGION 1..380: note = Synthetic polypeptide
  REGION 1..380: note = misc_feature - CD45-PD1 mRIPR-PD1
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKGGSTRSSSSGGGGSGGGGEVQLVQSGGGVVQPGRSLRLSCAASGFTFSSYWMSWVRQAPGKGLEWVSAISGSGGSTYADSVKGRFTISRDNSKNTLYLQMNSLRAEDTAVYYCAKENWGSYFDLWGQGTTVTVSSAAAHHHHHHHH

SEQ ID NO: 25 - DNA, 813 bp, synthetic construct
  misc_feature 1..813: note = Synthetic polynucleotide
  misc_feature 1..813: note = CD45-CTLA4 mRIPR-CTLA4
  source 1..813: mol_type = other DNA
caggtgcagctggttgaatctggcggaggactggtgcaccctggcgattctctgagactgtcttgtgccgccagcggcagcgtgttcaattctgccacaatgggctggtacagacagagccctggcagccagagagagctggtcgctacaatcgttgtgggcacccctacctacgctgactctgtgaagggcagattcaccatcagccgggacaacgccaagaacatcgtgtacctgcagatgaacagcctgaagcctgaggacaccgccgtgtactactgcaactacagagccacctacaccagcggctacagcagagattattggggccagggcacccaagtgaccgtgtctagtggaagcgagcccaagattccccagcctcagcctaaacctcagccgcagcctttggaagttctgtttcaagggcctggatctcaggtccagcttgtggaaagcggaggtggacttgctcagcctggtggatcactgagactgagctgtgccgcttctggcagcacaatcagctctgtggccgtcggctggtataggcagacacctggaaaccagagagaatgggtcgccaccagcagcaccagctctacaacagccacatacgccgacagcgtgaaaggccggtttaccatcagcagagacaatgctaaaaacaccatctatctccagatgaactctctcaagcccgaggatacagccgtctattattgcaagaccggcctgaccaactggggacagggaacacaagtcacagtgtccagcgcggccgcgcatcatcaccaccatcaccaccattaa

SEQ ID NO: 26 - protein, 270 aa, synthetic construct
  REGION 1..270: note = Synthetic polypeptide
  REGION 1..270: note = misc_feature - CD45-CTLA4 mRIPR-CTLA4
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGSEPKIPQPQPKPQPQPLEVLFQGPGSQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGQGTQVTVSSAAAHHHHHHHH

SEQ ID NO: 27 - DNA, 1545 bp, synthetic construct
  misc_feature 1..1545: note = Synthetic polynucleotide
  misc_feature 1..1545: note = CD45-PD1_CTLA4 dRIPR-PD1_CTLA4
  source 1..1545: mol_type = other DNA
caggtccagcttgtggaaagcggaggtggacttgctcagcctggtggatcactgagactgagctgtgccgcttctggcagcacaatcagctctgtggccgtcggctggtataggcagacacctggaaaccagagagaatgggtcgccaccagcagcaccagctctacaacagccacatacgccgacagcgtgaaaggccggtttaccatcagcagagacaatgctaaaaacaccatctatctccagatgaactctctcaagcccgaggatacagccgtctattattgcaagaccggcctgaccaactggggacagggaacacaagtcacagtgtccagcggtggagggcgcgccctggtgccgcgcggcagctctggacaggtgcaactggttgaaagcggcggaggactggtgcaccctggcgattctctgagactgtcttgtgccgccagcggcagcgtgttcaattctgccacaatgggctggtacagacagagccctggcagccagagagagctggtcgctacaatcgttgtgggcacccctacctacgccgactctgtgaagggcagattcaccatcagccgggacaacgccaagaacatcgtgtacctgcagatgaacagcctgaagcctgaggacaccgccgtgtactactgcaactacagagccacctacaccagcggctacagcagagattattggggccagggcacccaagtgaccgtgtctagcggaggtggttctttggaagttctgtttcaagggcctggatctggatctgacatcgtgatgacccagtctccttccaccctgtctgcatctgtaggagacagagtcaccatcacttgccgggccagtcagggtattagtagctggttggcctggtatcagcagaaaccagggagagcccctaaggtcttgatctataaggcatctactttagaaagtggggtcccatcaaggttcagcggcagtggatctgggacagatttcactctcaccatctctagtctgcaacctgaagattttgcaacttactactgtcaacagagttacagtaccccgtggacgttcggccaggggaccaagctggaaatcaagggcggcagcaccagaagctctagttctggtggaggaggatctggcggaggcggagaggtgcagctggttcagtctgggggaggcgtggttcagcctgggaggtccctgagactctcctgtgcagcgtctggattcacctttagtagctattggatgagctgggtccgccaggctccagggaaggggctggagtgggtctcagctattagtggtagtggtggtagcacatacgcagactccgtgaagggccggttcaccatctccagagacaattccaagaacaccctgtatctgcaaatgaactcgctaagagccgaggacacggccgtatattactgtgcgaaagagaactggggatcgtacttcgatctctggggacaaggaaccacggtaaccgtatcatcagcggccgcgcatcaccaccaccatcaccaccattaa

SEQ ID NO: 28 - protein, 514 aa, synthetic construct
  REGION 1..514: note = Synthetic polypeptide
  REGION 1..514: note = misc_feature - CD45-PD1_CTLA4 dRIPR-PD1_CTLA4
QVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGQGTQVTVSSGGGRALVPRGSSGQVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGSLEVLFQGPGSGSDIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKGGSTRSSSSGGGGSGGGGEVQLVQSGGGVVQPGRSLRLSCAASGFTFSSYWMSWVRQAPGKGLEWVSAISGSGGSTYADSVKGRFTISRDNSKNTLYLQMNSLRAEDTAVYYCAKENWGSYFDLWGQGTTVTVSSAAAHHHHHHHH

SEQ ID NO: 29 - DNA, 486 bp, Homo sapiens
  misc_feature 1..486: note = Human PD-1
  source 1..486: mol_type = other DNA
cacctgcactagcggccgccggatggtttctggattctcccgatagaccctggaatcctcctacattcagccccgctctgctggtggtcacagagggcgataatgccaccttcacctgtagcttcagcaacaccagcgagagcttcgtgctgaactggtacagaatgagccccagcaaccagaccgacaagctggccgcctttcctgaggatagaagccagcctggacaggactgccggttcagagtgacacagctgcccaacggcagagacttccacatgtctgttgtgcgggccagaagaaacgacagcggcacatatctgtgcggcgccatttctctggcccctaaggctcagatcaaagagagcctgagagccgagctgagagtgaccgaaagacgggctgaagtgcccacagctcacccatctccatctccaagacctgccggccagtttcagaccctcgttggcgcgccaagtgcaggtg

SEQ ID NO: 30 - protein, 169 aa, Homo sapiens
  REGION 1..169: note = misc_feature - Human PD-1
MKTIIALSYIFCLVFAAAAPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 31 - DNA, 543 bp, Mus musculus
  misc_feature 1..543: note = Mouse PD-1
  source 1..543: mol_type = other DNA
ggatcctctggatggctgctggaagtgcctaacggcccttggagaagcctgacattctaccctgcctggctgaccgtgtctgaaggcgccaatgccaccttcacctgtagcctgagcaattggagcgaggacctgatgctgaactggaacagactgagccccagcaaccagaccgaaaagcaggccgccttctgcaacggactgtctcagcctgtgcaggacgcccggttccagatcatccagctgcctaacagacacgacttccacatgaacatcctggacacccggcggaacgacagcggcatctatctgtgtggcgccatctctctgcaccccaaggccaagatcgaggaatctcctggcgccgagctggtggtcacagagagaatcctggaaaccagcacacgctacccatctccatctccaaagcctgagggcagattccagggcatgaaggcggccgcgggtggcggtctgaacgacatcttcgaggctcagaaaatcgaatggcacgaacatcatcaccaccatcaccaccattaa

SEQ ID NO: 32 - protein, 180 aa, Mus musculus
  REGION 1..180: note = misc_feature - Mouse PD-1
GSSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMKAAAGGGLNDIFEAQKIEWHEHHHHHHHH

SEQ ID NO: 33 - DNA, 1752 bp, Homo sapiens
  misc_feature 1..1752: note = Human CD45
  source 1..1752: mol_type = other DNA
ggatcccagagccccacaccatctccaaccggactgaccacagccaagatgcctagcgtgcccctgtctagcgatcccctgcctacacacaccaccgcttttagccctgccagcaccttcgagagagagaacgacttcagcgagacaaccacctctctgagccccgacaacaccagcacacaggtgtccccagacagcctggataatgccagcgccttcaataccaccggcgtgtcctctgtgcagacccctcatctgccaacacacgccgatagccagacaccttctgccggcaccgatacacagaccttttctggctctgccgccaacgccaagctgaaccctacacctggcagcaacgccatctctgatgtgcctggcgagagaagcaccgccagcacatttccaacagaccccgtgtctcccctgaccaccacactgtctctggcccaccatagctctgctgccctgcctgccagaaccagcaacaccaccatcaccgccaataccagcgacgcctacctgaacgccagcgaaaccacaacactgagccctagcggaagcgccgtgatcagcacaaccacaatcgccaccacacctagcaagcctacctgcgacgagaagtacgccaacatcaccgtggactacctgtacaacaaagagacaaagctgttcacggccaagctcaacgtgaacgagaacgtggaatgcggcaacaatacctgcaccaacaacgaggtgcacaacctgaccgagtgcaagaacgcctccgtgtccatcagccacaacagctgcacagcccctgacaagaccctgatcctggatgtgcctcctggcgtggaaaagttccagctgcacgattgcacccaggtggaaaaggccgataccaccatctgcctgaagtggaagaacatcgagacattcacctgtgacacccagaacatcacctaccggttccagtgcggcaatatgatcttcgataacaaagaaatcaagctcgagaacctcgagcctgagcacgagtacaagtgcgacagcgagatcctgtataacaatcacaagttcaccaacgccagcaagatcatcaagaccgacttcggctctcccggcgagccccagatcatcttctgtagatctgaggccgctcaccagggcgtgatcacctggaatcctccacagcggagcttccacaacttcaccctgtgttacatcaaagaaaccgagaaggactgcctgaacctggacaagaacctcattaagtacgacctgcagaacctgaagccgtacaccaaatacgtgctgagcctgcacgcctacattatcgccaaggtgcagagaaatggcagcgccgccatgtgccacttcaccacaaaaagcgcccctccaagccaagtgtggaacatgaccgtgtctatgaccagcgacaactccatgcacgtgaagtgccggcctccacgggatagaaatggccctcacgagagataccacctggaagtcgaggccggaaataccctcgtgcggaacgagagccacaagaactgcgacttcagagtgaaggacctgcagtacagcaccgactacaccttcaaggcctacttccacaatggcgactaccctggcgagcccttcatcctgcaccacagcaccagctacaacagcaaggcggccgcgggtggcggtctgaacgacatcttcgaggctcagaaaatcgaatggcacgaacatcatcaccaccatcaccaccattaatga

SEQ ID NO: 34 - protein, 582 aa, Homo sapiens
  REGION 1..582: note = misc_feature - Human CD45
GSQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKAAAGGGLNDIFEAQKIEWHEHHHHHHHH

SEQ ID NO: 35 - DNA, 15 bp, synthetic construct
  misc_feature 1..15: note = Synthetic polynucleotide
  misc_feature 1..15: note = Linker 1 scFv-scFv
  source 1..15: mol_type = other DNA
ggtggcggtggtagc

SEQ ID NO: 36 - protein, 5 aa, synthetic construct
  REGION 1..5: note = Synthetic polypeptide
  REGION 1..5: note = misc_feature - Linker 1 scFv-scFv
GGGGS

SEQ ID NO: 37 - DNA, 99 bp, synthetic construct
  misc_feature 1..99: note = Synthetic polynucleotide
  misc_feature 1..99: note = Linker 2 scFv-scFv
  source 1..99: mol_type = other DNA
agaagggcagacgccgccgccgctggcggtggtggatctgggggaggaggttctggcgggggaggttccggtggaggtgggacggggggaggcggtagt

SEQ ID NO: 38 - protein, 33 aa, synthetic construct; identical to SEQ ID NO: 7
  REGION 1..33: note = Synthetic polypeptide
  REGION 1..33: note = misc_feature - Linker 2 scFv-scFv

SEQ ID NO: 39 - DNA, 48 bp, synthetic construct
  misc_feature 1..48: note = Synthetic polynucleotide
  misc_feature 1..48: note = Linker 3 VHH-scFv cleavable
  source 1..48: mol_type = other DNA
ggaggtggttctttggaagttctgtttcaagggcctggatctggatct

SEQ ID NO: 40 - protein, 16 aa, synthetic construct
  REGION 1..16: note = Synthetic polypeptide
  REGION 1..16: note = misc_feature - Linker 3 VHH-scFv cleavable
GGGSLEVLFQGPGSGS

SEQ ID NO: 41 - DNA, 72 bp, synthetic construct
  misc_feature 1..72: note = Synthetic polynucleotide
  misc_feature 1..72: note = Linker 4 VHH-scFv extended
  source 1..72: mol_type = other DNA
ggttctggttctggaggtggttctttggaagttctgtttcaagggcctggaggttcttcaggatcaggatct

SEQ ID NO: 42 - protein, 24 aa, synthetic construct
  REGION 1..24: note = Synthetic polypeptide
  REGION 1..24: note = misc_feature - Linker 4 VHH-scFv extended
GSGSGGGSLEVLFQGPGGSSGSGS

SEQ ID NO: 43 - DNA, 24 bp, synthetic construct
  misc_feature 1..24: note = Synthetic polynucleotide
  misc_feature 1..24: note = Linker 5 VHH-scFv non-cleavable
  source 1..24: mol_type = other DNA
ggaggtggaggcacaggcggatct

SEQ ID NO: 44 - protein, 8 aa, synthetic construct
  REGION 1..8: note = Synthetic polypeptide
  REGION 1..8: note = misc_feature - Linker 5 VHH-scFv non-cleavable
GGGGTGGS

SEQ ID NO: 45 - DNA, 54 bp, synthetic construct
  misc_feature 1..54: note = Synthetic polynucleotide
  misc_feature 1..54: note = Linker 6 scFv-scFv
  source 1..54: mol_type = other DNA
ggcggcagcaccagaagctctagttctggtggaggaggatctggcggaggcgga

SEQ ID NO: 46 - protein, 18 aa, synthetic construct
  REGION 1..18: note = Synthetic polypeptide
  REGION 1..18: note = misc_feature - Linker 6 scFv-scFv
GGSTRSSSSGGGGSGGGG

SEQ ID NO: 47 - DNA, 51 bp, synthetic construct
  misc_feature 1..51: note = Synthetic polynucleotide
  misc_feature 1..51: note = Linker 7 VHH-VHH
  source 1..51: mol_type = other DNA
ggaagcgagcccaagattccccagcctcagcctaaacctcagccgcagcct

SEQ ID NO: 48 - protein, 17 aa, synthetic construct
  REGION 1..17: note = Synthetic polypeptide
  REGION 1..17: note = misc_feature - Linker 7 VHH-VHH
GSEPKIPQPQPKPQPQP

SEQ ID NO: 49 - DNA, 39 bp, synthetic construct
  misc_feature 1..39: note = Synthetic polynucleotide
  misc_feature 1..39: note = Linker 8 VHH-VHH-scFv
  source 1..39: mol_type = other DNA
ggtggagggcgcgccctggtgccgcgcggcagctctgga

SEQ ID NO: 50 - protein, 13 aa, synthetic construct
  REGION 1..13: note = Synthetic polypeptide
  REGION 1..13: note = misc_feature - Linker 8 VHH-VHH-scFv
GGGRALVPRGSSG

SEQ ID NO: 51 - DNA, 48 bp, synthetic construct; identical to SEQ ID NO: 39
  misc_feature 1..48: note = Synthetic polynucleotide
  misc_feature 1..48: note = Linker 9 VHH-VHH-scFv
  source 1..48: mol_type = other DNA

SEQ ID NO: 52 - protein, 16 aa, synthetic construct
  REGION 1..16: note = Synthetic polypeptide
  REGION 1..16: note = misc_feature - Linker 9 VHH-VHH-scFv
GGGSLEVLFQGPGSGS

SEQ ID NO: 53 - DNA, 1206 bp, synthetic construct
  misc_feature 1..1206: note = Synthetic polynucleotide
  misc_feature 1..1206: note = CD45-CD28 mRIPR-CD28
  source 1..1206: mol_type = other DNA
caggtgcaactggttgaaagcggcggaggactggtgcaccctggcgattctctgagactgtcttgtgccgccagcggcagcgtgttcaattctgccacaatgggctggtacagacagagccctggcagccagagagagctggtcgctacaatcgttgtgggcacccctacctacgccgactctgtgaaaggcagattcaccatcagccgggacaacgccaagaacatcgtgtacctgcagatgaacagcctgaagcctgaagacaccgccgtgtactactgcaactacagagccacctacaccagcggctacagcagagattattggggccagggcacccaagtgaccgtgtctagcggaggtggttctttggaagttctgtttcaagggcctggatctggatctgacatcgtgatgacacagagccctagcagcctggccgtgtctgccggcgagaaagtgaccatgagctgcagaagcagccagagcctgtactacagcggcatcaagaagaacctgctggcctggtatcagcagaagcccggccagtctcctaagctgctgatctacttcaccagcaccagactgcctggcgtgcccgatagattcacaggatctggcagcggcaccgactacaccctgacaatcacatctgtgcaggccgaggatgagggcgactacttctgccagcaaggcatcagcacccctctgacatttggcggaggcaccaaactggagatcaaaggcggcagcaccagaagctctagttctggtggaggaggatctggcggaggcggacaggtgcagctgaaacagtctggagccgaacttgtgaaacctggcgcctctgtgaagatcagctgcaaaaccagcggctacaccttcaccgacggctacatgaactgggtcagacaggcccctggacagggcctcgagtggatcggcagaatcgatcctgatagcggcaacacccggtacaaccagaagttccagggaaaagccacactgacccgggacaagtctagcagcaccgtgtacatggacctgagaagcctgaccagcgaggacagcgccgtgtactactgtgccagagatggcaccttctacggcacctacggttactggtacttcgacttttggggtcagggcacccaagtgaccgttagctctgcggccgcgcatcaccaccaccatcaccaccattaa

SEQ ID NO: 54 - protein, 401 aa, synthetic construct
  REGION 1..401: note = Synthetic polypeptide
  REGION 1..401: note = misc_feature - CD45-CD28 mRIPR-CD28
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGSLEVLFQGPGSGSDIVMTQSPSSLAVSAGEKVTMSCRSSQSLYYSGIKKNLLAWYQQKPGQSPKLLIYFTSTRLPGVPDRFTGSGSGTDYTLTITSVQAEDEGDYFCQQGISTPLTFGGGTKLEIKGGSTRSSSSGGGGSGGGGQVQLKQSGAELVKPGASVKISCKTSGYTFTDGYMNWVRQAPGQGLEWIGRIDPDSGNTRYNQKFQGKATLTRDKSSSTVYMDLRSLTSEDSAVYYCARDGTFYGTYGYWYFDFWGQGTQVTVSSAAAHHHHHHHH

(19) United States
(12) Patent Application Publication
(10) Pub. No.: US 2026/0152556 A1
(43) Pub. Date: Jun. 4, 2026

(54) RECEPTOR INHIBITION BY PHOSPHATASE RECRUITMENT

(71) Applicant: The Board of Trustees of the Leland Stanford Junior University, Stanford, CA (US)

(72) Inventors: Kenan Christopher GARCIA, Menlo Park, CA (US); Ricardo A. FERNANDES, Oxford (GB)

(21) Appl. No.: 19/337,339

(22) Filed: Sep. 23, 2025

Related U.S. Application Data
(63) Continuation of application No. 17/055,740, now abandoned.
(60) Provisional application No. 62/673,049, filed on May 17, 2018.

(51) Int. Cl. C07K 16/28; A61K 38/00; A61K 39/00; A61K 45/06; C07K 14/715; C12N 15/113

(52) U.S. Cl. CPC C07K 16/2803; A61K 45/06; C07K 14/715; C12N 15/113; A61K 38/00; A61K 2039/505; C07K 2317/31

(57) ABSTRACT

Disclosed herein are compositions and methods for modulating cell surface receptor signaling by specifically recruiting membrane phosphatases to a proximity of receptors of interest. This novel methodology, termed Receptor Inhibition by Phosphatase Recruitment (RIPR), represents a new approach to reducing and suppressing signal by receptors that signal through phosphorylation mechanisms. More particularly, the disclosure provides novel multivalent protein-binding molecules that specifically bind a cell surface receptor and antagonize the receptor signaling through recruitment of a phosphatase activity. Also provided are compositions and methods useful for producing such molecules, as well as methods for the treatment of diseases associated with the inhibition of signal transduction mediated by cell surface receptor.

### CROSS-REFERENCE TO RELATED APPLICATIONS

The present application is a Continuation of U.S. patent application Ser. No. 17/055,740, filed on Nov. 16, 2020, which is a U.S. National Phase Application of PCT International Application No. PCT/US2019/032737, filed on May 16, 2019, designating the United States of America, which is an International Application of and claims the benefit of priority to U.S. Provisional Patent Application No. 62/673,049, filed on May 17, 2018. The disclosures of the above-referenced applications are herein expressly incorporated by reference in their entireties, including any drawings.

The invention was made with government support under grant no. CA177684 awarded by the National Institutes of Health. The government has certain rights in the present invention.

### INCORPORATION OF THE SEQUENCE LISTING

The present application is being filed along with a Sequence Listing in electronic format. The material in the accompanying Sequence Listing is hereby incorporated by reference into this application. The accompanying Sequence Listing, entitled Sequence_Listing_078430-504C01US_ST26.xml, was created on Sep. 22, 2025, and is 131,072 bytes.

### FIELD

The present disclosure relates generally to the field of immuno-therapeutics, and particularly relates to multivalent protein-binding molecules that specifically bind a cell surface receptor and antagonize the receptor signaling through recruitment of a phosphatase activity. The disclosure also provides compositions and methods useful for producing such molecules, as well as methods for the treatment of diseases associated with the inhibition of signal transduction mediated by cell surface receptors.

### BACKGROUND

Biopharmaceuticals or the use of pharmaceutical compositions comprising a therapeutic protein for the treatment of diseases or health conditions is a core strategy for a number of pharmaceutical and biotechnology companies. For example, in cancer immunotherapy, the development of agents that activate T cells of the host's immune system to prevent the proliferation of or kill cancer cells, has emerged as a promising therapeutic approach to complement existing standards of care. Examples of such immunotherapy approaches include the development of antibodies for use in modulating the immune system to kill cancer cells. For example, for antagonism of a particular activity of a receptor, the most prevalent strategy is through blockade of ligand binding between the receptor extracellular domains (ECDs) through the use of, for example, antagonist antibodies directed to the ECD of a receptor. In this scenario, the blocking molecules (e.g., the antagonist antibodies) work by competing with the natural ligand for binding to the receptor ECD. Exemplifications of this approach include a number of blocking antibodies specific for the ECDs of the immune receptors PD-1 or its ligand PD-L1 that have been approved in the US and the European Union to treat diseases such as unresectable or metastatic melanoma and metastatic non-small cell lung cancer. In another example, efforts to inhibit immunosuppressive proteins such as CTLA-4 have led to the development of commercial products and clinical evaluation of anti-CTLA-4 blocking antibodies that also work by binding to the ECD and blocking its binding to the natural ligands. However, these blocking antibodies have been reported to be ineffective in many patients, and not capable of eliminating the receptors' basal intracellular signaling activity (also referred to as resting intracellular signaling activity), such as the basal signaling activity by PD-1 and other receptors that signal through phosphorylation mechanisms. This failure to eliminate basal signaling activity frequently limits the effectiveness of ECD ligand blocking strategies. Thus, new methods are needed to directly reduce or eliminate the intracellular signaling of such receptors by alternative mechanisms other than ECD ligand blocking mechanism which would reduce or eliminate both resting and ligand-activated signaling. For example, with respect to immune checkpoint receptors, it is desirable to enable full amplification of T-cell activity by complete removal of checkpoint blockade. Additionally, new approaches are needed to inhibit signaling of other receptors, such as cytokine receptors, which signal through phosphorylation mechanisms and for which ligand blockade has demonstrated limited effectiveness.

Accordingly, there remains a need for alternative approaches other than direct receptor-ligand blockade by antibodies or other agents, to complement existing therapeutic standards of care for immunotherapy of cancer and other immune diseases.

### SUMMARY

This present disclosure relates generally to the immuno-therapeutics, such as multivalent polypeptides, multivalent antibodies, and pharmaceutical compositions comprising the same for use in treating various diseases, such as those associated with the inhibition of cell signaling mediated by a cell surface receptor. As described in greater detail below, the disclosure provides compositions and methods for modulating cell surface receptor signaling by specifically recruiting membrane phosphatases to a spatial proximity of signaling receptors of interest through, for example, direct ligation using a multivalent agent. This novel methodology is termed “Receptor Inhibition by Phosphatase Recruitment” (RIPR). More particularly, the disclosure provides novel chimeric protein-binding molecules that specifically bind a cell surface receptor, thereby completely or partially antagonizing the receptor signaling through recruitment of a phosphatase activity. In some particular embodiments, the disclosed chimeric protein-binding molecules are multivalent polypeptides. In some embodiments, the multivalent polypeptides are multivalent antibodies. The disclosure also provides compositions and methods useful for producing such compounds, as well as methods for the treatment of diseases associated with the inhibition of signal transduction mediated by cell surface receptors.

In one aspect, disclosed herein is a multivalent polypeptide which includes (i) a first amino acid sequence including a first polypeptide module capable of binding to one or more receptor protein-tyrosine phosphatases (RPTPs), and (ii) a second amino acid sequence including a second polypeptide module capable of binding to one or more cell surface receptors that signal through a phosphorylation mechanism, wherein the first polypeptide module is operably linked to the second polypeptide module.

Non-limiting exemplary embodiments of the multivalent polypeptide of the disclosure can include one or more of the following features. In some embodiments, the first polypeptide module is operably linked to the second polypeptide module via a polypeptide linker sequence. In some embodiments, at least one of the first and second polypeptide modules includes an amino acid sequence for a protein-binding ligand or an antigen-binding moiety. In some embodiments, the protein-binding ligand is a cytokine, a growth factor, a receptor extracellular domain (ECD) of a cell surface receptor or of a RPTP, or a functional variant of any thereof. In some embodiments, the antigen-binding moiety is selected from the group consisting of an antigen-binding fragment (Fab), a single-chain variable fragment (scFv), a nanobody, a V_{H} domain, a V_{L} domain, a single domain antibody (dAb), a V_{NAR} domain, and a V_{H}H domain, a diabody, or a functional fragment thereof. In some embodiments, the antigen-binding moiety includes a heavy chain variable region and a light chain variable region.

In some embodiments, the one or more RPTPs include CD45 phosphatase or a functional variant thereof. In some embodiments, the one or more cell surface receptors include an immune-checkpoint receptor, a cytokine receptor, or a growth factor receptor. In some embodiments, the one or more cell surface receptors include an immune-checkpoint receptor selected from the group consisting of inhibitory checkpoint receptors and stimulatory checkpoint receptors. In some embodiments, the one or more cell surface receptor include an inhibitory checkpoint receptor selected from the group consisting of PD-1, CTLA-4, A2AR, B7-H3, B7-H4, BTLA, CD5, CD132, IDO, KIR, LAG3, TIM-3, TIGIT, VISTA, functional variants of any thereof. In some embodiments, the one or more cell surface receptors include a stimulatory checkpoint receptor selected from the group consisting of CD27, CD28, CD40, OX40, GITR, ICOS, CD137, and functional variants of any thereof. In some embodiments, the one or more cell surface receptors mediate signaling through a specific tyrosine-based motif selected from an ITAM motif, an ITSM motif, an ITIM motif, or a related intracellular motif that serves as a substrate for phosphorylation. In some embodiments, the one or more cell surface receptors are selected from the group consisting of DAP10, DAP12, SIRPa, CD3, CD28, CD4, CD8, CD200, CD200R, ICOS, KIR, FcR, BCR, CD5, CD2, G6B, LIRs, CD7, BTNs, and functional variants of any thereof. In some embodiments, the one or more cell surface receptors include a cytokine receptor. In some embodiments, the cytokine receptor is selected from the group consisting of interleukin receptors, interferon receptors, chemokine receptors, growth hormone receptors, erythropoietin receptors (EpoRs), thymic stromal lymphopoietin receptors (TSLPRs), thrombopoetin receptors (TpoRs), granulocyte macrophage colony-stimulating factor (GM-CSF) receptors, and granulocyte colony-stimulating factor (G-CSF) receptors. In some embodiments, the one or more cell surface receptors include a growth factor receptor. In some embodiments, the growth factor receptor is a tyrosine receptor kinase (TRK) belonging to a TRK family selected from the group consisting of EGF receptor family (ErbB family), Insulin receptor family, PDGF receptor family, VEGF receptors family, FGF receptor family, CCK receptor family, NGF receptor family, HGF receptor family, Eph receptor family, AXL receptor family, TIE receptor family, RYK receptor family, DDR receptor family, RET receptor family, ROS receptor family, LTK receptor family, ROR receptor family, and MuSK receptor family. In some embodiments, the growth factor receptor is a stem cell growth factor receptor (SCFR) or an epidermal growth factor receptor (EGFR) selected from the group consisting of ErbB-1, ErbB-2 (HER2), ErbB-3, ErbB-4, and c-Kit (CD117).

In some embodiments, the polypeptide linker sequence includes 1-100 amino acid residues. In some embodiments, the polypeptide linker includes at least one glycine residue. In some embodiments, the polypeptide linker includes a glycine-serine linker. In some embodiments, the heavy chain variable region and the light chain variable region are operably linked to each other via one or more intervening amino acid residues that are positioned between the heavy chain variable region and the light chain variable region. In some embodiments, the intervening amino acid residues include 1-100 amino acid residues. In some embodiments, the intervening amino acid residues include at least one glycine residue. In some embodiments, the intervening amino acid residues include a glycine-serine linker.

Some embodiments disclosed herein relate to a multivalent polypeptide that includes, in the N-terminal to C-terminal direction, (a) a domain A including a binding region of a heavy chain variable region of a first scFv specific for an epitope of a RPTP; (b) a domain B including a binding region of a light chain variable region of a second scFv specific for an epitope of a cell surface receptor; (c) a domain C including a binding region of a heavy chain variable region of the second scFv specific for an epitope of the cell surface receptor; and (d) a domain D including a binding region of a light chain variable region of the first scFv specific for an epitope of the RPTP. In some embodiments, the multivalent polypeptide according to this aspect of the disclosure further includes an amino acid sequence for a signal peptide. In some embodiments, in some embodiments, the multivalent polypeptide according to this aspect includes an amino acid sequence that has at least 80% sequence identity to an amino acid sequence selected from the group consisting of SEQ ID NOS: 2, 4, 6, 10, 12, 14, 16, 20, 22, 24, 26, 28, and 54.

In one aspect, some embodiments disclosed herein relate to a multivalent antibody or functional fragment thereof, which includes (i) a first polypeptide module specific for one or more receptor protein-tyrosine phosphatases (RPTPs), and (ii) a second polypeptide module specific for one or more cell surface receptors that signal through a phosphorylation mechanism, wherein the first polypeptide module is operably linked to the second polypeptide module.

Non-limiting exemplary embodiments of the multivalent polypeptide of the disclosure can include one or more of the following features. In some embodiments, the first polypeptide module is operably linked to the second polypeptide module via a polypeptide linker sequence. In some embodiments, at least one of the first and second polypeptide modules includes an amino acid sequence for a protein-binding ligand or an antigen-binding moiety. In some embodiments, the antigen-binding moiety is selected from the group consisting of an antigen-binding fragment (Fab), a single-chain variable fragment (scFv), a nanobody, a V_{H} domain, a V_{L} domain, a single domain antibody (sdAb), a V_{NAR} domain, and a V_{H}H domain, or a functional fragment thereof. In some embodiments, the antigen-binding moiety includes a heavy chain variable region and a light chain variable region.

In some embodiments, the one or more RPTPs include CD45 or a functional variant thereof. In some embodiments, the one or more cell surface receptors include an immune-checkpoint receptor, a cytokine receptor, or a growth factor receptor. In some embodiments, the one or more cell surface receptors include an immune-checkpoint receptor selected from the group consisting of inhibitory checkpoint receptors and stimulatory checkpoint receptors. In some embodiments, the one or more cell surface receptors include an inhibitory checkpoint receptor selected from the group consisting of PD-1, CTLA-4, A2AR, B7-H3, B7-H4, BTLA, CD5, CD132, IDO, KIR, LAG3, TIM-3, TIGIT, VISTA, and functional variants of any thereof. In some embodiments, the one or more cell surface receptors include a stimulatory checkpoint receptor selected from the group consisting of CD27, CD28, CD40, OX40, GITR, ICOS, CD137, and functional variants of any thereof. In some embodiments, the one or more cell surface receptors mediate signaling through a specific tyrosine-based motif selected from an ITAM motif, an ITSM motif, an ITIM motif, or a related intracellular motif that serves as a substrate for phosphorylation. In some embodiments, the one or more cell surface receptors are selected from the group consisting of DAP10, DAP12, SIRPa, CD3, CD28, CD4, CD8, CD200, CD200R, ICOS, KIR, FCR, BCR, CD5, CD2, G6B, LIRs, CD7, BTNs, and functional variants of any thereof. In some other embodiments, the cell surface receptor is a cytokine receptor. In some embodiments, the cytokine receptor is selected from the group consisting of interleukin receptors, interferon receptors, chemokine receptors, growth hormone receptors, erythropoietin receptors (EpoRs), thymic stromal lymphopoietin receptors (TSLPRs), thrombopoetin receptors (TpoRs), granulocyte macrophage colony-stimulating factor (GM-CSF) receptors, granulocyte colony-stimulating factor (G-CSF) receptors. In yet some other embodiments, the cell surface receptor is a growth factor receptor. In some embodiments, the growth factor receptor is a tyrosine receptor kinase (TRK) belonging to a TRK family selected from the group consisting of EGF receptor family (ErbB family), Insulin receptor family, PDGF receptor family, VEGF receptors family, FGF receptor family, CCK receptor family, NGF receptor family, HGF receptor family, Eph receptor family, AXL receptor family, TIE receptor family, RYK receptor family, DDR receptor family, RET receptor family, ROS receptor family, LTK receptor family, ROR receptor family, and MuSK receptor family. In some embodiments, the growth factor receptor is a stem cell growth factor receptor (SCFR) or an epidermal growth factor receptor (EGFR) selected from the group consisting of ErbB-1, ErbB-2 (HER2), ErbB-3, ErbB-4, and c-Kit (CD117).

In some embodiments of the disclosure, the polypeptide linker sequence includes 1-100 amino acid residues. In some embodiments, the polypeptide linker includes at least one glycine residue. In some embodiments, the polypeptide linker includes a glycine-serine linker.

In some embodiments, the heavy chain variable region and the light chain variable region of the antigen-binding moiety are operably linked to each other via one or more intervening amino acid residues that are positioned between the heavy chain variable region and the light chain variable region. In some embodiments, the intervening amino acid residues include 1-100 amino acid residues. In some embodiments, the intervening amino acid residues include at least one glycine residue. In some embodiments, the intervening amino acid residues include a glycine-serine linker.

Some embodiments disclosed herein relate to a multivalent antibody that includes, in the N-terminal to C-terminal direction, (a) a domain A including a binding region of a heavy chain variable region of a first scFv specific for an epitope of the RPTP; (b) a domain B including a binding region of a light chain variable region of a second scFv specific for an epitope of the cell surface receptor; (c) a domain C including a binding region of a heavy chain variable region of the second scFv specific for an epitope of the cell surface receptor; and (d) a domain D including a binding region of a light chain variable region of the first scFv specific for an epitope of the RPTP. In some embodiments, the multivalent antibody according to this aspect of the disclosure further includes an amino acid sequence for a signal peptide. In some embodiments, the multivalent antibody according to this aspect includes an amino acid sequence that has at least 80% sequence identity to an amino acid sequence selected from the group consisting of SEQ ID NOS: 2, 4, 6, 10, 12, 14, 16, 20, 22, 24, 26, 28, and 54.

In another aspect, some embodiments disclosed herein relate to a pharmaceutical composition which includes (i) a multivalent polypeptide as disclosed herein; or (ii) a multivalent antibody as disclosed herein; and a pharmaceutical acceptable excipient.

In another aspects, some embodiments disclosed herein relate to a recombinant nucleic acid molecule, the nucleic acid molecule includes a nucleotide sequence encoding a polypeptide that includes (i) an amino acid sequence having at least 80% identity to the amino acid sequence of a multivalent polypeptide as disclosed herein; or (ii) an amino acid sequence having at least 80% identity to the multivalent antibody of or a functional fragment thereof as disclosed herein. In some embodiments, the nucleotide sequence has at least 80% sequence identity to a nucleotide sequence selected from the group consisting of SEQ ID NOS: 1, 3, 5, 9, 11, 13, 15, 19, 21, 23, 25, 27, and 53. In some related embodiments, the present disclosure further provides an expression cassette or a vector including a recombinant nucleic acid molecule as disclosed herein.

In another aspect, some embodiments disclosed herein relate to a recombinant cell that includes a nucleic acid molecule as disclosed herein. The recombinant cell according to this aspect includes a nucleic acid molecule including a nucleotide sequence which encodes a polypeptide including: (i) an amino acid sequence having at least 80% identity to the amino acid sequence of a multivalent polypeptide as disclosed herein; or (ii) an amino acid sequence having at least 80% identity to the multivalent antibody of or a functional fragment thereof as disclosed herein. In some embodiments, the nucleotide sequence has at least 80% sequence identity to a nucleotide sequence selected from the group consisting of SEQ ID NOS: 1, 3, 5, 9, 11, 13, 15, 19, 21, 23, 25, 27, and 53. In another related aspect, some embodiments disclosed herein relate to cell culture including one or more recombinant cells as disclosed herein.

In another aspect, disclosed herein are embodiments of methods for producing a polypeptide or a multivalent antibody that includes (i) a multivalent polypeptide as disclosed herein, or (ii) a multivalent antibody as disclosed herein. In some embodiments, the methods according to this aspect are performed in vitro, in vivo, or ex vivo.

In another aspect, disclosed herein are embodiments of methods for modulating cell signaling mediated by a cell surface receptor that signals through a phosphorylation mechanism in a subject, the method including administering to the subject a first therapy including an effective amount of (i) a multivalent polypeptide as disclosed herein, or (ii) a multivalent antibody as disclosed herein.

In yet another aspect, disclosed herein are embodiments of methods for the treatment of a disease in a subject in need thereof, the method including administering to the subject a first therapy including an effective amount of (i) a multivalent polypeptide as disclosed herein, or (ii) a multivalent antibody as disclosed herein.

Non-limiting exemplary embodiments of the embodiments of the methods of the disclosure can include one or more of the following features. In some embodiments, the administered multivalent polypeptide or the multivalent antibody recruits the receptor protein-tyrosine phosphatase (RPTP) activity to a spatial proximity of the cell surface receptor and reduces phosphorylation level of the cell surface receptor. In some embodiments, the administration of the multivalent polypeptide or the multivalent antibody confers a reduced activity of an immune checkpoint receptor in the subject. In some embodiments, the administration of the multivalent polypeptide or the multivalent antibody confers an enhancement in T-cell activity in the subject. In some embodiments, the administration of the multivalent polypeptide or the multivalent antibody confers suppression of T-cell activity in the subject. In some embodiments, the subject is a mammal. In some embodiments, the mammal is human. In some embodiments, the subject has or is suspected of having a disease associated with inhibition of cell signaling mediated by the cell surface receptor. In some particular embodiments, the disease is a cancer or a chronic infection.

In some embodiments, the disclosed treatment methods further include administering to the subject a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapeutic agent and the second therapy are administered together in a single formulation.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B schematically illustrate a non-limiting example of the modulation of cell surface receptor signaling by local phosphatase recruitment through the RIPR method, in accordance with some embodiments of the disclosure. Active kinases at the cell membrane induce low, basal, levels of receptor phosphorylation (FIG. 1A-left panel). Binding to cognate ligands increases receptor phosphorylation and initiates signaling (FIG. 1A-right panel). As illustrated, a bispecific polypeptide that recruits phosphatases to the spatial proximity of receptors of interest reduces both basal as well as ligand-induced phosphorylation (FIG. 1B, by enzymatically ‘shaving’ the phosphates from the receptor intracellular domain in both its basal and ligand-activated states (FIG. 1B). The receptor-binding module of the RIPR molecule may be either competitive or non-competitive with the natural ligand, which can either be secreted or membrane bound.

FIG. 2 schematically illustrates a non-limiting example of the application of the RIPR method to the modulation of PD-1 surface receptor signaling by local CD45 recruitment in accordance with some embodiments of the disclosure. PD-1 expression reduces T-cell activity due to the low, basal, phosphorylation of the intracellular motif by membrane-bound kinases, such as Lck (FIG. 2—top; left panel). Upon binding to PD-L1, PD-1 phosphorylation is increased, which further decreases T-cell activity (FIG. 2—top; right panel). PD-1 blocking antibodies, “checkpoint inhibitors” impair receptor/ligand interaction and thus reduce PD-L1-induced phosphorylation. As illustrated, a bispecific diabody that recruits the CD45 phosphatase to the spatial proximity of receptors of interest reduces both basal as well as PD-L1-induced phosphorylation (FIG. 2—bottom left panel), removing the phosphates from the receptor's intracellular signaling motif (FIG. 2—bottom right panel).

FIGS. 3A-3C graphically summarize the results from experiments performed to illustrate that a bispecific diabody targeting human CD45 and PD-1 bound to HEK293 cells transfected with CD45 (FIG. 3A), PD-1 (FIG. 3B) or both molecules (FIG. 3C).

FIGS. 4A-4B graphically summarize the results from experiments performed to illustrate that PD-1 expression reduces T-cell activation, even in the absence of PD-1 ligands. In these experiments, Jurkat T cells expressing PD-1 were activated with OKT3 at 2 μg/ml overnight. FIG. 4A: CD25 and CD69 up-regulation was lower for cells expressing PD-1. FIG. 4B: Reduced PD-1 expression in cells treated with CRISPR/Cas9 PD-1 targeted guide RNA lead to higher CD69 expression upon activation with OKT3.

FIGS. 5A-5B summarize the results from experiments performed to illustrate a reconstitution of PD-1 phosphorylation by Lck and CD45 in HEK293 cells. PD-1 was not phosphorylated in wild-type HEK293 cells (lane 1). However, PD-1 was readily phosphorylated when Lck is also present (lane 2). Co-expression of CD45 (lane 3) reduced overall phosphorylation. Upon incubation with a CD45-PD1 bispecific diabody (Db), a further reduction in PD-1 phosphorylation was observed (lane 4). A CD45 mutant (C856S) mutation with severely reduced phosphatase activity did not affect PD-1 phosphorylation either upon expression (lane 5) or recruitment after incubation with a CD45-PD1 bispecific diabody (Db; lane 6).

FIGS. 6A-6B summarize the results from experiments performed to illustrate a reconstitution of multiple receptor phosphorylation by incubation with the lymphocyte-specific protein tyrosine kinase Lck and/or CD45 in HEK293 cells.

FIGS. 7A-7F summarize the results from experiments performed to illustrate that treatment of T cells with CD45-PD1 bispecific diabody increases T-cell activation in response to OKT3 and peptide-MHC stimulation. Jurkat T cells expressing PD-1 were stimulated overnight with OKT3 (2 μg/ml; solid diamond) in the presence of nivolumab antibody (open square) or CD45-PD1 (Nivo) diabody (closed circle). CD45-PD1 increased the expression of the activation markers CD69 (FIG. 7A) and CD25 (FIGS. 7B-7C) as well as IL-2 cytokine secretion (FIG. 7D). In FIGS. 7E-7F, SKW-3 T cells transduced with appropriate TCR and PD-1 were incubated with cells presenting agonist peptide-MHC±PD-L1 (PD-L1-, solid diamond; PD-L1+, open circle) and nivolumab antibody (open square) or CD45-PD1 (Nivo) diabody (closed circle). Incubation with CD45-PD1 diabody increased IL-2 cytokine secretion to levels similar to those achieved when PD-L1 is absent.

FIGS. 8A-8B summarize the results from experiments performed to illustrate that CD45-PD1 diabody potentiates proliferation of activated peripheral blood mononuclear cells (PBMCs). FIG. 8A: Freshly isolated PBMCs were labeled with CFSE and incubated with OKT3 plus nivolumab or CD45-PD1 diabody for 4 days. CD45-PD1 potentiated T-cell proliferation at higher levels than the nivolumab antibody. FIG. 8B: Quantification of the percentage of proliferation for T cells for cells treated with OKT3 alone or in combination with OKT3 and nivolumab or CD45-PD1 (Nivo) (0.5 μM).

FIGS. 9A-9F summarize the results from another experiment performed with activated PBMCs to illustrate that a bispecific diabody CD45-PD1 can potentiate CD4+ and CD8+ T-cell activation in response to agonist peptides. It was observed that both CD45-PD1 (Nivo) and CD45-PD1 (Pembro) could potentiate T-cell activation as indicated by elevated expression levels of CD69 (FIG. 9A) and CD25 (FIG. 9B), as well as secretion of IFNγ (FIG. 9D) and cytokine IL-2 (FIG. 9C). Upon incubation with nivolumab, pembrolizumab, CD45-PD1 (Nivo) or CD45-PD1 (Pembro), staining with anti-PD1 fluorescently labelled antibody (clone 29F.1A12; PD-1/PD-L1 blocking antibody) was diminished, as indicated by PD1 MFI, while incubation with agonist peptides alone or in combination with anti-CD45 bispecific diabody was not affected (FIG. 9E). Elevated expression levels of CD69 upon treatment with CD45-PD1 (Nivo) or CD45-PD1 (Pembro) were observed for both CD4+ and CD8+ T cells (FIG. 9F).

FIGS. 10A-10C summarize the results of another experiment performed with activated PBMCs to illustrate that RIPR-PD1 is not strictly dependent on PD-1/PD-L1 interaction blockade. It was observed that a bispecific diabody CD45-PD1 (C119), using a non-blocking scFv to bind to PD-1 (Clone 19; C119) could potentiate T-cell activation in response to agonist peptides as indicated by elevated expression levels of CD69 (FIG. 10A) as well as secretion of IFNγ (FIG. 10B). Incubation with the bispecific diabody CD45-PD1 (C119) could lead to a partial decrease of PD1 MFI after staining cells with the fluorescently labelled anti-PD1 antibody (clone 29F.1A12; PD-1/PD-L1 blocking antibody) while incubation with nivolumab or pembrolizumab could lead to a stronger reduction in PD-1 MFI (FIG. 10C).

FIGS. 11A-11C summarize the results of experiments performed to illustrate that experiments performed to illustrate that treatment of T cells with a second generation CD45-PD1 (VHH) bispecific binding module increases T-cell activation in response to Muromonab-CD3® (OKT3). In these experiments, the bispecific diabody CD45-PD1 (Nivo) and CD45-PD1 (VHH) increased the expression of the activation markers CD69 (FIG. 11A) and CD25 (FIG. 11B) resulting in a higher fraction of CD69+/CD25+ cells (FIG. 11C).

FIGS. 12A-12B summarize the results of experiments performed to illustrate that treatment of mouse T cells with an anti-mouse CD45 (VHH)-PDIF2 bispecific binding module increases T-cell activation in response to anti mouse-CD3 (2C11). In these experiments, the bispecific diabody CD45 (VHH)-PDIF2 increased the expression of the activation markers CD69 (FIG. 12A) and CD25 (FIG. 12B).

FIGS. 13A-13B summarize the results of experiments performed to illustrate that treatment of mouse TCR transgenic (Pmel-1) CD8+ T cells with an anti-mouse CD45 (VHH)-PD1 (F2) bispecific binding module increases T-cell activation in response to gp 100 peptide. In these experiments, the bispecific CD45 (VHH)-PD1F2 binding molecule increased the expression of the activation markers CD69 (FIG. 13A) and CD25 (FIG. 13B).

FIGS. 14A-14B summarize the results of experiments performed to illustrate that treatment of mouse T cells with an anti-mouse CD45 (VHH)-CTLA4 bispecific binding module, designated mRIPR-CTLA4, increases T-cell activation in response to anti mouse-CD3 (2C11). In these experiments, treatment of T cells with the bispecific CD45 (VHH)-CTLA4 binding molecule increased the fraction of cells with elevated levels of CD69 and CD25 for both CD4+ and CD8+ T cells after incubation with 2C11 antibody and CD45 (VHH)-CTLA4 for 24 hours (FIG. 14A) and 48 hours (FIG. 14B).

FIGS. 15A-15B summarize the results of experiments demonstrating that a mRIPR-CD28 reduces the expression of markers of T-cell activation, such as CD25 and CD44, in response to anti mouse-CD3 (2C11). In these experiments, an anti-mouse CD45 (VHH)-CD28 bispecific polypeptide reduces the expression of the activation markers CD25 and CD44, for both CD4+ (FIG. 15A) and CD8+ (FIG. 15B) T cells after incubation with 2C11 antibody and mRIPR-CD28 for 48 hours.

FIGS. 16A-16C schematically illustrate another non-limiting example of a bispecific protein-binding molecule in accordance with some embodiments of the disclosure. In this case, the drawing shows an example of a RIPR composed of a CD45-binding module linked to IL-2. IL-2 induces phosphorylation of its IL-2R-beta and gamma-c receptors. Linkage of IL-2 to a binding module that recruits CD45 results in the removal of phosphates from tyrosine residues on the IL-2 receptors, resulting in reduced signaling. A similar RIPR design is expected to reduce signaling by other cytokine and growth factor receptors. (FIG. 16A). A multivalent polypeptide capable of binding to phosphatase CD45 and the cell surface receptor IL-2R fluorescently labels the YT+ cell surface (FIG. 16B). Recruitment of CD45 to the IL-2 receptor decreases phosphorylation of STAT5 (pSTAT5; FIG. 16C).

FIGS. 17A-17B summarize the results of experiments performed to characterize a trispecific RIPR design in accordance with some embodiments of the disclosure. In these experiments, an anti-mouse trispecific CD45-PD1-CTLA4 was designed and constructed with an anti-mouse CD45 VHH fused to an anti-mouse PD1 scFv and further fused to an anti-mouse CTLA-4 VHH. The resulting trispecific RIPR molecule was designated double RIPR (dRIPR)-PD1/CTLA4). The amino acid sequence of this dRIPR-PD1/CTLA4 molecule is set forth in SEQ ID NO: 28 of the Sequence Listing. FIG. 17A: Protein purity after size-exclusion chromatography (AKTA FPLC, GE Healthcare, Superdex 200 Increase; 280 nm absorbance is shown) FIG. 17B: Protein purity and integrity were confirmed by non-reducing SDS-PAGE electrophoresis followed by standard Coomassic staining.

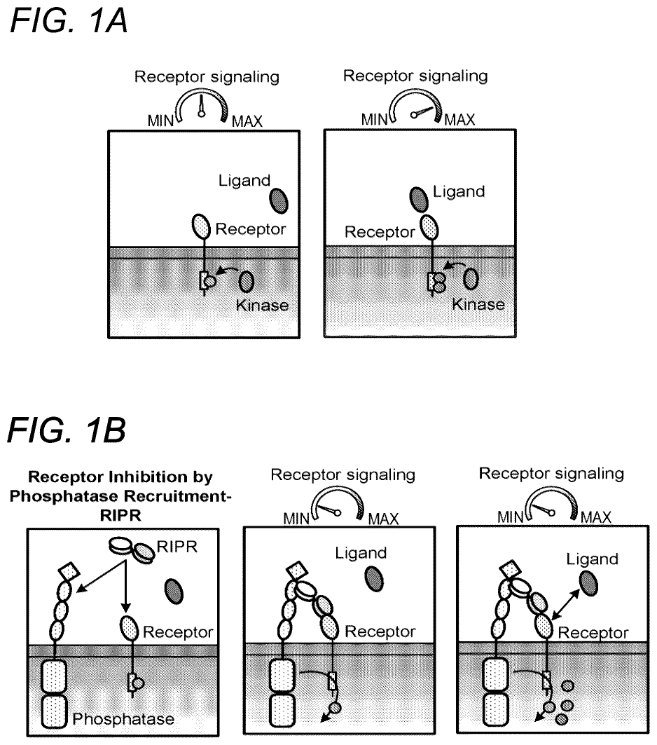

### DETAILED DESCRIPTION OF THE DISCLOSURE

The present disclosure relates generally to the field of molecular biology immunology, and medicine, including compositions and methods for a novel method, termed RIPR, of modulating cell surface receptor signaling by specifically recruiting membrane phosphatases to the spatial proximity of receptors of interest. This method for inhibiting receptor signaling represents an alternative approach to ECD ligand blockade, and thus a new paradigm for receptor antagonism in general. More particularly, the disclosure provides novel chimeric protein-binding molecules that specifically bind a cell surface receptor and antagonize the receptor's signaling, cither completely or partially, through recruitment of a phosphatase activity. In some embodiments, the recruitment of phosphatase is achieved via physical ligation. In some embodiments of the disclosure, the chimeric protein-binding molecules are multivalent polypeptides (e.g., bivalent or trivalent) including a first polypeptide fragment capable of binding to a receptor protein-tyrosine phosphatase (RPTP), and a second polypeptide fragment capable of binding to a cell surface receptor that signals through a phosphorylation mechanism. The disclosure also relates to compositions and methods useful for producing such multivalent (e.g., bispecific) protein-binding molecules, as well as methods for the treatment of diseases associated with the inhibition of signal transduction mediated by cell surface receptors.

As described in greater detail below, the present disclosure provides for, inter alia, engineered multivalent polypeptides, each exhibiting binding affinity to at least two cellular targets: a receptor protein-tyrosine phosphatase (RPTP) and cell surface receptor that signals through a phosphorylation mechanism. Without being bound by any particular theory, it is believed that the multivalent polypeptide recruits the phosphatase activity encoded by RPTP to the spatial proximity of the cell surface receptor, subsequently reduces its phosphorylation. It is also believe that the multivalent molecule facilitates the modulation of the activity of a cell surface receptor that signals through a phosphorylation mechanism by binding to the extracellular domain of the cell surface receptor and the extracellular domain of a transmembrane phosphatase such that the intracellular domains of the cell surface receptor and phosphatase are brought into sufficiently close proximity such that intracellular domain of the phosphatase dephosphorylates the intracellular domain of the cell surface receptor (or associated phosphorylated molecules) thereby reducing the activity of the cell surface receptor. In the case of checkpoint receptor and where the RPTP is CD45, ligation of a module which binds to the extracellular domain of the checkpoint receptor to a module which binds to the extracellular domain of the receptor protein-tyrosine phosphatase CD45 results in potentiation of T-cell signaling. It is also believed that, without being bound by any particular theory, reducing the activity of “immune checkpoint” receptors is expected to enhance T-cell activity and is useful as a therapy for a wide range of diseases, including cancer and chronic infection. This novel approach bypasses the current traditional strategy of regulating cellular receptor function through ligand blockade to regulating cellular receptor function by dephosphorylation of the receptor intracellular domain(s).

It has been recognized that the current clinical options to modulate cell surface receptors is limited to ECD blocking antibodies, which block a receptor-ligand interaction from occurring at the surface of the cell. For example, in the case of inhibitory receptors such as PD-1, blocking the extracellular PD-1/PD-L1 interaction with high affinity antibodies has, to date, been the only available means to reduce PD-1 signaling. However, antibody blocking does not directly affect PD-1 phosphorylation and, importantly, does not reverse the basal, tonic, phosphorylation of PD-1. As described in greater detail below, the inventors have shown that even in the absence of PD-L1, PD-1 decreases T-cell activation by nearly 50%. Without being bound by any particular theory, it is believed that existing blocking antibodies are not capable of completely eliminating PD-1 basal signaling in order to recover full T-cell activity, as determined by higher levels of the T-cell activation markers CD69 and CD25, as well as higher levels of IFNγ and IL-2 cytokine release. As described in some embodiments of the present disclosure, newly engineered multivalent antibodies address this problem by directly recruiting a phosphatase to dephosphorylate PD-1. Here, the present disclosure shows that CD45 recruitment is able to eliminate the exhausted phenotype induced by PD-1, in the presence or absence of PD-1 ligands (e.g., PD-L1). Accordingly, recruitment of phosphatases, and in particular of CD45, to receptors of interest represents a novel way to modulate the activity of cell surface receptors of interest.

The approaches disclosed herein represent several advantages. The concept of recruiting a phosphatase activity to targets of interest is very modular and versatile, and in principle can be easily adapted to target a variety of receptors. For example, the target phosphatase can be chosen from the group of surface phosphatases expressed in cells of interest (Alonso et al., 2004; Neel and Tonks, 1997). In addition, multiple receptors that signal via tyrosine phosphorylation could be targeted in a similar manner. Non-limiting examples of suitable receptors include growth factor receptors, cytokine receptors, and other checkpoint inhibitors. Furthermore, the degree of receptor inhibition can also be tuned, from complete inhibition to partial inhibition, by ways of varying the orientation and spatial proximity of the binding modules within the multivalent polypeptides (also referred hereafter as “RIPR molecules”) of the disclosure.

### General Experimental Procedures

The practice of the present disclosure will employ, unless otherwise indicated, conventional techniques of molecular biology, microbiology, cell biology, biochemistry, nucleic acid chemistry, and immunology, which are known to those skilled in the art. Such techniques are explained in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Beaucage et al. eds., Current Protocols in Nucleic Acid Chemistry, John Wiley & Sons, Inc., New York, 2000, (including supplements through 2014), Gene Transfer and. Expression in Mammalian Cells (Makrides, ed., Elsevier Sciences B.V., Amsterdam, 2003), and Current Protocols in Immunology (Horgan K and S. Shaw (1994) (including supplements through 2014). As appropriate, procedures involving the use of commercially available kits and reagents are generally carried out in accordance with manufacturer defined protocols and/or parameters unless otherwise noted.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

As used herein, the term “antibody” refers to a class of proteins that are generally known as immunoglobulins that specifically bind to, and is thereby defined as complementary with, a particular spatial and polar organization of another molecule. The term antibody includes full-length monoclonal antibodies (mAb), such as IgG2 monoclonal antibodies, which include immunoglobulin Fc regions. The term antibody also includes multivalent antibodies, diabodies, single-chain antibodies, single chain variable fragments (scFvs), and antibody fragments such as Fab, F(ab′)2, and Fv. In instances where the antibody is a multivalent antibody, the multivalent antibody can be in many different formats. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. As such, antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal subject, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

As used herein, the term “chimeric” polypeptide refers to a polypeptide comprising at least two amino acid sequences operably linked with each other, with which they are not naturally linked in nature. The amino acid sequences may normally exist in separate proteins that are brought together in the chimeric polypeptide or they may normally exist in the same protein but are placed in a new arrangement in the chimeric polypeptide. A chimeric polypeptide may be created, for example, by chemical synthesis, or by creating and translating a polynucleotide in which the peptide regions are encoded in the desired relationship.

The terms, “cells”, “cell cultures”, “cell line”, “recombinant host cells”, “recipient cells” and “host cells” as used herein, include the primary subject cells and any progeny thereof, without regard to the number of transfers. It should be understood that not all progeny are exactly identical to the parental cell (due to deliberate or inadvertent mutations or differences in environment); however, such altered progeny are included in these terms, so long as the progeny retain the same functionality as that of the originally transformed cell.

As used herein, the term “construct” is intended to mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, e.g., operably linked.

The term “effective amount,” “therapeutically effective amount,” or “pharmaceutically effective amount” of a subject multivalent polypeptide or multivalent antibody of the disclosure generally refers to an amount sufficient for a composition to accomplish a stated purpose relative to the absence of the composition (e.g., achieve the effect for which it is administered, treat a disease, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “functional fragment thereof” or “functional variant thereof” relates to a molecule having qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For example an antibody capable of binding to an epitope of a cell surface receptor may be truncated at the N-terminus and/or C-terminus, and the retention of its epitope binding activity assessed using assays known to those of skill in the art, including the exemplary assays provided herein. When referencing a polypeptide having an enzymatic activity (e.g., an enzyme such as a receptor protein-tyrosine phosphatase; RPTP), the term “functional variant” refers to an enzyme that has a polypeptide sequence that is at least about 70%, at least about 75%, at least about 80%, at least about 85%, at least about 90%, at least about 95% or at least about 99% identical to a polypeptide sequence encoding the enzyme. The “functional variant” enzyme may retain amino acids residues that are recognized as conserved for the enzyme, and may have non-conserved amino acid residues substituted or found to be of a different amino acid, or amino acid(s) inserted or deleted, but which does not affect or has insignificant effect its enzymatic activity, as compared to the enzyme described herein. The “functional variant” enzyme has an enzymatic activity that is identical or essentially identical to the biological activity of the enzyme (e.g., RPTP) described herein. One skilled in the art will appreciate that the “functional variant” enzyme may be found in nature, i.e. naturally occurring, or be an engineered mutant thereof.

The term “operably linked”, as used herein, denotes a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. In addition, in the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, modules, or domains of the multivalent polypeptides or multivalent antibodies of the disclosure may be operably linked to retain proper folding, processing, targeting, expression, binding, and other functional properties of the multivalent polypeptides or multivalent antibodies in the cell. Unless stated otherwise, various modules, domains, and segments of the multivalent polypeptides or multivalent antibodies of the disclosure are operably linked to each other. Operably linked modules, domains, and segments of the multivalent polypeptides or multivalent antibodies of the disclosure may be contiguous or non-contiguous (e.g., linked to one another through a linker).

The terms “percent identity”, in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity typically exists over a region that is at least about 20 amino acids or nucleotides in length, or over a region that is 10-100 amino acids or nucleotides in length, or over the entire length of a given sequence.

If necessary, sequence identity can be calculated using published techniques and widely available computer programs, such as the GCS program package (Devereux et al, Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

The term “recombinant” or “engineered” nucleic acid molecule or polypeptide as used herein, refers to a nucleic acid molecule or polypeptide that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is a multivalent polypeptide or bispecific antigen-binding polypeptide as disclosed herein.

A “signal peptide” or “signal sequence” is targeting sequence constituted by an amino acid sequence which, when operably linked to a terminus of a polypeptide, e.g., its N-terminus, directs the translocation thereof into the endoplasmic reticulum (ER) in a eukaryotic host cell.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

As used herein, the term “VHH” refers to variable domain of a heavy-chain antibody. As used herein, the terms “VH” and “VL” refer to the variable heavy and variable light chains of conventional antibodies, respectively.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments. As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

### Cell Surface Receptors

Cell surface receptors, often called transmembrane receptors, are proteins that mediate communication between the cell and the outside world. These receptors are responsible for the binding of an extracellular signaling molecule and transduction of its messages into one or more intracellular signaling molecules, which changes the cell's behavior. Cell surface receptors are intrinsically embedded in the plasma membrane. These receptors acts as enzymes or associate with enzymes inside the cell. When stimulated, the enzyme activate a variety of intracellular signaling pathways. They were discovered through their role in responses to extracellular signal proteins that regulates the growth, proliferation, differentiation and survival of cells in animal tissues. Diseases of cell growth, proliferation, differentiation, survival and migration are fundamental to cancer, and abnormalities in signaling via enzyme-coupled receptors have a major role in the development of this class of diseases.

Cell surface receptors act in cell signaling by receiving (binding to) extracellular molecules. The extracellular molecules may be hormones, neurotransmitters, cytokines, growth factors, cell adhesion molecules, or nutrients; they react with the receptor to induce changes in the metabolism and activity of a cell. In the process of cell signaling, signal transduction processes through membrane receptors involve the external reactions, in which the ligand binds to a membrane receptor, and the internal reactions, in which intracellular response is triggered.

Because of the important nature of these pathways, mutations in cell surface receptors are responsible for a wide array of diseases, including autoimmunity, cancers, neurodegeneration, achondroplasia and atherosclerosis. In fact, nearly half of all drugs in clinical use target cell surface receptors, and these proteins and their ligands remain extremely important targets for structure-based drug design and novel drug development. Cell surface receptors are divided into three major classes: (i) ion channel-linked receptors, (ii) enzyme-linked receptors, and (iii) G protein-coupled receptors. Of those, enzyme-linked receptors are usually single-pass transmembrane receptors which directly linked to intracellular enzymes. This class includes the extensively studied receptor tyrosine kinases (RTKs) and receptors that signal though Janus Kinases (JAKS) and STATs, the latter known as JAK/STAT cytokine receptor, which bind to polypeptide growth factors that control cell proliferation and differentiation. As described in greater detail below, RPTP recruitment is a method applicable to kinase-linked receptors that signal through a phosphorylation mechanism, which principally applies to ITAM/ITIM-containing receptors and related immune receptors (Bezbradica et al., 2012), JAK/STAT cytokine receptors (Rawlings et al., 2004), and RTK receptors that can be active in both ligand-dependent and independent states (Bergeron et al., 2016).

The largest family of enzyme-linked receptors are the receptor protein-tyrosine kinases, which phosphorylate their substrate proteins on tyrosine residues. This family includes the receptors for most polypeptide growth factors, therefore protein-tyrosine phosphorylation has been particularly well studied as a signaling mechanism involved in the control of animal cell growth and differentiation. Indeed, the first protein-tyrosine kinase was discovered in 1980 during studies of the oncogenic proteins of animal tumor viruses, in particular Rous sarcoma virus. The epidermal growth factor (EGF) receptor, which was then found to function as a protein-tyrosine kinase clearly established protein-tyrosine phosphorylation as a key signaling mechanism in the response of cells to growth factor stimulation.

Currently, more than 50 receptor protein-tyrosine kinases have been identified, including the receptors for epidermal growth factor (EGF), nerve growth factor (NGF), platelet-derived growth factor (PDGF), insulin, and many other growth factors. All these receptors share a common structural organization: an N-terminal extracellular ligand-binding domain, a single transmembrane α helix, and a cytosolic C-terminal domain with protein-tyrosine kinase activity. The majority of the receptor protein-tyrosine kinases consist of single polypeptides, although the insulin receptor and some related receptors are dimers consisting of two pairs of polypeptide chains. The binding of ligands (e.g., growth factors) to the extracellular domains of these receptors activates their cytosolic kinase domains, resulting in phosphorylation of both the receptors themselves and intracellular target proteins that propagate the signal initiated by growth factor binding. The first step in signaling from most receptor protein-tyrosine kinases is ligand-induced receptor dimerization. Some growth factors, such as PDGF and NGF, are themselves dimers consisting of two identical polypeptide chains; these growth factors directly induce dimerization by simultaneously binding to two different receptor molecules. Other growth factors (such as EGF) are monomers but have two distinct receptor binding sites that serve to crosslink receptors.

Ligand-induced dimerization then leads to autophosphorylation of the receptor as the dimerized polypeptide chains cross-phosphorylate one another. Such autophosphorylation plays two important roles in signaling from these receptors. First, phosphorylation of tyrosine residues within the catalytic domain may play a regulatory role by increasing receptor protein kinase activity. Second, phosphorylation of tyrosine residues outside of the catalytic domain creates specific binding sites for additional proteins that transmit intracellular signals downstream of the activated receptors. The association of these downstream signaling molecules with receptor protein-tyrosine kinases is mediated by protein domains that bind to specific phosphotyrosine-containing peptides. The best-characterized of these domains are called SH2 domains (for Src homology 2) because they were first recognized in protein-tyrosine kinases related to Src, the oncogenic protein of Rous sarcoma virus. SH2 domains consist of approximately one hundred amino acids and bind to specific short peptide sequences containing phosphotyrosine residues. The resulting association of SH2-containing proteins with activated receptor protein-tyrosine kinases can have several effects: It localizes the SH2-containing proteins to the plasma membrane, leads to their association with other proteins, promotes their phosphorylation, and stimulates their enzymatic activities. The association of these proteins with autophosphorylated receptors thus represents the first step in the intracellular transmission of signals initiated by the binding of growth factors to the cell surface.

Another family of enzyme-linked receptors are cytokine receptors and nonreceptor protein-tyrosine kinases (also called cytokine receptor superfamily). Rather than possessing intrinsic enzymatic activity, many receptors act by stimulating intracellular protein-tyrosine kinases (e.g., JAK/TYK) with which they are noncovalently associated. This family of receptors includes the receptors for most cytokines (e.g., interleukin-2 and erythropoietin) and for some polypeptide hormones (e.g., growth hormone). Like receptor protein-tyrosine kinases, the cytokine receptors contain N-terminal extracellular ligand-binding domains, single transmembrane α helices, and C-terminal cytosolic domains. However, the cytosolic domains of the cytokine receptors are devoid of any known catalytic activity. Instead, the cytokine receptors function in association with nonreceptor protein-tyrosine kinases, which are activated as a result of ligand binding.

The first step in signaling from cytokine receptors is believed to be ligand-induced receptor dimerization and cross-phosphorylation of the associated nonreceptor protein-tyrosine kinases. These activated kinases then phosphorylate the receptor, providing phosphotyrosine-binding sites for the recruitment of downstream signaling molecules that contain SH2 domains. Combinations of cytokine receptors plus associated nonreceptor protein-tyrosine kinases thus function analogously to the family of receptor protein-tyrosine kinases.

The nonreceptor protein-tyrosine kinases associated with the cytokine receptors fall into two major families. Many of these kinases are members of the Src family, which consists of Src and eight closely related proteins. Src was initially identified as the oncogenic protein of Rous sarcoma virus and was the first protein shown to possess protein-tyrosine kinase activity, therefore it has played a pivotal role in experiments leading to our current understanding of cell signaling. In addition to Src family members, the cytokine receptors are associated with nonreceptor protein-tyrosine kinases belonging to the Janus kinase, or JAK, family. Members of the JAK family appear to be universally required for signaling from cytokine receptors, indicating that JAK family kinases play a critical role in coupling these receptors to the tyrosine phosphorylation of intracellular targets. In contrast, members of the Src family play key roles in signaling from antigen receptors on B and T lymphocytes but do not appear to be required for signaling from most cytokine receptors.

Although the majority of enzyme-linked receptors stimulate protein-tyrosine phosphorylation, some receptors are associated with other enzymatic activities. These receptors include protein-tyrosine phosphatases. Protein-tyrosine phosphatases remove phosphate groups from phosphotyrosine residues, thus acting to counterbalance the effects of protein-tyrosine kinases. In many cases, protein-tyrosine phosphatases play negative regulatory roles in cell signaling pathways by terminating the signals initiated by protein-tyrosine phosphorylation. However, some protein-tyrosine phosphatases are cell surface receptors whose enzymatic activities play a positive role in cell signaling. An example is provided by phosphatase CD45, which is expressed on the surface of T and B lymphocytes. Following antigen stimulation, CD45 is believed to dephosphorylate a specific phosphotyrosine that inhibits the enzymatic activity of Src family members. Thus, the CD45 protein-tyrosine phosphatase acts to stimulate nonreceptor protein-tyrosine kinases.

Several members of cell surface receptors are regulators of immune system, e.g., immune checkpoints, which can be stimulatory checkpoints or inhibitory checkpoints. These receptors do not possess intrinsic enzymatic activity, but instead act as substrates for kinases via their intracellular ITAM, ITSM, and/or ITIM motifs (see, e.g., Pardoll, 2012). These receptors activity is also controlled through a balance of phosphorylation and phosphatase activities that act on their intracellular domains, and are thus good candidates for signal modulation by phosphatase ligation as described. Of those, inhibitory checkpoints have been increasingly considered as attractive targets for cancer immunotherapy due to their potential for use in multiple types of cancers (Topalian et al., 2015). Currently approved checkpoint inhibitors block CTLA-4 and PD-1 and PD-L1. Another two stimulatory checkpoint molecules belong to the B7-CD28 superfamily-CD28 itself and ICOS. Inhibitory checkpoints include, but are not limited to, PD-1, CTLA-4, A2AR, B7-H3, B7-H4, BTLA, CD5, CD132, IDO, KIR, LAG3, TIM-3, TIGIT, and VISTA, and functional variants thereof.

### PD-1

PD-1, also known as Programmed Cell Death Protein 1 and CD279 (cluster of differentiation 279), is a cell surface receptor that plays an important role in down-regulating the immune system and promoting self-tolerance by suppressing T-cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells). It is believed that through these mechanisms, PD-1 inhibits the immune system. This prevents autoimmune diseases, but it can also prevent the immune system from killing cancer cells. The PD-1 protein in humans is encoded by the PDCD1 gene.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including P815 mastocytoma, PAI myeloma, and B16 melanoma upon treatment with IFN-γ. PD-L2 expression is more restricted and is expressed mainly by DCs and a number of tumor lines.

### CTLA-4

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA-4 acts as an “off” switch when bound to CD80 or CD86 on the surface of antigen-presenting cells. The CTLA-4 protein is encoded by the Ctla4 gene in mice and the CTLA4 gene in humans. Variants in this gene have been associated with insulin-dependent diabetes mellitus, Graves' disease, Hashimoto's thyroiditis, celiac disease, systemic lupus erythematosus, Graves' disease, Hashimoto's thyroiditis, celiac disease, thyroid-associated orbitopathy, primary biliary cirrhosis and other autoimmune diseases. Polymorphisms of the CTLA-4 gene are associated with autoimmune diseases such as autoimmune thyroid disease and multiple sclerosis, though this association is often weak. In Systemic Lupus Erythematosus (SLE), a splice variant of CTLA-4 is found to be aberrantly produced and found in the serum of patients with active SLE.

CTLA-4 is expressed by activated T cells and transmits an inhibitory signal to T cells. CTLA-4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. However, CTLA-4 transmits an inhibitory signal to T cells, whereas CD28 transmits a stimulatory signal. CTLA-4 is also found in regulatory T cells and contributes to its inhibitory function.

### CD28

CD28 (Cluster of Differentiation 28) is one of the proteins expressed on T cells that provide co-stimulatory signals required for T-cell activation and survival. T-cell stimulation through CD28 in addition to the T-cell receptor (TCR) is reported to provide a potent signal for the production of various interleukins (IL-6 in particular). The co-stimulatory receptor CD28 is activated by its ligands, B7.1 (CD80) and B7.2 (CD86), and couples with TCR signaling to promote T-cell proliferation and survival during T-cell priming. When activated by Toll-like receptor ligands, the CD80 expression is upregulated in antigen-presenting cells (APCs). The CD86 expression on antigen-presenting cells is constitutive (expression is independent of environmental factors). CD28 is known to be a B7 receptor constitutively expressed on naive T cells. Inhibition of Cd28^{-/-} MRL-Ipr in murine lupus models have been shown to exhibit delayed and diminished glomerulonephritis and an absence of renal vasculitis and arthritis, implying that blocking CD28-B7 interactions might be a potential treatment for autoimmune lupus.

### TIM-3

TIM-3 (T-cell immunoglobulin and mucin-domain containing-3), which belongs to TIM family cell surface receptor proteins, is a transmembrane receptor protein that is expressed, e.g., on Th1 (T helper 1) CD4+ cells and cytotoxic CD8+ T cells that secrete IFN-γ. TIM-3 is generally not expressed on naïve T cells but rather upregulated on activated, effector T cells. TIM-3 has a role in regulating immunity and tolerance in vivo.

In human, TIM-3 is encoded by the HAVCR2 gene, which was first described as a cell surface molecule expressed on IFNγ producing CD4+ Th1 and CD8+ Tc1 cells. The expression of TIM-3 was subsequently detected in Th 17 cells, regulatory T-cells, and innate immune cells, such as, e.g., dendritic cells, NK cells, monocytes. TIM-3 contains five conserved tyrosine-residues that is believed to interact with multiple components of T-cell receptor (TCR) complex and negatively regulates its function. TIM-3 is considered to be an immune checkpoint and together with other inhibitory receptors including programmed cell death protein 1 (PD-1) and lymphocyte activation gene 3 protein (LAG3) mediate the CD8+ T-cell exhaustion. TIM-3 has also been shown as a CD4+ Th1-specific cell surface protein that regulates macrophage activation and enhances the severity of experimental autoimmune encephalomyelitis in mice.

### CD5

CD5 is a cluster of differentiation expressed on the surface of T cells in various species and in a subset of murine B cells known as B-1a. CD5 is a type I glycoprotein and a member of the scavenger-receptor family. CD5 is expressed by thymocytes, mature T cells and a subset of mature B cells and has been shown to be involved in modulation of lymphocyte activation and in the differentiation process. CD72, gp80-40 and Ig framework structures are purposed ligands for CD5 and their interaction with CD5 have been shown in mice. CD5 has been used as a T-cell marker until monoclonal antibodies against CD3 were developed. It has been reported that CD5, which may be homophilic, can bind on the surface of other cells. T cells express higher levels of CD5 than B cells. CD5 is upregulated on T cells upon strong activation. In the thymus, there is a correlation with CD5 expression and strength of the interaction of the T cell towards self-peptides.

CD5 is associated with CD79a and CD79b transduction partner of surface IgM in the vicinity of the B-cell receptor (BCR) and CD5 signaling is mediated by co-precipitation with the BCR and CD79a and CD79b into lipid rafts. CD79a and CD79b are phosphorylated by the Lyn and other tyrosine kinases such as Syk, and Zap70 as well as the tyrosine phosphatase SHP-1 have been reported to be mediators of this signal transduction.

### CD132

CD132 (common gamma chain—γc), also known as interleukin-2 receptor subunit gamma or IL-2RG, is a cytokine receptor subunit that is common to the receptor complexes for several different interleukin receptors, including IL-2, IL-4, IL-7, IL-9, IL-15 and interleukin-21 receptor. The γc chain partners with these ligand-specific receptors to direct lymphocytes to respond to cytokines. The γc glycoprotein is a member of the type I cytokine receptor family expressed on most lymphocyte (white blood cell) populations. In human, CD132 is encoded by the IL2RG gene.

CD132 is expressed on the surface of immature blood-forming cells in bone marrow. One end of the CD132 protein resides outside the cell where it binds to cytokines and the other end of the protein resides in the interior of the cell where it transmits signals to the cell's nucleus. CD132 partners with other proteins to direct blood-forming cells to form lymphocytes. Lymphocytes expressing CD132 can form functional receptors for these cytokine proteins, which transmit signals from one cell to another and direct programs of cellular differentiation.

### TIGIT

TIGIT (T-cell immunoreceptor with Ig and ITIM domains), a member of the immunoglobulin superfamily with an immunoreceptor tyrosine-based inhibitory motif (ITIM) in the cytoplasmic tail, is expressed on subsets of activated T cells and natural killer (NK) cells. Other names for TIGIT include WUCAM and Vstm3. TIGIT is known to interact with CD155 (i.e., PVR or necl-5), CD112 (PVRL2 or nectin-2), and possibly CD113 (PVRL3 or nectin-3). Binding of TIGIT with a high affinity ligand CD155, which are expressed on antigen-presenting cells, has been reported to suppress the function of T cells and NK cells. TIGIT has also been reported to inhibit T cells indirectly by modulating cytokine production by dendritic cells. It has been reported that TIGIT-Fc fusion protein could interact with PVR on dendritic cells and increase its IL-10 secretion level/decrease its IL-12 secretion level under LPS stimulation, and also inhibit T cell activation in vivo. TIGIT's inhibition of NK cytotoxicity can be blocked by antibodies against its interaction with CD155 and the activity is directed through its ITIM domain.

### Receptor Type Protein Tyrosine Phosphatases (RPTPS)

Reversible protein tyrosine phosphorylation is a major mechanism regulating cellular signaling that affects fundamental cellular events including metabolism, proliferation, adhesion, differentiation, migration, communication, and adhesion. For example, protein tyrosine phosphorylation determines protein functions, including protein-protein interactions, conformation, stability, enzymatic activity and cellular localization. Disruption of this key regulatory mechanism contributes to a variety of human diseases including cancer, diabetes, and auto-immune diseases. Net protein tyrosine phosphorylation is determined by the dynamic balance of the activity of protein tyrosine kinases (PTKs) and protein tyrosine phosphatases (PTPs). Aberrant regulation of the delicate balance between PTKs and PTPs is involved in the pathogenesis of a number of human diseases such as cancer, diabetes, and autoimmune diseases.

PTPs constitute a large and structurally diverse family of enzymes. Sequencing data indicate that there are 107 PTP genes in the human genome, of which 81 encode active protein phosphatases. Among the PTP super family, 38 are classical, tyrosine-specific PTPs, while the other 43 are dual-specificity tyrosine/serine, threonine phosphatases. The classical PTPs possess at least one catalytic domain known as the PTP domain. The 280-amino acid PTP catalytic domain contains an invariable active site signature motif (I/V) HCXAGXXR (S/T) G, which includes an essential cysteine that catalyzes nucleophilic attack on the phosphoryl group of its substrate and subsequent substrate dephosphorylation.

The PTPs can be further sub-divided into transmembrane receptor-like PTPs (RPTPs) and non-transmembrane PTPs based on their overall structure. Of these, receptor-type protein tyrosine phosphatases (RPTPs) are a family of integral cell surface proteins that possess intracellular PTP activity, and extracellular domains (ECDs) that have sequence homology to cell adhesion molecules (CAMs). Intracellular domains (ICDs) of most of the RPTPs contain two tandem PTP domains, termed D1 and D2. Generally, membrane proximal PTP domain (D1) possesses most of the catalytic activity, whereas membrane-distal PTP domain (D2) has weak, if any, catalytic activity. The ECDs of RPTPs contain combinations of CAM-like motifs with sequences homologous to fibronectin type III (FN3), meprin, A5, PTPμ (MAM), immunoglobulin (Ig), and carbonic anhydrase (CA). Collectively, the molecular structure of RPTPs enables direct coupling of extracellular adhesion-mediated events to regulation of intracellular signaling pathways.

Based on the structure of their ECDs, the RPTP family can be grouped into eight sub-families: R1/R6, R2A, R2B, R3, R4, R5, R7, and R8. Representative members of these sub-families include CD45, LAR, RPTP-κ, DEP1, RPTP-α, RPTP-ζ, PTPRR, and IA2, respectively. Further information regarding the structural features that define each of the sub-families, their molecular/biochemical structure, mode of regulation, substrate specificity, and biological functions has been extensively documented and can be found in, e.g., Xu Y. et al. (J. Cell Commun. Signal. 6:125, 138, 2012).

### CD45

The receptor type protein tyrosine phosphatase CD45, also called the leukocyte common antigen (LCA), is the sole member of the R1/R6 subtype of RPTPs. CD45 is a type I transmembrane protein that is in various forms present on all differentiated hematopoietic cells, except erythrocytes and plasma cells, and assists in the activation of those cells (a form of co-stimulation). CD45 is expressed in lymphomas, B-cell chronic lymphocytic leukemia, hairy cell leukemia, and acute nonlymphocytic leukemia. Human CD45, which is encoded by the gene PTPRC, is a cell membrane tyrosine phosphatase expressed by all cells of lymphoid origin, including hematopoietic cells, with the exception of platelets and erythrocytes, and functions as a key regulator of T and B cell signaling. CD45 consists of an extracellular region, short transmembrane segment and tandem PTP domains in the cytoplasmic region. Multiple isoforms of CD45 are generated by complex alternative splicing of exons in the extracellular domain of the molecule, which are expressed in a cell type specific manner depending on the cell differentiation and activation status. Non-limiting examples of CD45 isoforms include CD45RA, CD45RB, CD45RC, CD45RAB, CD45RAC, CD45RBC, CD45R0, CD45R (ABC). CD45RA is located on naive T cells and CD45R0 is located on memory T cells. CD45R is the longest protein and migrates at 200 kDa when isolated from T cells. B cells also express CD45R with heavier glycosylation, bringing the molecular weight to 220 kDa, hence the name B220; B cell isoform of 220 kDa. B220 expression is not restricted to B cells and can also be expressed on activated T cells, on a subset of dendritic cells and other antigen-presenting cells. Naive T lymphocytes express large CD45 isoforms and are usually positive for CD45RA. Activated and memory T lymphocytes express the shortest CD45 isoform, CD45R0, which lacks RA, RB, and RC exons. This shortest isoform is believed to facilitate T-cell activation.

CD45 plays important roles in immune system development and function and is required for antigen-specific lymphocyte stimulation and proliferation. CD45 regulates immune responses by controlling the TCR activation threshold, modulating cytokine responses, and regulating lymphocyte survival. All of these processes are essential in the pathogenesis of autoimmune and infectious diseases.

CD45 is a suitable RPTP target for being recruited to many immune receptors, because it will act on a broad range of substrates if they are brought into a spatial proximity of one to another, e.g. the two RPTP-binding and receptor-binding modules are in sufficient proximity to achieve dephosphorylation of the intracellular domain of the receptor. CD45 mediates T- and B-cell receptor function by regulating tyrosine phosphorylation of the Src family of PTKs (SFKs) like Lyn and Lck. CD45 dephosphorylates the inhibitory C-terminal phosphorylation site in Lyn and Lck, thereby potentiating the activity of these SFKs. Attenuation of SFK activity by CD45-mediated dephosphorylation of other tyrosines has also been reported. Studies in CD45 knockout mice show that CD45-mediated activation of Fyn and Lck is important in thymocyte development. Upon TCR ligation, activated Fyn and Lck phosphorylate components of the TCR complex like TCR-zeta and CD3-epsilon. These tyrosine-phosphorylated proteins provide docking sites for Src-homology 2 (SH2) domain-containing proteins to transmit down-stream signal. In CD45-null thymocytes, ligation of TCR does not lead to Lyn or Lck activation or to subsequent tyrosine phosphorylation of the TCR complex. Therefore, none of the down-stream signaling events occur; indicating the essential role of CD45 in TCR activation. CD45 has also been identified as a PTP that dephosphorylates the CD3-zeta and CD3-epsilon ITAMs, Janus kinases (JAKs) and negatively regulates cytokine receptor activation.

### Compositions of the Disclosure

### Multivalent Polypeptides and Multivalent Antibodies

In one aspect, some embodiments disclosed herein relate to a novel chimeric polypeptides containing multiple polypeptide modules, e.g., modular protein-binding moieties, each capable of binding to one or more target protein(s). In some embodiments, the disclosed chimeric polypeptide includes (i) a first amino acid sequence including a first polypeptide module capable of binding to a receptor protein-tyrosine phosphatase (RPTP), and (ii) a second amino acid sequence including a second polypeptide module capable of binding to a cell surface receptor that signals through a phosphorylation mechanism, wherein the first polypeptide module is operably linked to the second polypeptide module. In some embodiment, the disclosed chimeric polypeptide is a multivalent polypeptide. In some embodiment, the multivalent polypeptide is a multivalent antibody. The binding of a first polypeptide module and a second polypeptide module to their respective target can be either in a competitive or non-competitive fashion with a natural ligand of the target. Accordingly, in some embodiments of the disclosure, the binding of a first polypeptide module and/or second polypeptide module to their respective target can be ligand-blocking. In some other embodiments, the binding of a first polypeptide module and/or second polypeptide module to their respective target does not block binding of the natural ligand. As used herein, the term “multivalent polypeptide” as used herein refers to a polypeptide comprising two or more protein-binding modules that are operably linked to each other. For example, a “bivalent” polypeptide of the disclosure comprises two protein-binding modules, whereas a “trivalent” polypeptide of the disclosure comprises three protein-binding modules. The amino acid sequences of the polypeptide modules may normally exist in separate proteins that are brought together in the multivalent polypeptide or they may normally exist in the same protein but are placed in a new arrangement in the multivalent polypeptide. A multivalent polypeptide may be created, for example, by chemical synthesis, or by creating and translating a polynucleotide in which the peptide regions are encoded in the desired relationship.

Designation of the amino acid sequence of the chimeric polypeptide, e.g., multivalent polypeptide that includes a first polypeptide module capable of binding to a receptor protein-tyrosine phosphatase (RPTP) as the “first” amino acid sequence and the amino acid sequence of the multivalent polypeptide including a polypeptide module capable of binding to a cell surface receptor as the “second” amino acid sequence is not intended to imply any particular structural arrangement of the “first” and “second” amino acid sequences within the multivalent polypeptide. By way of non-limiting example, in some embodiments of the disclosure, the multivalent polypeptide or multivalent antibody may include an N-terminal polypeptide module capable of binding to a RPTP and a C-terminal polypeptide module including a polypeptide capable of binding to a cell surface receptor. In other embodiments, the multivalent polypeptide or multivalent antibody may include an N-terminal polypeptide module capable of binding to a cell surface receptor and a C-terminal polypeptide module capable of binding to a RPTP. In addition or alternatively, the multivalent polypeptide or multivalent antibody may include more than one polypeptide module (e.g., module) capable of binding to a RPTP, and/or more than one polypeptide module capable of binding to a cell surface receptor. Accordingly, in some embodiments, a first amino acid sequence of the multivalent polypeptide or multivalent antibody includes at least two, three, four, five, six, seven, eight, nine, or ten polypeptide modules each capable of binding to a RPTP. In some embodiments, the at least two, three, four, five, six, seven, eight, nine, or ten polypeptide modules of a first amino acid sequence are each capable of binding to the same RPTP. In some embodiments, the at least two, three, four, five, six, seven, eight, nine, or ten polypeptide modules of a first amino acid sequence are each capable of binding to different RPTPs.

In some embodiments, the second amino acid sequence of the multivalent polypeptide or multivalent antibody includes at least two, three, four, five, six, seven, eight, nine, or ten polypeptide modules each capable of binding to a cell surface receptor. In some embodiments, the at least two, three, four, five, six, seven, eight, nine, or ten polypeptide modules of the second amino acid sequence are each capable of binding to the same cell surface receptor. In some embodiments, the at least two, three, four, five, six, seven, eight, nine, or ten polypeptide modules of the second amino acid sequence are each capable of binding to different cell surface receptors. A non-limiting example of such multivalent polypeptides or multivalent antibodies containing multiple polypeptide modules each capable of binding to different cell surface receptors is described in Example 17.

In addition or alternatively, as alluded to above, the multivalent polypeptides and antibodies as disclosed herein can incorporate both natural and unnatural amino acids at positions that affect the binding affinity of the multivalent polypeptides or multivalent antibodies with the respective target protein(s). As such, the binding affinity of the polypeptide modules to their respective target (e.g., RPTP or cell surface receptor) can be tuned to achieve a desired target cell specificity. For example, since CD45 is widely expressed, the PD1-binding module can be configured to form a high affinity binding module, while the CD45-binding module can be configured to have lower binding affinity. For instance, in some embodiments, a cell-surface receptor-binding module has a higher affinity (lower K_{d}) to the cell-surface receptor when compared to the binding affinity of the RPTP-binding module to the RPTP. In some embodiments, the difference in affinity is at least one order of magnitude or at least two orders of magnitude (e.g., the ratio of the K_{d} for the interaction of the RPTP-binding module to the RPTP to the K_{d} for the interaction of the cell-surface receptor binding module to the cell-surface receptor is at least 10, at least 20, at least 50, or at least 100). One skilled in the art will appreciate that this concept of a multivalent polypeptide or multivalent antibody having high affinity for the RPTP or its target receptor, and lower affinity for the other can be an important part of tuning RIPR activity for target cell specificity. Accordingly, in some embodiments, the binding affinity of the RPTP-binding polypeptide module can be different from the binding affinity of the receptor-binding polypeptide module. For example, in some embodiments, the RPTP-binding polypeptide module has high affinity to its target and the receptor-binding polypeptide module has low affinity to its target. In some embodiments, the RPTP-binding polypeptide module has low affinity to its target and the cell surface receptor-binding polypeptide module has high affinity to its target. In some embodiments, the RPTP-binding and receptor-binding modules have the same affinity to the respective target proteins.

In some embodiments, the binding affinity of the receptor-binding and RPTP-binding modules each having an affinity for the extracellular domain of its respective target, is independently from K_{d}=10⁻⁵ to 10⁻¹² M, such as e.g., a K_{d} of about 10⁻⁵ to about 10⁻¹¹ M, alternatively a K_{d} of about 10⁻⁵ to about 10⁻¹⁰ M, alternatively a K_{d} of about 10⁻⁶ to about 10⁻¹² M, alternatively a K_{d} of about 10⁻⁷ to about 10⁻¹² M, alternatively a K_{d} of about 10⁻⁸ to about 10⁻¹² M, alternatively a K_{d} of about 10⁻⁹ to about 10⁻¹² M, alternatively a K_{d} of about 10⁻¹⁰ to about 10⁻¹² M, alternatively a K_{d} of about 10⁻¹¹ to about 10⁻¹² M, alternatively a K_{d} of about 10⁻⁵ to about 10⁻¹¹ M, alternatively a K_{d} of about 10⁻⁵ to about 10⁻¹⁰ M, alternatively a K_{d} of about 10⁻⁵ to about 10⁻⁹ M, alternatively a K_{d} of about 10⁻⁵ to about 10⁻⁸ M, alternatively a K_{d} of about 10⁻⁵ to about 10⁻⁷ M, alternatively a K_{d} of about 10⁻⁵ to about 10⁻⁶ M.

In some embodiments, the multivalent polypeptide or multivalent antibody as disclosed herein has a binding affinity for a RPTP (e.g., CD45) with a K_{d} of about 1,000 nM, about 800 nM, about 700 nM, about 600 nM, about 500 nM, about 400 nM, about 200 nM, about 100 nM, about 10 nM, about 5 nM, or about 1 nM. In some embodiments, the multivalent polypeptide or multivalent antibody as disclosed herein have low binding affinity for a RPTP, e.g. with a K_{d} of more than about 10⁻⁵ M, such as e.g., a K_{d} of more than about 10⁻⁴ M, more than about 10⁻³ M, more than about 10⁻² M, or more than about 10⁻¹ M. In some embodiments, the binding affinity (K_{d}) for a RPTP (e.g., CD45) can be about 700 nM. In some embodiments, the binding affinity of the multivalent polypeptide or multivalent antibody for CD45 can be about 300 nM.

In some embodiments, the multivalent polypeptide or multivalent antibody as disclosed herein can have binding affinity for a cell surface receptor (e.g., PD-1) with a K_{d} of 1,000 nM, about 800 nM, about 700 nM, about 600 nM, about 500 nM, about 400 nM, about 200 nM, about 150 nM, about 100 nM, about 80 nM, about 60 nM, about 40 nM, about 20 nM, about 10 nM, about 5 nM, or about 1 nM. In some embodiments, the multivalent polypeptide or multivalent antibody as disclosed herein has a high binding affinity for a cell surface receptor, e.g. with a K_{d} of less than about 10⁻⁸ M, less than about 10⁻⁹ M, less than about 10⁻¹⁰ M, less than about 10⁻¹¹ M, or less than about 10⁻¹² M. In some embodiments, the affinity for a cell surface receptor can be about 7 nM. In some embodiments, the binding affinity of the multivalent polypeptide or multivalent antibody for a cell surface receptor can be about 6 nM. In some embodiments, the binding affinity for a cell surface receptor can be about 5 nM.

In some embodiments, a first amino acid sequence of the multivalent polypeptide or multivalent antibody is directly linked to a second amino acid sequence. In some embodiments, a first amino acid sequence is directly linked to a second amino acid sequence via at least one covalent bond. In some embodiments, a first amino acid sequence is directly linked to a second amino acid sequence via at least one peptide bond. In some embodiments, the C-terminal amino acid of a first amino acid sequence can be operably linked to the N-terminal amino acid of a second polypeptide module. Alternatively, the N-terminal amino acid of a first polypeptide module can be operably linked to the C-terminal amino acid of a second polypeptide module.

In some embodiments, a first amino acid sequence of the multivalent polypeptide or multivalent antibody is operably linked to a second amino acid sequence via a linker. There is no particular limitation on the linkers that can be used in the multivalent polypeptides described herein. In some embodiments, the linker is a synthetic compound linker such as, for example, a chemical cross-linking agent. Non-limiting examples of suitable cross-linking agents that are commercially available include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES). Other examples of alterative structures and linkages suitable for the multivalent polypeptides and multivalent antibodies of the disclosure include those described in Spiess et al., Mol. Immunol. 67:95-106, 2015.

In some embodiments, a first amino acid sequence of a multivalent polypeptide or multivalent antibody disclosed herein is operably linked to a second amino acid sequence via a linker polypeptide sequence (peptidal linkage). In principle, there are no particular limitations to the length and/or amino acid composition of the linker polypeptide sequence. In some embodiments, any arbitrary single-chain peptide comprising about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues.

In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool to achieve a tuning effect that would enhance or reduce the RPTP activity of the multivalent polypeptide. In some embodiments, the orientation and/or proximity of a first and a second polypeptide modules relative to one another can be optimized to create a partial antagonist to full antagonist versions of the bispecific polypeptide. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser) n, wherein n is an integer of one or more; and (Ser Gly Gly Gly Gly) n, wherein n is an integer of one or more. In some embodiments, the linker polypeptides are modified such that the amino acid sequence Gly Ser Gly (GAG) (that occurs at the junction of traditional Gly/Ser linker polypeptide repeats) is not present. For example, in some embodiments, the polypeptide linker includes an amino acid sequence selected from the group consisting of: (GGGXX)nGGGGS and GGGGS(XGGGS)n, where X is any amino acid that can be inserted into the sequence and not result in a polypeptide comprising the sequence GSG, and n is 0 to 4. In some embodiments, the sequence of a linker polypeptide is (GGGX1X2)nGGGGS and X1 is P and X2 is S and n is 0 to 4. In some embodiments, the sequence of a linker polypeptide is (GGGX1X2)nGGGGS and X1 is G and X2 is Q and n is 0 to 4. In some other embodiments, the sequence of a linker polypeptide is (GGGX1X2)nGGGGS and X1 is G and X2 is A and n is 0 to 4. In some embodiments, the sequence of a linker polypeptide is GGGGS(XGGGS)n, and X is P and n is 0 to 4. In some embodiments, a linker polypeptide of the disclosure comprises or consists of the amino acid sequence (GGGGA)₂GGGGS. In some embodiments, a linker polypeptide comprises or consists of the amino acid sequence (GGGGQ)₂GGGGS. In some embodiments, a linker polypeptide comprises or consists of the amino acid sequence (GGGPS)₂GGGGS. In some embodiments, a linker polypeptide comprises or consists of the amino acid sequence GGGGS (PGGGS)₂. In some embodiments, a linker polypeptide comprises or consists of an amino acid sequence set forth in SEQ ID NOs: 7, 36, 38, 40, 42, 44, 46, 48, 50, or 52 in the Sequence Listing.

In addition, or alternatively, in some embodiments, the multivalent polypeptides and multivalent antibodies of the disclosure can include one or more RPTP-binding modules chemically linked to one or more receptor binding modules. In some embodiments, the multivalent polypeptides and multivalent antibodies of the disclosure can include (i) one or more RPTP-binding modules chemically linked to one or more receptor binding modules; and (ii) one or more RPTP-binding modules linked to one or more receptor binding modules via peptidyl linkages.

In some embodiments disclosed herein, at least one of the first and second polypeptide modules of the disclosed multivalent polypeptide or multivalent antibody includes an amino acid sequence for a protein-binding ligand or an antigen-binding moiety. In some embodiments, at least one of the first and second polypeptide modules includes an amino acid sequence for a protein-binding ligand. Generally, any suitable protein-binding ligands can be used for the compositions and methods of the present disclosure and can be, for example, any recombinant polypeptide or naturally-occurring polypeptide which has a specific binding affinity to a target antibody or a target protein (e.g., a recombinant or natural ligand of a receptor protein-tyrosine phosphatase (RPTP) or a cell surface receptor) (see, also, Verdoliva et al., J. Immuno. Methods, 2002; Naik et al., J. Chromatography, 2011). For example, non-limiting examples of suitable ligands for phosphatase CD45 include its natural ligands, such as e.g., lectin CD22 (Hermiston M L et al., Annu. Rev. Immunol. 2003) and Galactin-1 (Walzel H. et al., J. Immunol. Lett. 1999 and Nguyen J T et al. J Immunol. 2001). In some embodiments, at least one of the first and second polypeptide modules of the disclosed multivalent polypeptide or multivalent antibody include an amino acid sequence for one or more extracellular domains (ECDs) of a cell surface receptor or of a RPTP. Accordingly, in some embodiments, a first polypeptide module of the disclosed multivalent polypeptide includes one or more ECDs of a RPTP operably linked to a second module of the multivalent polypeptide. In some embodiments, a second polypeptide module of the disclosed multivalent polypeptide includes one or more ECDs of a cell surface receptor operably linked to a first module of the multivalent polypeptide.

As discussed above, non-limiting examples of protein-binding ligands suitable for the compositions and methods of the disclosure include natural ligands of a cell surface receptor. For example, suitable natural ligands for PD-1 include PD-L1 and PD-L2, which are members of the B7 family. Suitable natural ligands for CD5 include CD72, gp80-40 and Ig framework structures. As described in Example 18, a recombinant interleukin-2 (IL-2), which is a naturally-occurring ligand of IL-2R, can be operably linked to an anti-CD45 scFv to generate a multivalent polypeptide capable of binding to CD45 and IL-2R.

In addition or alternatively, the protein-binding ligand can be an agonist or an antagonist version of the target's natural ligand. Thus, in some embodiments, the protein-binding ligand is an agonist ligand of the receptor protein-tyrosine phosphatase (RPTP) or the cell surface receptor. In some other embodiments, the protein-binding ligand is an antagonist ligand of the receptor protein-tyrosine phosphatase (RPTP) or the cell surface receptor. In some embodiments, the protein-binding ligand can be a synthetic molecule such as, for example, peptides or small molecules.

In some embodiments, at least one of a first and a second polypeptide modules of the disclosed multivalent polypeptide or multivalent antibody includes an amino acid sequence for an antigen-binding moiety that binds to the target protein, e.g., a receptor protein-tyrosine phosphatase (RPTP) or a cell surface receptor. In some embodiments, the antigen-binding moiety includes one or more antigen-binding determinants of an antibody or a functional antigen-binding fragment thereof. Blocking antibodies and non-blocking antibodies are both suitable. As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-PD-1 antibody can prevent, inhibit, block, or reduce the binding interaction between PD-1 and PD-L1, thus preventing, blocking, inhibiting, or reducing the immunosuppressive functions associated with the PD-1/PD-L1 interaction. The term “non-blocking” antibody refers to an antibody that does not interfere, inhibits, blocks, or reduces biological or functional activity of the antigen to which it binds.

The term “antigen-binding fragment” as used herein refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (e.g., bivalent diabody -bi-scFv or divalent diabody -di-scFv), or a multispecific antibody formed from a portion of an antibody including one or more complementarity-determining regions (CDRs) of the antibody. The antigen-binding moiety can include naturally-derived polypeptides, antibodies produced by immunization of a non-human animal, or antigen-binding moieties obtained from other sources, e.g., camelids (see, e.g., Bannas et al. Front. Immunol., 22 Nov. 2017; McMahon C. et al., Nat Struct Mol Biol. 25(3): 289-296, 2018). The antigen-binding moiety can be engineered, synthesized, designed, humanized (see, e.g., Vincke et al., J. Biol. Chem. 30; 284(5): 3273-84, 2009), or modified so as to provide desired and/or improved properties.

Accordingly, in some embodiments, at least one of a first and a second polypeptide modules of the disclosed multivalent polypeptide or multivalent antibody includes an amino acid sequence for an antigen-binding moiety selected from the group consisting of antigen-binding fragments (Fab), single-chain variable fragments (scFv), nanobodies, V_{H} domains, V_{L} domains, single domain antibodies (dAb), V_{NAR} domains, and V_{H}H domains, diabodies, or a functional fragment of any one of the foregoing. In some embodiments, the antigen-binding moiety includes a single-chain variable fragment (scFv). In some embodiments, the antigen-binding moiety includes a diabody. In some embodiments, the antigen-binding moiety includes a bi-scFv or di-scFv, in which two scFv molecules are operably linked to each other. In some embodiments, the bi-scFv or di-scFv includes a single peptide chain with two V_{H} and two V_{L} regions, yielding tandem scFvs. In some embodiments, the antigen-binding moiety includes a nanobody. In some embodiments, the antigen-binding moiety includes a heavy chain variable region and a light chain variable region.

In some embodiments, the heavy chain variable region and the light chain variable region of the antigen-binding moiety are operably linked to each other via one or more intervening amino acid residues that are positioned between the heavy chain variable region and the light chain variable region. In some embodiments, the one or more intervening amino acid residues include a linker polypeptide sequence. In principle, there are no particular limitations to the length and/or amino acid composition of the linker polypeptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues. In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool or effect that would enhance or reduce the RPTP activity of the multivalent polypeptide. In some embodiments, the orientation and/or proximity of a first and a second polypeptide modules relative to one another can be optimize to create a partial antagonist to full antagonist versions of the multivalent polypeptide.

In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser) n, wherein n is an integer of one or more; and (Ser Gly Gly Gly Gly) n, wherein n is an integer of one or more. In some embodiments, the linker polypeptides are modified such that the amino acid sequence GSG (that occurs at the junction of traditional Gly/Ser linker polypeptide repeats) is not present. For example, in some embodiments, the polypeptide linker includes an amino acid sequence selected from the group consisting of: (GGGXX)nGGGGS and GGGGS(XGGGS)n, where X is any amino acid that can be inserted into the sequence and not result in a polypeptide including the sequence GSG, and n is 0 to 4. In some embodiments, the sequence of a linker polypeptide is (GGGX1X2)nGGGGS and X1 is P and X2 is S and n is 0 to 4. In some other embodiments, the sequence of a linker polypeptide is (GGGX1X2)nGGGGS and X1 is G and X2 is Q and n is 0 to 4. In some other embodiments, the sequence of a linker polypeptide is (GGGX1X2)nGGGGS and X1 is G and X2 is A and n is 0 to 4. In yet some other embodiments, the sequence of a linker polypeptide is GGGGS(XGGGS)n, and X is P and n is 0 to 4. In some embodiments, a linker polypeptide of the disclosure comprises or consists of the amino acid sequence (GGGGA)2GGGGS. In some embodiments, a linker polypeptide comprises or consists of the amino acid sequence (GGGGQ)2GGGGS. In some embodiments, a linker polypeptide comprises or consists of the amino acid sequence (GGGPS)2GGGGS. In some embodiments, a linker polypeptide comprises or consists of the amino acid sequence GGGGS (PGGGS)2. In yet a further embodiment, a linker polypeptide comprises or consists of an amino acid sequence set forth in SEQ ID NOs: 7, 36, 38, 40, 42, 44, 46, 48, 50, or 52 in the Sequence Listing.

In some embodiments, a first polypeptide module of the multivalent polypeptides and multivalent antibodies disclosed herein includes an antigen-binding moiety capable of binding one or more target RPTPs. Generally, there is no particular limitation on the RPTPs that can be targeted by the multivalent polypeptides and multivalent antibodies described herein. Non-limiting examples of suitable RPTPs include members of sub-families R1/R6, R2A, R2B, R3, R4. Members of sub-families R5, R7, and R8 are also suitable for the compositions and methods disclosed herein. Examples of suitable RPTPs include, but are not limited to, Ptpn5 (STEP), Ptpra (RPTP-α), Ptprb (PTPB), Ptprc (CD45), Ptprd (RPTP-δ), Ptpre (RPTP-R), Ptprf (LAR), Ptprg (RPTP-γ), Ptprh (SAP1), Ptprj (DEP-1), Ptprk (RPTP-κ), and functional variants of any thereof. Other non-limiting examples RPTPs suitable for the compositions and methods disclosed herein include Ptprm (RPTP-μ), Ptprn (IA2), Ptprn2 (IA2β), Ptpro (GLEPP1), Ptprp (PTPS31), Ptprr (PCPTP1), Ptprs (RPTP-σ), Ptprt (RPTP-ρ), Ptpru (RPTP-λ), Ptprz (RPTP-ζ), and functional variants of any thereof. In some embodiments, a first polypeptide module of the multivalent polypeptides and multivalent antibodies disclosed herein includes an antigen-binding moiety capable of binding CD45 phosphatase or a functional variant thereof, such as e.g., a homolog thereof. In some embodiments, the CD45 phosphatase is a human CD45 phosphatase. In general, any isoforms of CD45 can be used. In some embodiments, the receptor protein-tyrosine phosphatase is a CD45 isoform selected from the group consisting of CD45RA, CD45RB, CD45RC, CD45RAB, CD45RAC, CD45RBC, CD45R0, CD45R. Exemplary CD45-binding moieties suitable for the compositions and methods disclose herein include, but are not limited to those described in U.S. Pat. Nos. 7,825,222 and 9,701,756.

In some embodiments, the second polypeptide module of the multivalent polypeptides and multivalent antibodies disclosed herein includes an antigen-binding moiety capable of binding cell surface receptor that signals through a phosphorylation mechanism. Generally, the cell surface receptor can be any cell surface receptor known in the art. In some embodiments, the cell surface receptor is an immune-checkpoint receptor, a cytokine receptor, or a growth factor receptor. In some embodiments, the cell surface receptor is an immune-checkpoint receptor selected from the group consisting of inhibitory checkpoint receptors and stimulatory checkpoint receptors. In some embodiments, the cell surface receptor is an inhibitory checkpoint receptor. Generally, the inhibitory checkpoint receptor can be any one of inhibitory checkpoint receptors that signals through a phosphorylation mechanism. Non-limiting examples of inhibitory checkpoint receptors suitable for the compositions and methods disclosed herein include PD-1, CTLA-4, A2AR, B7-H3, B7-H4, BTLA, CD5, CD132, IDO, KIR, LAG3, TIM-3, TIGIT, VISTA, and functional variants thereof. In some embodiments, the inhibitory checkpoint receptor is PD-1 or a functional variant thereof. In some embodiments, the inhibitory checkpoint receptor is CTLA-4 or a functional variant thereof. In some embodiments, the inhibitory checkpoint receptor is TIGIT or a functional variant thereof. In some embodiments, the inhibitory checkpoint receptor is CD5 or a functional variant thereof. In some embodiments, the inhibitory checkpoint receptor is CD132 or a functional variant thereof.

In some embodiments, the cell surface receptor is a stimulatory checkpoint receptor. Generally, the stimulatory checkpoint receptor can be any one of stimulatory checkpoint receptors that signals through a phosphorylation mechanism. Non-limiting examples of stimulatory checkpoint receptors suitable for the compositions and methods disclosed herein include CD27, CD28, CD40, OX40, GITR, ICOS, CD137, and functional variants thereof. In some embodiments, the inhibitory checkpoint receptor is CD28 or a functional variant thereof.

In some embodiments, the cell surface receptors signals through a conserved amino acid motif that serves as a substrate for phosphorylation such as, for example, an immunoreceptor tyrosine-based activation motif (ITAM), or an immunoreceptor tyrosine-based switch motif (ITSM), or an immunoreceptor tyrosine-based inhibition motif (ITIM). In some embodiments, the cell surface receptor mediates signaling through a specific tyrosine-based motif selected from an ITAM motif, an ITSM motif, an ITIM motif, or a related intracellular motif that serves as a substrate for phosphorylation. In some embodiments, the cell surface receptor is selected from the group consisting of DAP10, DAP12, SIRPa, CD3, CD28, CD4, CD8, CD200, CD200R, ICOS, KIR, FCR, BCR, CD5, CD2, G6B, LIRs, CD7, and BTNs, or a functional variant of any thereof.

In some embodiments, the cell surface receptor is a cytokine receptor. In some embodiments, the cytokine receptor is selected from the group consisting of interleukin receptors, interferon receptors, chemokine receptors, growth hormone receptors, erythropoietin receptors (EpoRs), thymic stromal lymphopoietin receptors (TSLPRs), thrombopoetin receptors (TpoRs), granulocyte macrophage colony-stimulating factor (GM-CSF) receptors, granulocyte colony-stimulating factor (G-CSF) receptors.

In some embodiments, the cell surface receptor is a growth factor receptor. In some embodiments, the growth factor receptor is a tyrosine receptor kinase (TRK), which is also referred interchangeably herein as tyrosine kinase receptor (TKR). In general, the TRK can be any TRK known in the art. Non-limiting examples of TRKs suitable for the present disclosure include, but are not limited to, those belonging to RTK class I (EGF receptor family; ErbB family), RTK class II (Insulin receptor family), RTK class III (PDGF receptor family), RTK class IV (VEGF receptors family), RTK class V (FGF receptor family), RTK class VI (CCK receptor family), RTK class VII (NGF receptor family). Additional TRKs suitable for the invention disclosure include, but are not limited to, those belonging to RTK class VIII (HGF receptor family), RTK class IX (Eph receptor family), RTK class X (AXL receptor family), RTK class XI (TIE receptor family), RTK class XII (RYK receptor family), RTK class XIII (DDR receptor family), RTK class XIV (RET receptor family), RTK class XV (ROS receptor family), RTK class XVI (LTK receptor family), RTK class XVII (ROR receptor family), RTK class XVIII (MuSK receptor family), RTK class XIX (LMR receptor), RTK class XX. In some particular embodiments, the growth factor receptor is a stem cell growth factor receptor (SCFR) or an epidermal growth factor receptor (EGFR) selected from the group consisting of ErbB-1, ErbB-2 (HER2), ErbB-3, ErbB-4, and c-Kit (CD117).

Some embodiments disclosed herein relate to a multivalent polypeptide that includes (a) a domain A including a binding region of a heavy chain variable region of a first scFv specific for an epitope of the RPTP; (b) a domain B including a binding region of a light chain variable region of a second scFv specific for an epitope of the cell surface receptor; (c) a domain C including a binding region of a heavy chain variable region of the second scFv specific for an epitope of the cell surface receptor; and (d) a domain D including a binding region of a light chain variable region of the first scFv specific for an epitope of the RPTP.

Designation of the polypeptide domains of the disclosed multivalent polypeptides and multivalent antibodies as the “A”, “B”, “C”, or “D” polypeptide domains is not intended to imply any particular structural arrangement of the “first”, “second”, “third”, or “fourth” polypeptide domains within the disclosed multivalent polypeptides and multivalent antibodies. In addition or alternatively, the disclosed multivalent polypeptides and multivalent antibodies may include more than one polypeptide module capable of binding to a RPTP and/or a cell surface receptor. In some embodiments, the disclosed multivalent polypeptides and multivalent antibodies may include at least two polypeptide modules each capable of binding to a different RPTP. In some embodiments, the disclosed multivalent polypeptides and multivalent antibodies may include at least two polypeptide modules each capable of binding to the same RPTP. In some embodiments, the disclosed multivalent polypeptides and multivalent antibodies may include at least two polypeptide modules each capable of binding to a different cell surface receptor. In some embodiments, the disclosed multivalent polypeptides and multivalent antibodies may include at least two polypeptide modules each capable of binding to the same cell surface receptor.

In some embodiments, multiple receptor-binding modules are operably linked to a central RPTP-binding module to form a multivalent polypeptide or multivalent antibody having the general Formula (I).

(RPTP-binding module)−[linker−(receptor binding domain)]n Formula (I).

wherein n is an integer selected from the range of 1, 2, 3, 4, 5, 6, 7, 8, 9, or 10.

In some embodiments, multiple receptor-binding modules are operably linked in tandem to form a multivalent polypeptide or multivalent antibody having the general Formula (II).

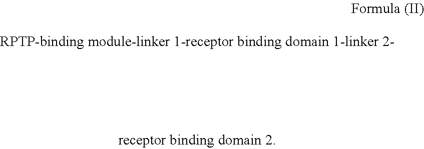

Some embodiments disclosed herein relate to a multivalent polypeptide that includes, in the N-terminal to C-terminal direction, (a) a domain A including a binding region of a heavy chain variable region of a first scFv specific for an epitope of the RPTP; (b) a domain B including a binding region of a light chain variable region of a second scFv specific for an epitope of the cell surface receptor; (c) a domain C including a binding region of a heavy chain variable region of the second scFv specific for an epitope of the cell surface receptor; and (d) a domain D including a binding region of a light chain variable region of the first scPv specific for an epitope of the RPTP.

A non-limiting list of exemplary polypeptides and antibodies described herein, such as multivalent polypeptides or multivalent antibodies of Formulae (1) and (11), are provided in Tables 1, 2, and 3.

TABLE 2
Exemplary bivalent polypeptides or bivalent, bispecific
antibodies of the present disclosure.

| Phosphatase | | | Binding |
|---|---|---|---|
| Binding | Linkage | Receptor Binding | modules |
| αCD45 | Peptidal | αPD-1 blocking-nivolumab based | scFv-scFv |
| αCD45 | Chemical | αPD-1 blocking-nivolumab based | scFv-scFv |
| αCD45 | Fc-Fusion | αPD-1 blocking-nivolumab based | scFv-scFv |
| αCD45 | Peptidal | αPD-1 blocking-Pembrolizumab based | scFv-scFv |
| αCD45 | Chemical | αPD-1 blocking-Pembrolizumab based | scFv-scFv |
| αCD45 | Fc-Fusion | αPD-1 blocking-Pembrolizumab based | scFv-scFv |
| αCD45 | Peptidal | αPD-1 blocking | scFv-scFv |
| αCD45 | Chemical | αPD-1 blocking | scFv-scFv |
| αCD45 | Fc-Fusion | αPD-1 blocking | scFv-scFv |
| αCD45 | Peptidal | αPD-1 non-blocking | scFv-scFv |
| αCD45 | Chemical | αPD-1 non-blocking | scFv-scFv |
| αCD45 | Fc-Fusion | αPD-1 non-blocking | scFv-scFv |
| αCD45 | Peptidal | αPD-1 blocking | scFv-VHH |
| αCD45 | Chemical | αPD-1 blocking | scFv-VHH |
| αCD45 | Fc-Fusion | αPD-1 blocking | scFv-VHH |
| αCD45 | Peptidal | αPD-1 non-blocking | scFv-VHH |
| αCD45 | Chemical | αPD-1 non-blocking | scFv-VHH |
| αCD45 | Fc-Fusion | αPD-1 non-blocking | scFv-VHH |
| αCD45 | Peptidal | αCD28 blocking | scFv-scFv |
| αCD45 | Chemical | αCD28 blocking | scFv-scFv |
| αCD45 | Fc-Fusion | αCD28 blocking | scFv-scFv |
| αCD45 | Peptidal | αCD28 non-blocking | scFv-scFv |
| αCD45 | Chemical | αCD28 non-blocking | scFv-scFv |
| αCD45 | Fc-Fusion | αCD28 non-blocking | scFv-scFv |
| αCD45 | Peptidal | αCD28 blocking | scFv-VHH |
| αCD45 | Chemical | αCD28 blocking | scFv-VHH |
| αCD45 | Fc-Fusion | αCD28 blocking | scFv-VHH |
| αCD45 | Peptidal | αCD28 non-blocking | scFv-VHH |
| αCD45 | Chemical | αCD28 non-blocking | scFv-VHH |
| αCD45 | Fc-Fusion | αCD28 non-blocking | scFv-VHH |
| αCD45 | Peptidal | αCTLA4 blocking | scFv-scFv |
| αCD45 | Chemical | αCTLA4 blocking | scFv-scFv |
| αCD45 | Fc-Fusion | αCTLA4 blocking | scFv-scFv |
| αCD45 | Peptidal | αCTLA4 non-blocking | scFv-scFv |
| αCD45 | Chemical | αCTLA4 non-blocking | scFv-scFv |
| αCD45 | Fc-Fusion | αCTLA4 non-blocking | scFv-scFv |
| αCD45 | Peptidal | αCTLA4 blocking | scFv-VHH |
| αCD45 | Chemical | αCTLA4 blocking | scFv-VHH |
| αCD45 | Fc-Fusion | αCTLA4 blocking | scFv-VHH |
| αCD45 | Peptidal | αCTLA4 non-blocking | scFv-VHH |
| αCD45 | Chemical | αCTLA4 non-blocking | scFv-VHH |
| αCD45 | Fc-Fusion | αCTLA4 non-blocking | scFv-VHH |
| αCD45 | Peptidal | αPD-1 blocking-nivolumab based | VHH-scFv |
| αCD45 | Chemical | αPD-1 blocking-nivolumab based | VHH-scFv |
| αCD45 | Fc-Fusion | αPD-1 blocking-nivolumab based | VHH-scFv |
| αCD45 | Peptidal | αPD-1 blocking-pembrolizumab based | VHH-scFv |
| αCD45 | Chemical | αPD-1 blocking-pembrolizumab based | VHH-scFv |
| αCD45 | Fc-Fusion | αPD-1 blocking-pembrolizumab based | VHH-scFv |
| αCD45 | Peptidal | αPD-1 blocking | VHH-scFv |
| αCD45 | Chemical | αPD-1 blocking | VHH-scFv |
| αCD45 | Fc-Fusion | αPD-1 blocking | VHH-scFv |
| αCD45 | Polypeptide | αPD-1 non-blocking | VHH-scFv |
| αCD45 | Chemical | αPD-1 non-blocking | VHH-scFv |
| αCD45 | Fc-Fusion | αPD-1 non-blocking | VHH-scFv |
| αCD45 | Peptidal | αPD-1 blocking | VHH-VHH |
| αCD45 | Chemical | αPD-1 blocking | VHH-VHH |
| αCD45 | Fc-Fusion | αPD-1 blocking | VHH-VHH |
| αCD45 | Peptidal | αPD-1 non-blocking | VHH-VHH |
| αCD45 | Chemical | αPD-1 non-blocking | VHH-VHH |
| αCD45 | Fc-Fusion | αPD-1 non-blocking | VHH-VHH |
| αCD45 | Peptidal | αCD28 blocking | VHH-scFv |
| αCD45 | Chemical | αCD28 blocking | VHH-scFv |
| αCD45 | Fc-Fusion | αCD28 blocking | VHH-scFv |
| αCD45 | Peptidal | αCD28 non-blocking | VHH-scFv |
| αCD45 | Chemical | αCD28 non-blocking | VHH-scFv |
| αCD45 | Fc-Fusion | αCD28 non-blocking | VHH-scFv |
| αCD45 | Peptidal | αCD28 blocking | VHH-VHH |
| αCD45 | Chemical | αCD28 blocking | VHH-VHH |
| αCD45 | Fc-Fusion | αCD28 blocking | VHH-VHH |
| αCD45 | Peptidal | αCD28 non-blocking | VHH-VHH |
| αCD45 | Chemical | αCD28 non-blocking | VHH-VHH |
| αCD45 | Fc-Fusion | αCD28 non-blocking | VHH-VHH |
| αCD45 | Peptidal | αCTLA4 blocking | VHH-scFv |
| αCD45 | Chemical | αCTLA4 blocking | VHH-scFv |
| αCD45 | Fc-Fusion | αCTLA4 blocking | VHH-scFv |
| αCD45 | Peptidal | αCTLA4 non-blocking | VHH-scFv |
| αCD45 | Chemical | αCTLA4 non-blocking | VHH-scFv |
| αCD45 | Fc-Fusion | αCTLA4 non-blocking | VHH-scFv |
| αCD45 | Peptidal | αCTLA4 blocking | VHH-VHH |
| αCD45 | Chemical | αCTLA4 blocking | VHH-VHH |
| αCD45 | Fc-Fusion | αCTLA4 blocking | VHH-VHH |
| αCD45 | Peptidal | αCTLA4 non-blocking | VHH-VHH |
| αCD45 | Chemical | αCTLA4 non-blocking | VHH-VHH |
| αCD45 | Fc-Fusion | αCTLA4 non-blocking | VHH-VHH |

TABLE 3
Exemplary trivalent polypeptides or trivalent antibodies
of the present disclosure (either scFv- or VHH-based).

| Phosphatase | | | |
|---|---|---|---|
| Binding | Linkage | Receptor Binding #1 | Receptor Binding #2 |
| αCD45 | Peptidal | αPD-1 blocking | αCTLA4 blocking |
| αCD45 | Chemical | αPD-1 blocking | αCTLA4 blocking |
| αCD45 | Fc-Fusion | αPD-1 blocking | αCTLA4 blocking |
| αCD45 | Peptidal | αPD-1 non-blocking | αCTLA4 blocking |
| αCD45 | Chemical | αPD-1 non-blocking | αCTLA4 blocking |
| αCD45 | Fc-Fusion | αPD-1 non-blocking | αCTLA4 blocking |
| αCD45 | Peptidal | αPD-1 blocking | αCTLA4 non-blocking |
| αCD45 | Chemical | αPD-1 blocking | αCTLA4 non-blocking |
| αCD45 | Fc-Fusion | αPD-1 blocking | αCTLA4 non-blocking |
| αCD45 | Peptidal | αPD-1 non-blocking | αCTLA4 non-blocking |
| αCD45 | Chemical | αPD-1 non-blocking | αCTLA4 non-blocking |
| αCD45 | Fc-Fusion | αPD-1 non-blocking | αCTLA4 non-blocking |

In some embodiments disclosed herein, the multivalent polypeptide includes an amino acid sequence that has at least 80% sequence identity to an amino acid sequence selected from the group consisting of SEQ ID NOS: 2, 4, 6, 10, 12, 14, 16, 20, 22, 24, 26, 28, and 54, or a functional fragment thereof. In some embodiments, the multivalent polypeptide includes an amino acid sequence that has at least 85%, at least 90%, at least 95%, at least 96%, at least 97%, at least 98%, at least 99%, or 100% sequence identity to an amino acid sequence selected from the group consisting of SEQ ID NOS: 2, 4, 6, 10, 12, 14, 16, 20, 22, 24, 26, 28, and 54, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 2, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 4, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 6, or a functional fragment thereof.

In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 10, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 12, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 14, or a functional fragment thereof.

In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 16, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 18, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 20, or a functional fragment thereof.

In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 22, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 24, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 26, or a functional fragment thereof.

In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 28, or a functional fragment thereof. In some embodiments, the multivalent polypeptide includes an amino acid sequence that has at least 80%, at least 85%, at least 90%, at least 95%, at least 96%, at least 97%, at least 98%, at least 99%, or 100% sequence identity to the amino acid sequence of SEQ ID NO: 54, or a functional fragment thereof.

In some particular embodiments, the multivalent polypeptide of the present disclosure can be a multivalent antibody (e.g., bivalent antibody or trivalent antibody) including at least two antigen-binding moieties each possessing specific binding for a target protein. In some embodiments, the at least two antigen-binding moieties possess specific binding for the same target protein. Such antibody is multivalent, monospecific antibody. In some embodiments, the at least two antigen-binding moieties possessing specific binding for at least two different target proteins. Such antibody is multivalent, multispecific antibody (e.g., bispecific, trispecific, etc.) Accordingly, some embodiments disclosed herein relate to a multivalent antibody or functional fragment thereof, which includes (i) a first polypeptide module specific for one or more receptor protein-tyrosine phosphatase (RPTP), and (ii) a second polypeptide module specific for one or more cell surface receptor that signals through a phosphorylation mechanism, wherein the first polypeptide module is operably linked to the second polypeptide module. Accordingly, in some embodiments, the disclosed multivalent antibody can be a bivalent, monospecific antibody. In some embodiments, the disclosed multivalent antibody can be a trivalent, monospecific antibody. In some embodiments, the disclosed multivalent antibody can be a bivalent, bispecific antibody. In some embodiments, the disclosed multivalent antibody can be a trivalent, trispecific antibody.

One skilled in the art will appreciate that the complete amino acid sequence can be used to construct a back-translated gene. For example, a DNA oligomer containing a nucleotide sequence coding for a given polypeptide can be synthesized. For example, several small oligonucleotides coding for portions of the desired polypeptide can be synthesized and then ligated. The individual oligonucleotides typically contain 5′ or 3′ overhangs for complementary assembly.

In addition to generating mutant polypeptides via expression of nucleic acid molecules that have been altered by recombinant molecular biological techniques, a subject multivalent polypeptide or multivalent antibody in accordance with the present disclosure can be chemically synthesized. Chemically synthesized polypeptides are routinely generated by those of skill in the art.

Once assembled (by synthesis, site-directed mutagenesis or another method), the DNA sequences encoding a multivalent polypeptide or multivalent antibody as disclosed herein will be inserted into an expression vector and operably linked to an expression control sequence appropriate for expression of the multivalent polypeptide or multivalent antibody in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host.

The binding activity of the multivalent polypeptides and multivalent antibodies of the disclosure can be assayed by any suitable method known in the art. For example, the binding activity of the multivalent polypeptides and multivalent antibodies of the disclosure can be determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays. An antibody or polypeptide that “preferentially binds” or “specifically binds” (used interchangeably herein) to a target protein or target epitope is a term well understood in the art, and methods to determine such specific or preferential binding are also known in the art. An antibody or polypeptide is said to exhibit “specific binding” or “preferential binding” if it reacts or associates more frequently, more rapidly, with greater duration and/or with greater affinity with a particular protein or epitope than it does with alternative proteins or epitopes. An antibody or polypeptide “specifically binds” or “preferentially binds” to a target if it binds with greater affinity, avidity, more readily, and/or with greater duration than it binds to other substances. Also, an antibody or polypeptide “specifically binds” or “preferentially binds” to a target if it binds with greater affinity, avidity, more readily, and/or with greater duration to that target in a sample than it binds to other substances present in the sample. For example, an antibody or polypeptide that specifically or preferentially binds to a PD-1 epitope is an antibody or polypeptide that binds this epitope with greater affinity, avidity, more readily, and/or with greater duration than it binds to other PD-1 epitopes or non-PD-1 epitopes. It is also understood by reading this definition, for example, that an antibody or polypeptide (or moiety or epitope) which specifically or preferentially binds to a first target may or may not specifically or preferentially bind to a second target. As such, “specific binding” or “preferential binding” does not necessarily require (although it can include) exclusive binding.

A variety of assay formats may be used to select an antibody or polypeptide that specifically binds a molecule of interest. For example, solid-phase ELISA immunoassay, immunoprecipitation, Biacore™ (GE Healthcare, Piscataway, NJ), KinExA, fluorescence-activated cell sorting (FACS), Octet™ (ForteBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Generally, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is <7 nM.

The term “binding affinity” is herein used as a measure of the strength of a non-covalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant kₐ (or k_{off}), respectively. K_{D} is related to kₐ and kₐ through the equation K_{D}=kₐ/kₐ. The value of the dissociation constant can be determined directly by well-known methods, and can be computed even for complex mixtures by methods such as those set forth in Caceci et al. (1984, Byte 9:340-362). For example, the K_{D} may be established using a double-filter nitrocellulose filter binding assay such as that disclosed by Wong & Lohman (1993, Proc. Natl. Acad. Sci. USA 90:5428-5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g., by using a Biacore™ system, or KinExA.

### Nucleic Acid Molecules

In one aspect, some embodiments disclosed herein relate to recombinant nucleic acid molecules encoding the multivalent polypeptides and multivalent antibodies of the disclosure, expression cassettes, and expression vectors containing these nucleic acid molecules operably linked to regulator sequences which allow expression of the multivalent polypeptides and multivalent antibodies in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

In some embodiments disclosed herein, the nucleic acid molecules of the disclosure include a nucleotide sequence encoding a multivalent polypeptide which include (i) a first amino acid sequence including a first polypeptide module capable of binding to a receptor protein-tyrosine phosphatase (RPTP), and (ii) a second amino acid sequence including a second polypeptide module capable of binding to a cell surface receptor that signals through a phosphorylation mechanism, wherein the first polypeptide module is operably linked to the second polypeptide module. In some embodiments, the nucleic acid molecules of the disclosure include a nucleotide sequence encoding a multivalent antibody which includes a (i) a first polypeptide module specific for one or more receptor protein-tyrosine phosphatases (RPTP), and (ii) a second polypeptide module specific for one or more cell surface receptors that signal through a phosphorylation mechanism.

In some embodiments disclosed herein, the nucleic acid molecules include a nucleotide sequence encoding a polypeptide that includes (i) an amino acid sequence having at least 80% sequence identity to the amino acid sequence of a multivalent polypeptide as disclosed herein or a functional fragment thereof; or (ii) an amino acid sequence having at least 80% sequence identity to the multivalent antibody of or a functional fragment thereof as disclosed herein. The nucleic acid molecules include a nucleotide sequence encoding a polypeptide that includes (i) an amino acid sequence having at least 90%, at least 95%, at least 96%, at least 97%, at least 98%, at least 99%, or 100% sequence identity to the amino acid sequence of a multivalent polypeptide as disclosed herein or a functional fragment thereof; or (ii) an amino acid sequence having at least 90%, at least 95%, at least 96%, at least 97%, at least 98%, at least 99%, or 100% sequence identity to the multivalent antibody of or a functional fragment thereof as disclosed herein.

In some embodiments, the nucleic acid molecules include a nucleotide sequence that has at least 80%, at least 85%, at least 90%, at least 95%, at least 96%, at least 97%, at least 98%, at least 99%, or 100% sequence identity to a nucleotide sequence selected from the group consisting of SEQ ID NOS: 1, 3, 5, 9, 11, 13, 15, 19, 21, 23, 25, 27, and 53 or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 1, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 3, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 5, or a functional fragment thereof.

In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 9, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 11, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 13, or a functional fragment thereof.

In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 15, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 19, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 21, or a functional fragment thereof.

In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 23, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 25, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 27, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 53, or a functional fragment thereof.

Some embodiments disclosed herein relate to vectors or expression cassettes including a recombinant nucleic acid molecule as disclosed herein. As used herein, the term “expression cassette” refers to a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. As such, the term expression cassette may be used interchangeably with the term “expression construct”.

Also provided herein are vectors, plasmids or viruses containing one or more of the nucleic acid molecules encoding any of the multivalent polypeptides and multivalent antibodies disclosed herein. The nucleic acid molecules described above can be contained within a vector that is capable of directing their expression in, for example, a cell that has been transduced with the vector. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the multivalent polypeptides and multivalent antibodies of the present disclosure to be amplified in copy number. Such amplifiable vectors are known in the art. They include, for example, vectors able to be amplified by DHFR amplification (see, e.g., Kaufman, U.S. Pat. No. 4,470,461) or glutamine synthetase (“GS”) amplification (see, e.g., U.S. Pat. No. 5,122,464 and European published application EP 338,841).

Accordingly, in some embodiments, the multivalent polypeptides and multivalent antibodies of the present disclosure can be expressed from vectors, generally expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

Exemplary recombinant expression vectors can include one or more regulatory sequences, selected on the basis of the host cells to be used for expression, operably linked to the nucleic acid sequence to be expressed.

DNA vector can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

The nucleic acid sequences encoding the multivalent polypeptides and multivalent antibodies of the present disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the multivalent polypeptides and multivalent antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells. In some embodiments nucleic acid inserts, which encode the subject multivalent polypeptide or multivalent antibody in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject multivalent polypeptide or multivalent antibody, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the case of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma virus, adenovirus and cytomegalovirus. Non-limiting examples of useful expression vectors for bacterial hosts include known bacterial plasmids, such as plasmids from E. coli, including col E1, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2μ plasmid and derivatives thereof. Non-limiting examples of useful vectors for insect cells include pVL 941 and pFastBac™ 1.

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans will readily appreciate numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject multivalent polypeptide or multivalent antibody disclosed herein are also features of the disclosure. A cell of the disclosure is a transfected cell, e.g., a cell into which a nucleic acid molecule, for example a nucleic acid molecule encoding a mutant IL-2 polypeptide, has been introduced by means of recombinant DNA techniques. The progeny of such a cell are also considered within the scope of the disclosure.

The precise components of the expression system are not critical. For example, an multivalent polypeptide or multivalent antibody as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, multivalent polypeptides or multivalent antibodies obtained will be glycosylated or unglycosylated depending on the host organism used to produce the multivalent polypeptides or multivalent antibodies. If bacteria are chosen as the host then the multivalent polypeptide or multivalent antibody produced will be unglycosylated. Eukaryotic cells, on the other hand, will glycosylate the multivalent polypeptides or multivalent antibodies, although perhaps not in the same way as native polypeptides is glycosylated. The multivalent polypeptides or multivalent antibodies produced by the transformed host can be purified according to any suitable methods known in the art. Produced multivalent polypeptides or multivalent antibodies can be isolated from inclusion bodies generated in bacteria such as E. coli, or from conditioned medium from either mammalian or yeast cultures producing a given multivalent polypeptide or multivalent antibody using cation exchange, gel filtration, and or reverse phase liquid chromatography.

In addition or alternatively, another exemplary method of constructing a DNA sequence encoding the multivalent polypeptides or multivalent antibodies of the disclosure is by chemical synthesis. This includes direct synthesis of a peptide by chemical means of the protein sequence encoding for a multivalent polypeptide or multivalent antibody exhibiting the properties described. This method can incorporate both natural and unnatural amino acids at positions that affect the binding affinity of the multivalent polypeptide or multivalent antibody with the target protein. Alternatively, a gene which encodes the desired multivalent polypeptide or multivalent antibody can be synthesized by chemical means using an oligonucleotide synthesizer. Such oligonucleotides are designed based on the amino acid sequence of the desired multivalent polypeptide or multivalent antibody, and generally selecting those codons that are favored in the host cell in which the recombinant multivalent polypeptide or multivalent antibody will be produced. In this regard, it is well recognized in the art that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular multivalent polypeptide or multivalent antibody, there will be many DNA degenerate sequences that will code for that multivalent polypeptide or multivalent antibody. For example, it will be appreciated that in addition to the DNA sequences for multivalent polypeptides or multivalent antibodies provided in the Sequence Listing, there will be many degenerate DNA sequences that code for the multivalent polypeptides or multivalent antibodies disclosed herein. These degenerate DNA sequences are considered within the scope of this disclosure. Therefore, “degenerate variants thereof” in the context of this disclosure means all DNA sequences that code for and thereby enable expression of a particular multivalent polypeptide or multivalent antibody.

The DNA sequence encoding the subject multivalent polypeptide or multivalent antibody, whether prepared by site directed mutagenesis, chemical synthesis or other methods, can also include DNA sequences that encode a signal sequence. Such signal sequence, if present, should be one recognized by the cell chosen for expression of the multivalent polypeptide or multivalent antibody. It can be prokaryotic, eukaryotic or a combination of the two. In general, the inclusion of a signal sequence depends on whether it is desired to secrete the multivalent polypeptide or multivalent antibody as disclosed herein from the recombinant cells in which it is made. If the chosen cells are prokaryotic, the DNA sequence generally does not encode a signal sequence. If the chosen cells are eukaryotic, a signal sequence is generally included.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a mutant IL-2) is incorporated into a vector (e.g., a plasmid or viral vector) or into the genome of a heterologous cell (or the genome of a homologous cell, at a position other than the natural chromosomal location).

### Pharmaceutical Compositions

In some embodiments, the multivalent polypeptides and multivalent antibodies of the present disclosure can be incorporated into compositions, including pharmaceutical compositions. Such compositions typically include the multivalent polypeptides and/or multivalent antibodies and a pharmaceutically acceptable excipient.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the common methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., multivalent polypeptides, multivalent antibodies, and/or nucleic acid molecules of the disclosure) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject multivalent polypeptides and multivalent antibodies of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject multivalent polypeptides and multivalent antibodies of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the multivalent polypeptides and multivalent antibodies of the disclosure can also be prepared in the form of suppositories (e.g., with conventional suppository bases such as cocoa butter and other glycerides) or retention enemas for rectal delivery.

In some embodiments, the multivalent polypeptides and multivalent antibodies of the disclosure can also be administered by transfection or infection using methods known in the art, including but not limited to the methods described in McCaffrey et al. (Nature 418:6893, 2002), Xia et al. (Nature Biotechnol. 20:1006-1010, 2002), or Putnam (Am. J. Health Syst. Pharm. 53:151-160, 1996, erratum at Am. J. Health Syst. Pharm. 53:325, 1996).

In some embodiments, the subject multivalent polypeptides and multivalent antibodies of the disclosure are prepared with carriers that will protect the multivalent polypeptides and multivalent antibodies against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

As described in greater detail below, the multivalent polypeptides and multivalent antibodies of the present disclosure may also be modified to achieve extended duration of action such as by PEGylation, acylation, Fc fusions, linkage to molecules such as albumin, etc. In some embodiments, the multivalent polypeptides or multivalent antibodies can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (“PEG”) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the multivalent polypeptides and multivalent antibodies of the disclosure.

Accordingly, in some embodiments, the multivalent polypeptides and multivalent antibodies of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the multivalent polypeptide or multivalent antibody. In some embodiments, the PEGylated or PASylated multivalent polypeptide or multivalent antibody contains a PEG or PAS moiety on only one amino acid. In other embodiments, the PEGylated or PASylated multivalent polypeptide or multivalent antibody contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated multivalent polypeptide or multivalent antibody may be coupled directly to PEG or PAS (e.g., without a linking group) through an amino group, a sulfhydryl group, a hydroxyl group, or a carboxyl group. In some embodiments, the multivalent polypeptide or multivalent antibody of the disclosure is covalently bound to a polyethylene glycol with an average molecular weight of 20,000 Daltons.

In some embodiments, the multivalent polypeptides or multivalent antibodies of the disclosure can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (“PEG”) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

### Methods of Treatment

Administration of any one of the therapeutic compositions described herein, e.g., multivalent polypeptides, multivalent antibodies, nucleic acids, recombinant cells, cell cultures, and pharmaceutical compositions, can be used in the treatment of relevant diseases, such as cancers and chronic infections. In some embodiments, the multivalent polypeptides, multivalent antibodies, nucleic acids, recombinant cells, cell cultures, and/or pharmaceutical compositions as described herein can be incorporated into therapeutic agents for use in methods of treating an individual who has, who is suspected of having, or who may be at high risk for developing one or more health diseases or autoimmune diseases associated with checkpoint inhibition. Exemplary autoimmune diseases and health diseases can include, without limitation, cancers and chronic infection.

Accordingly, in one aspect, some embodiments of the disclosure relate to methods for modulating cell signaling mediated by a cell surface receptor that signals through a phosphorylation mechanism in a subject, the method includes administering to the subject a first therapy including an effective amount of (i) a multivalent polypeptide as disclosed herein, or (ii) a multivalent antibody as disclosed herein. In another aspect, some embodiments of the disclosure relate to methods for the treatment of a health disease in a subject in need thereof, the method including administering to the subject a first therapy including an effective amount of (i) a multivalent polypeptide as disclosed herein, or (ii) a multivalent antibody as disclosed herein.

A pharmaceutical composition is formulated to be compatible with its intended route of administration. The multivalent polypeptides and multivalent antibodies of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject multivalent polypeptides and multivalent antibodies of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies generally within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC50 (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions described herein, e.g., multivalent polypeptides, multivalent antibodies, nucleic acids, recombinant cells, cell cultures, and pharmaceutical compositions, can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to multivalent polypeptides or multivalent antibodies, the therapeutically effective amount of a multivalent polypeptide or multivalent antibody of the disclosure (e.g., an effective dosage) depends on the multivalent polypeptide or multivalent antibody selected. For instance, single dose amounts in the range of approximately 0.001 to 0.1 mg/kg of patient body weight can be administered; in some embodiments, about 0.005, 0.01, 0.05 mg/kg may be administered.

In one aspect, provided herein is a method for modulating cell signaling mediated by a cell surface receptor that signals through a phosphorylation mechanism in a subject. The method is performed by administering to the subject an effective amount of (i) a multivalent polypeptide as disclosed herein, or (ii) a multivalent antibody as disclosed herein. In another aspect, provided herein is a method for the treatment of a disease in a subject in need thereof. The method is performed by administering to the subject an effective amount of (i) a multivalent polypeptide as disclosed herein, or (ii) a multivalent antibody as disclosed herein.

As discussed supra, a therapeutically effective amount includes an amount of a therapeutic composition that is sufficient to promote a particular effect when administered to a subject, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the administered multivalent polypeptide or the multivalent antibody recruits an RPTP activity into spatial proximity of a cell surface receptor, eliciting phosphatase activity that reduces the phosphorylation level of the cell surface receptor. In some embodiments, the administered multivalent polypeptide or the multivalent antibody recruits the RPTP into spatial proximity of a cell surface receptor, e.g., the distance between the RPTP and the cell surface receptor is less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the administered multivalent polypeptide or the multivalent antibody recruits the RPTP into spatial proximity such that the RPTP is about 10 to 100 angstroms from the cell surface receptor. In some embodiments, the spatial proximity amounts to less than about 100 angstroms. In some embodiments, the distance between the RPTP and the cell surface receptor is less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

In some embodiments, when the RPTP and cell surface receptor are brought into a spatial proximity of one to another, the phosphorylation level of the cell surface receptor can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the phosphorylation level of the cell surface receptor in an untreated subject under similar conditions.

In some embodiments, the administration of the multivalent polypeptide or the multivalent antibody confers a reduced activity of an immune checkpoint receptor in the subject. The reduction in activity of the immune checkpoint receptor can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the activity of the immune checkpoint receptor in an untreated subject under similar conditions.

In some embodiments of the disclosed methods, the administration of the multivalent polypeptide or the multivalent antibody confers an enhancement in T-cell activity in the subject. The T-cell activity can be enhanced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the T-cell activity in an untreated subject under similar conditions. In some embodiments, the enhancement in T-cell activity is determined by increase in up-regulation of CD69 and/or CD25 in activated T cells. In some embodiments, the enhancement in T-cell activity is determined by increase in IL-2 secretion in activated T cells. In some embodiments, the enhancement in T-cell activity is determined by increase in production in activated T cells.

In some embodiments of the disclosed methods, the subject is a mammal. In some embodiments, the mammal is human. In some embodiments, the subject has or is suspected of having a disease associated with inhibition of cell signaling mediated by a cell surface receptor. The diseases suitable for being treated by the compositions and methods of the disclosure include, but are not limited to, cancers, autoimmune diseases, inflammatory diseases, and infectious diseases. In some embodiments, the disease is a cancer or a chronic infection.

As discussed supra, any one of the multivalent polypeptides, multivalent antibodies, nucleic acids, recombinant cells, cell cultures, and/or pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antincoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antincoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes. Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

In some aspects, the one or more anti-cancer therapies include radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of precancers or normal tissues is also contemplated herein.

Accordingly, in some embodiments, the disclosed treatment methods further include administering to the subject a second therapy. Generally, the second therapy can be any therapy known in the art. Non-limiting examples of therapies suitable for use in combination with the therapeutic compositions disclosed herein include chemotherapy, radiotherapy, immunotherapy, hormonal therapy, toxin therapy, and surgery. In some embodiments, the second therapy includes one or more additional therapeutic agents such as, for example, chemotherapeutics or anti-cancer agents or anti-cancer therapies as described above. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapeutic agent and the second therapy are administered together in a single formulation.

### Systems or Kits

Systems or kits of the present disclosure include one or more of any of the polypeptides, antibodies, nucleic acids, vectors, or pharmaceutical compositions disclosed herein as well as syringes (including pre-filled syringes) and/or catheters (including pre-filled syringes) used to administer any of the multivalent polypeptides, multivalent antibodies, nucleic acids, vectors, or pharmaceutical composition to an individual. The kits also include written instructions for using of any of the multivalent polypeptides, multivalent antibodies, nucleic acids, vectors, or pharmaceutical composition disclosed herein as well as syringes and/or catheters for use with their administration.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

This Example describes experiments performed to demonstrate that cell surface receptor signaling can be modulated by local phosphatase recruitment in accordance with some embodiments of the disclosure.

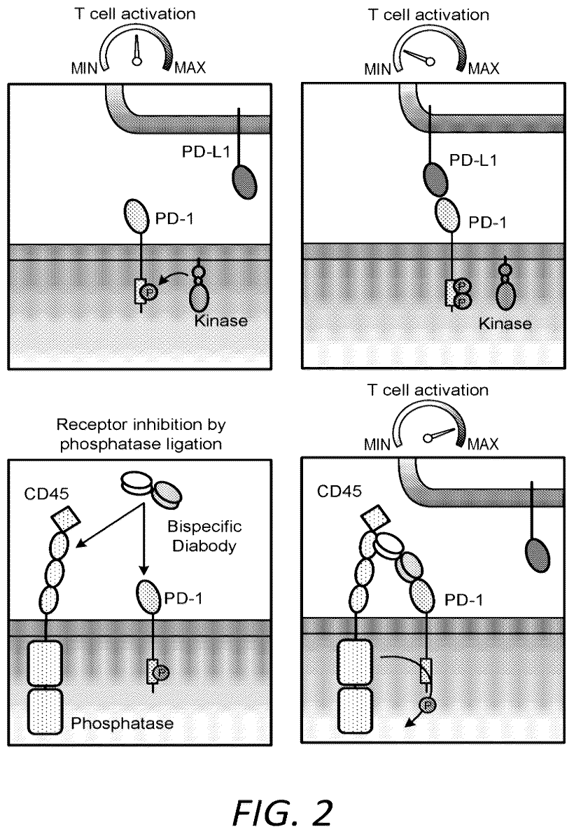

As shown in FIG. 2, cell surface receptors, such as PD-1 and checkpoint receptors, at the cell membrane undergo low, basal, levels of phosphorylation in the resting, unliganded state (top left panel of FIG. 2). Binding to cognate ligands increases phosphorylation and augments signaling to inhibit T-cell activation (top right panel of FIG. 2). As an example, PD-1 blocking antibodies, “checkpoint inhibitors” impair receptor/ligand interaction to increase T-cell activation, but basal receptor signaling is unaffected, thus the enhancement of T-cell activation by checkpoint Ab blockade is limited in its effectiveness. In the current invention, a bispecific diabody that recruits CD45 phosphatase to the spatial proximity of receptors of interest is expected to reduce phosphorylation of PD-1 in both resting and ligand activated states (bottom panels of FIG. 2).

### Construction of a Bispecific Diabody Targeting Human CD45 and PD-1

A bispecific diabody targeting human CD45 and PD-1 was constructed, where the amino acid sequence of the antibody includes, in N-terminal to C-terminal direction: (i) a heavy chain variable region of a scFv specific for CD45, (ii) a light chain variable region of a scFv specific for cell surface receptor PD-1; (iii) a heavy chain variable region of the scFv specific for cell surface receptor PD-1; and a light chain variable region of the scFv specific for CD45. The amino acid sequence of the antibody is disclosed in SEQ ID NO: 2 of the Sequence Listing.

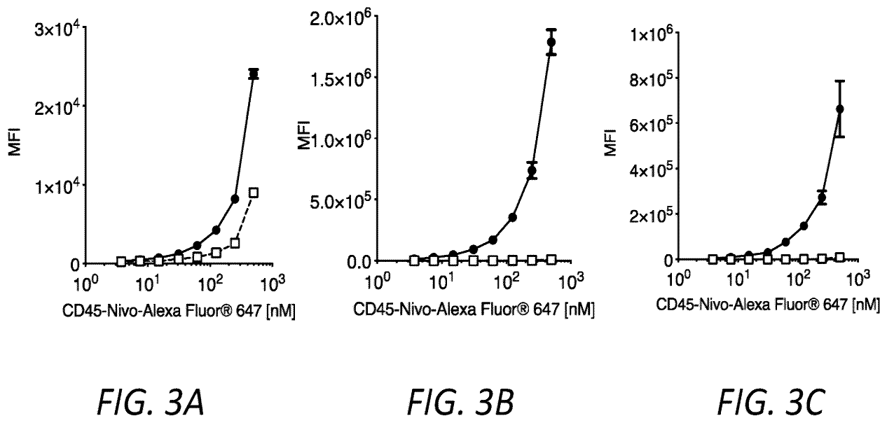

As shown in FIGS. 3A-3C, the bispecific diabody CD45/PD-1 constructed as described above was demonstrated to bind to HEK293 cells transfected with CD45 (FIG. 3A), PD-1 (FIG. 3B) or both molecules (FIG. 3C). In these experiments, approximately one million HEK293 cells were transfected with 1 μg of CD45 RA, PD-1 or both. Approximately 24 hours after transfection, cells were harvested and stained at the indicated concentrations for 45 minutes on ice with CD45/PD-1 bispecific diabody previously labelled with Alexa Fluor® 647 fluorescent dye as per manufacturer protocol (Thermo Fisher Scientific, Sunnyvale, USA). Untransfected HEK293 cells were stained at the indicated concentrations (grey line). Quantification of surface staining was performed by FACS (CytoFLEX, Beckman Coulter, Indianapolis, USA). Representative data is shown as mean±SD, n=3.

Another set of experiments was performed to demonstrate that the bispecific diabody CD45/PD-1 described above was capable of binding to the extracellular region of human CD45 and human PD-1. In these experiments, an anti-human RIPR-PD1 multivalent antibody, αCD45-PD1 (Nivo) was designed and constructs. In this antibody, the bispecific module was composed of an anti-CD45 scFv operably linked to an anti-PD1 scFv corresponding to nivolumab sequence. This bispecific diabody CD45/PD-1 was purified by size-exclusion (AKTA FPLC, GE Healthcare, Superdex 200 Increase; 280 nm absorbance). In addition, protein integrity and purity of the bispecific diabody CD45/PD-1 were confirmed by non-reducing SDS-PAGE electrophoresis followed by standard Coomassie staining (data not shown). Using surface plasmon resonance (SPR) technique, αCD45-PD1 (Nivo) was determined to bind to the extracellular region of human CD45 and human PD-1, where the affinity (K_{D}) for CD45 was found to be approximately 300 nM and the affinity for PD-1 was found to be approximately 6 nM.

Another exemplary multivalent antibody capable of binding to phosphatase CD45 and cell surface receptor PD-1 in accordance with some embodiments of the disclosure was constructed. The amino acid sequence of this multivalent polypeptide includes, in the N-terminal to C-terminal direction: (i) a heavy chain variable region of a scFv specific for CD45, (ii) a light chain variable region of the scFv specific for PD-1; (iii) a heavy chain variable region of the scFv specific for PD-1; and (iv) a light chain variable region of the scFv specific for CD45. The amino acid sequence of the multivalent antibody is disclosed in SEQ ID NO: 12 of the Sequence Listing.

### Example 2

This Example describes experiments performed to demonstrate that PD-1 expression reduces T-cell activation even in the absence of PD-1 ligands.

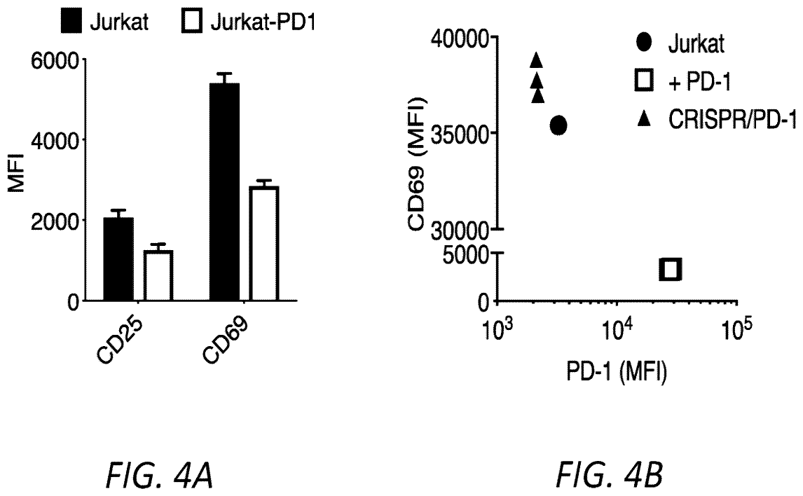

In these experiments, Jurkat T cells were lentivirally transduced with full-length wild-type PD-1 and surface expression of PD-1 was determined by FACS performed with an anti-PD1 antibody (clone EH12.2H7, Biolegend). Approximately 56% of the Jurkat T cells transduced with full-length wild-type PD-1 were found to display PD-1 at the cell surface. Jurkat T cells expressing PD-1 (Jurkat-PD1) at approx. 1 million/ml cell density were activated with immobilized Muromonab-CD3 (Orthoclone OKT3) at 2 μg/ml overnight in 96-well plates. As shown in FIG. 4A, the up-regulation of CD25 and CD69 (Biolegend) triggered by the overnight incubation with OKT3 was found to be lower for cells expressing PD-1. As shown in FIG. 4B, it was observed that reduced PD-1 expression in cells treated with CRISPR/Cas9 PD-1 targeted guide RNA leads to higher CD69 expression upon activation with OKT3.

In these experiments, a DNA sequence targeting the human PD-1 sequence (5′-CACCGCGACTGGCCAGGGCGCCTGT-3′; SEQ ID NO: 8) was cloned in the CRISPR/Cas9 lentiviral delivery backbone (Addgene, Plasmid #52961). Jurkat T cells were transduced with PD-1/CRISPR/Cas9 seven days prior to the OKT3 stimulation assay. Representative data is shown as mean±SEM, n=3.

### Example 3

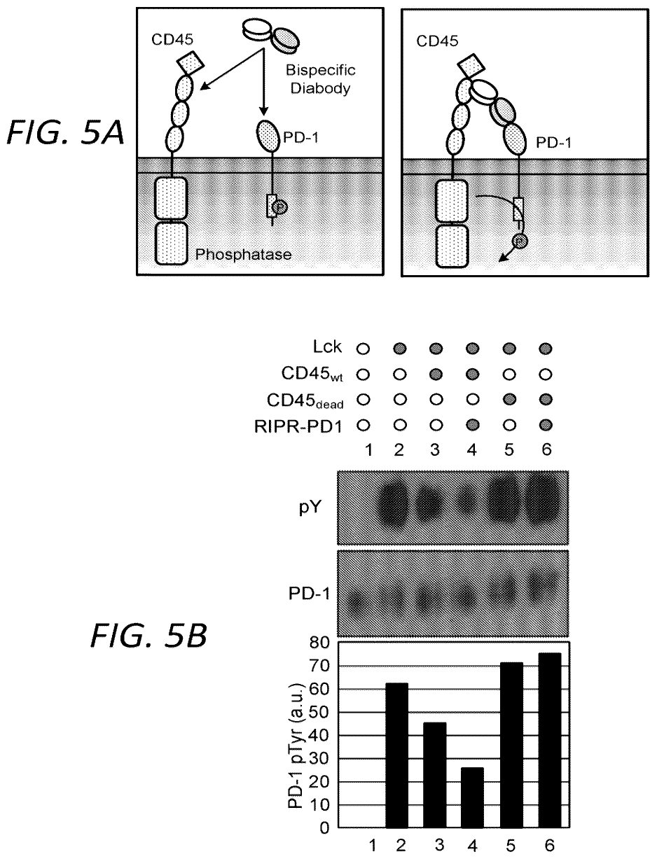

This Example describes experiments performed to illustrate a reconstitution of PD-1 phosphorylation by incubation with the lymphocyte-specific protein tyrosine kinase Lck and/or CD45 in HEK293 cells in the presence or absence of CD45-PD1 bispecific diabody (see FIG. 5A).

As shown in FIG. 5B, cell surface-receptor PD-1 was not phosphorylated in wild-type HEK293 cells (lane 1). However, phosphorylation of PD-1 was readily observed when Lck was also present (lane 2). Co-expression of CD45 (lane 3) was observed to reduce overall phosphorylation. Upon incubation with a CD45-PD1 bispecific diabody (Db), a reduction in PD-1 phosphorylation was observed. In these experiments, approximately two million cells were transiently transfected with genes encoding full length human PD-1, Lck and CD45. After 24 hours, cells were either left untreated or incubated with the multivalent antibody CD45-PD1 constructed as described in Example 1 above for 15 min at room temperature, after which cells were lysed with lysis buffer (20 mM HEPES, 150 mM NaCl, 2 mM EDTA, 10% Glycerol, 2× Complete Protease Inhibitor Cocktail (Roche), 2 mM Sodium Orthovanadate (NEB), 1× Phosphatase Inhibitor Cocktail (Cell Signaling Technology), 1× DNAase (NEB), 1% NP-40) for 30 minutes on ice. After solubilization, the cell lysate was pre-cleared by centrifugation at 21,000g for 30 minutes at 4° C. PD-1 was then immunoprecipitated (IP) from the cell lysate with a biotinylated anti-PD1 antibody (Biolegend, Cat. No. 367418) coupled to streptavidin-coupled Dynabeads® (Thermo Fisher Scientific) for 1 hour on ice. After four washes with wash buffer (20 mM HEPES, 150 mM NaCl, 1% NP-40, 2× Complete Protease Inhibitor Cocktail (Roche), 2 mM Sodium Orthovanadate and 1× Phosphatase Inhibitor Cocktail), beads were incubated with non-reducing SDS sample buffer and heated at 95° C. for 5 minutes. After electrophoresis using SDS-PAGE, the IP samples were transferred to a PVDF membrane (Bio-Rad) and incubated with anti-Tyr phosphorylation (upper panel; Cell Signaling; P-Tyr-100, Cat. No. 9411S) or anti PD-1 (lower panel; Biolegend, Cat. No. 367402) antibodies for Western blotting assay (WB) as per manufacturer's instructions.

### Example 4

This Example describes experiments performed to illustrate a reconstitution of multiple receptor phosphorylation by incubation with the lymphocyte-specific protein tyrosine kinase Lck and/or CD45 in HEK293 cells. Since CD45 is a highly abundant phosphatase present in all lymphocytes, the experimental results described in this Example demonstrates that CD45 recruitment may be used to dephosphorylate multiple different receptors involved in different cellular function.

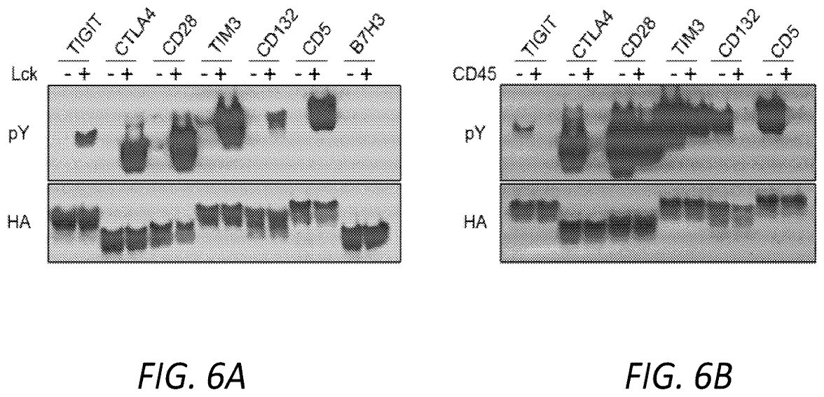

In these experiments, it was observed that CD45 could dephosphorylate multiple targets, indicating that CD45 activity is not specific to a particular target. TIGIT, CTLA-4, CD132, CD5 and to a lower extent CD28 and TIM-3 were all found to be dephosphorylated by CD45. As shown in FIG. 6A, cell surface-receptor TIGIT, CTLA4, CD28, TIM3, CD132, CD5 and B7H3 were not basally phosphorylated in wild-type HEK293 cells. Receptor phosphorylation was readily observed when Lck was also present, with the exception of B7H3 which does not contain a signaling Tyrosine residue (FIG. 6A). As shown in FIG. 6B, co-expression of CD45 was observed to reduce phosphorylation for all receptors tested, indicating that CD45 activity is not specific to a particular target. In these experiments, approximately two million cells were transiently transfected with genes encoding the intracellular region of human receptors, Lck and CD45. After 24 hours, cells were lysed with lysis buffer (20 mM HEPES, 150 mM NaCl, 2 mM EDTA, 10% Glycerol, 2× Complete Protease Inhibitor Cocktail (Roche), 2 mM Sodium Orthovanadate (NEB), 1× Phosphatase Inhibitor Cocktail (Cell Signaling Technology), 1× DNAase (NEB), 1% NP-40) for 30 minutes on ice. After solubilization, the cell lysate was pre-cleared by centrifugation at 21,000g for 30 minutes at 4° C. PD-1 was then immunoprecipitated (IP) from the cell lysate with anti-HA magnetic beads for 1 hour on ice. After four washes with wash buffer (20 mM HEPES, 150 mM NaCl, 1% NP-40, 2× Complete Protease Inhibitor Cocktail (Roche), 2 mM Sodium Orthovanadate and 1× Phosphatase Inhibitor Cocktail), beads were incubated with non-reducing SDS sample buffer and heated at 95° C. for 5 minutes. After electrophoresis using SDS-PAGE, the IP samples were transferred to a PVDF membrane (Bio-Rad) and incubated with anti-Tyr phosphorylation (upper panel; Cell Signaling; P-Tyr-100, Cat. No. 9411S) or anti PD-1 (lower panel; Biolegend, Cat. No. 367402) antibodies for Western blotting assay (WB) as per manufacturer's instructions.

### Example 5

This Example describes experiments performed to illustrate that treatment of T cells with a CD45-PD1 bispecific diabody increases T-cell activation in response to Muromonab-CD3 (OKT3) and peptide-MHC stimulation.

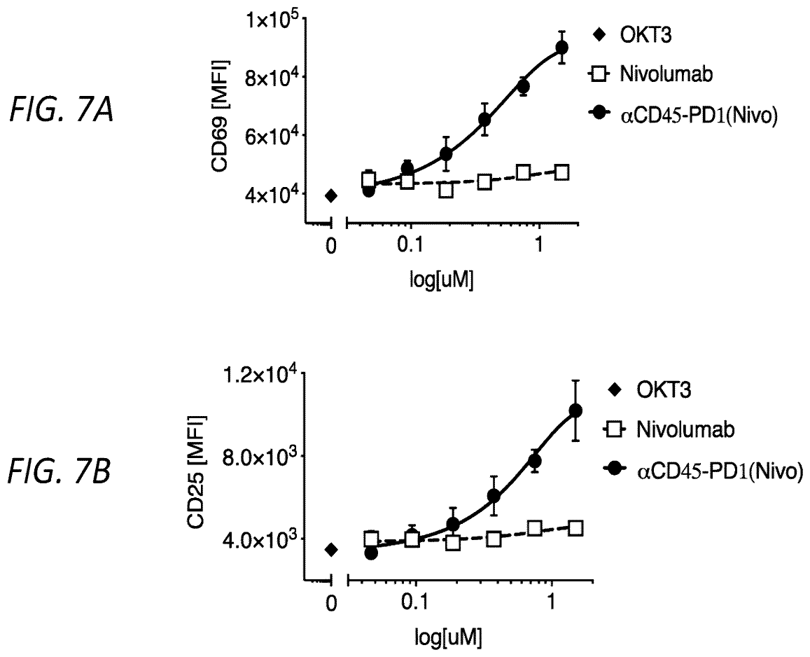

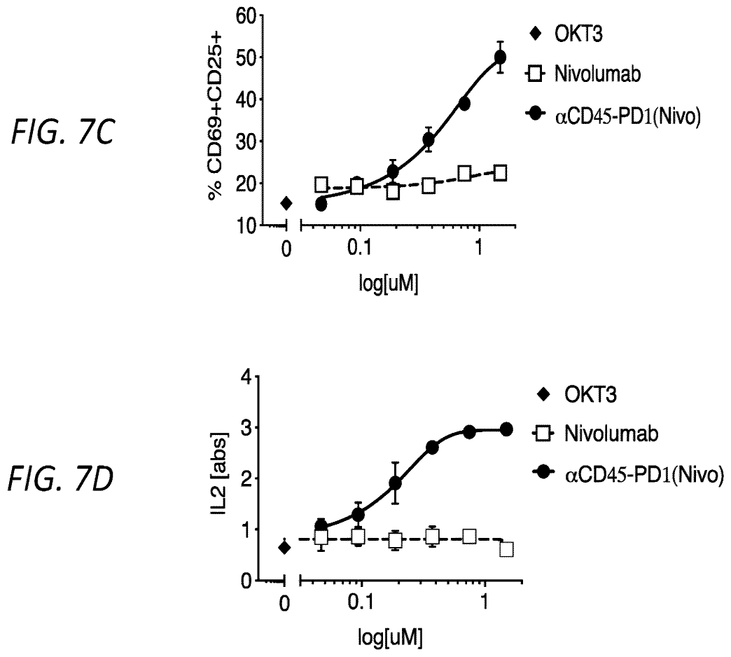

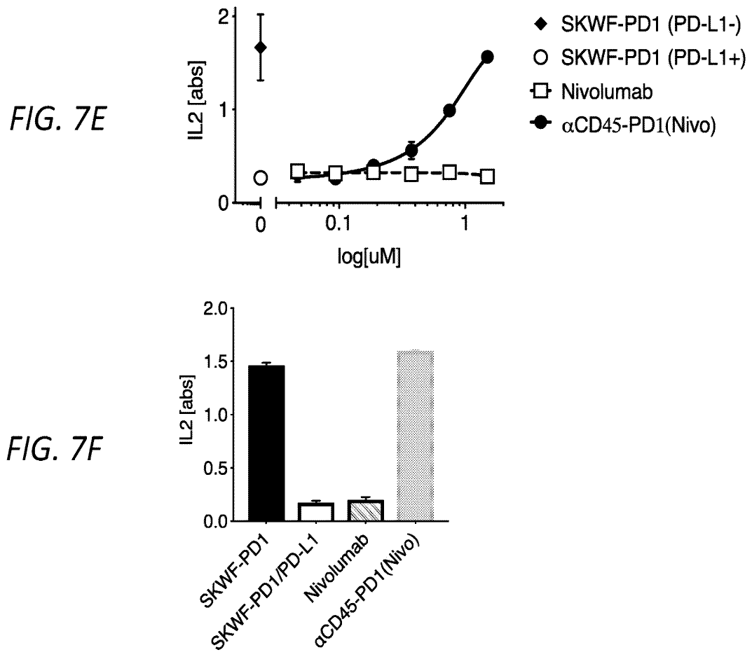

In these experiments, Jurkat T cells expressing PD-1 as described in Example 1 above (FIG. 3A) were stimulated with plate-bound OKT3 (2 μg/ml) alone (solid diamond) or nivolumab antibody (open square) or CD45-PD1 (Nivo) diabody (closed circle). It was observed that the bispecific diabody CD45-PD1 increased the expression of the activation markers CD69 (FIG. 7A) and CD25 (FIG. 7B-7C) as well as higher level of IL-2 cytokine secretion (FIG. 7D). Surface expression CD69 and CD25 was determined by FACS staining 16 hours following OKT3 stimulation. IL-2 concentration was quantified by ELISA (cat #431804, Biolegend) following Jurkat T-cell stimulation with OKT3(2 μg/ml) for 48 hours in the presence of nivolumab antibody (open square) or CD45-PD1 (Nivo) diabody (closed circle). Similar experiments were performed in a different T cell line, SKW-3 T cells (cat #ACC 53; DSMZ, Leibniz, Germany). As shown in FIGS. 7E-7F, SKW-3 T cells transduced with appropriate T-cell receptor (TCR) and PD-1 were incubated with cells presenting agonist peptide-MHC±PD-L1 for 48 hours (PD-L1-, solid diamond; PD-L1+, open circle) and nivolumab antibody (open square) or CD45-PD1 (Nivo) diabody (closed circle). Antigen presenting cells were incubated with 10 μM of agonist peptide for 1 hour at 37° C. prior to incubation with SKW-3 T cells. Surface expression of TCR, PD-1, MHC and PD-L1 was confirmed by FACS. It was found that incubation with the bispecific diabody CD45-PD1 described in Example 1 above increased IL-2 cytokine secretion to levels similar to those achieved when PD-L1 is absent. In FIGS. 7A-7F, representative data is shown as mean±SEM, n=3.

### Example 6

This Example describes experiments performed to illustrate that bispecific CD45-PD1 diabody can potentiate proliferation of activated peripheral blood mononuclear cells (PBMCs).

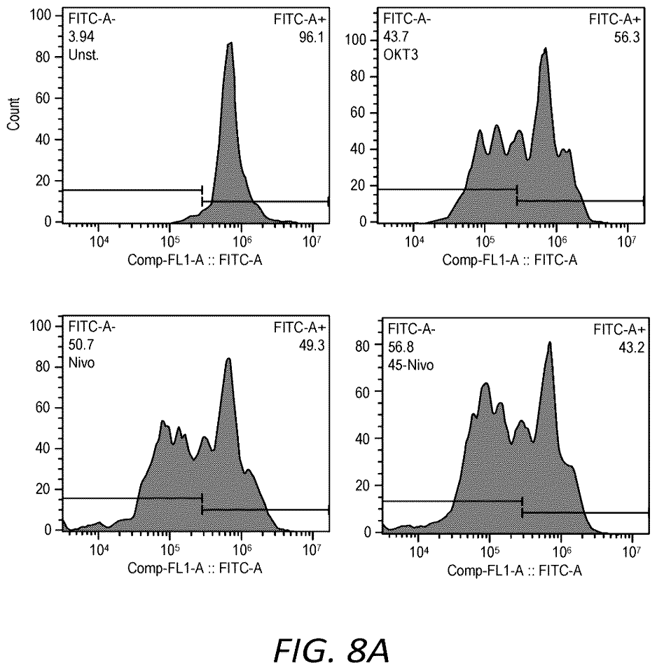

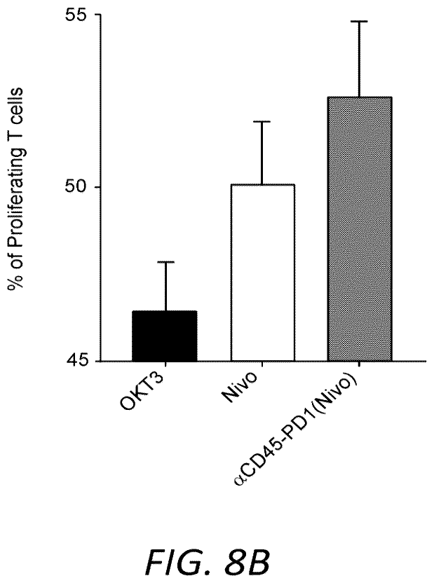

In these experiments, activated PBMC cells from healthy donors were isolated from leukapheresis chambers using standard Ficoll separation. PBMCs were rested in complete RPMI (10% FBS, 1×1 Glutamax, 1× Sodium Pyruvate, 1×HEPES and 1× Pen/Strep) overnight prior to the experiment. PBMCs at approximately 1 million/ml density were labeled with 1 μM CFSE for 10 minutes at room-temperature and incubated with plate-bound OKT3 at 1 μg/ml plus a commercial PD-1 antibody nivolumab or the bispecific CD45-PD1 diabody described in Example 1 above for 4 days. Data shown was gated on live T cells (CD3+/CD4+/CD8+), as determined by FACS staining. As shown in FIG. 8A, it was observed that CD45-PD1 potentiated T-cell proliferation to higher levels than nivolumab antibody.

In these experiments, CD45-PD1 and nivolumab were added at 0.5 μM final concentration. In addition, quantification of the percentage of proliferation for T cells for cells treated with OKT3 alone or OKT3 in combination with nivolumab or CD45-PD1 was also performed by FACS (FIG. 8B). Representative data shown as mean±SEM, n=3).

### Example 7

This Example describes another experiment performed with activated PBMCs to illustrate that bispecific diabody CD45-PD1 can potentiate CD4+ and CD8+ T-cell activation in response to agonist peptides and RIPR-PD1 is not strictly dependent on PD-1/PD-L1 interaction blockade.

PD-1 is known to reduce T-cell activity (also known as a “checkpoint inhibitor”), to do so PD-1 must be phosphorylated by an unknown mechanism, but which is assumed to be entirely dependent on binding to PD-L1. The inventors hypothesized that there are two components that contribute to PD-1 signaling: 1) Ligand binding and 2) tonic signaling (i.e., PD-1, or any other receptor, is expected to have low but functionally relevant, levels of phosphorylation even prior to ligand binding). Previously, there have been many efforts to control ligand-binding, including the development of blocking antibodies such as nivolumab or pembrolizumab. However, the contribution of tonic signaling has been largely overlooked. Without being bound to any particular theory, recruitment of CD45, a phosphatase, to PD-1 is expected to reduce PD-1 phosphorylation and therefore both tonic and ligand-induced PD-1 signaling. The outcome of compromised PD-1 activity was expected to be an enhanced T-cell activation. In this Example, the inventors showed that in freshly isolated lymphocytes, treatment with nivolumab and pembrolizumab antibodies, which blocks PD-1 binding to PD-L1, enhanced expression or secretion of various markers of activation, including CD69, CD25 and secreted cytokines such as IL-2 and IFNγ. In addition, treatment of these cells with RIPR using nivolumab or pembrolizumab arms to bind to PD—was shown to induce even higher levels of activation.

In these experiment, PBMCs, isolated as described in the previous Example 7, were first activated by incubation with a peptide pool composed of 176 peptides (JPT, PM-CEFX-1) at 50 μM (final concentration) for 24 hours, after which different antibodies or diabodies were added at 0.5 μM (final concentration). Tables 1 and 2 below provides a summary and brief description of diabody targets and compositions. The multivalent antibodies CD45-PD1 (Nivo) and CD45-PD-1 (Pembro) are described in SEQ ID NO: 12 and SEQ ID NO: 14 of the Sequence Listing (also see, Table 1)

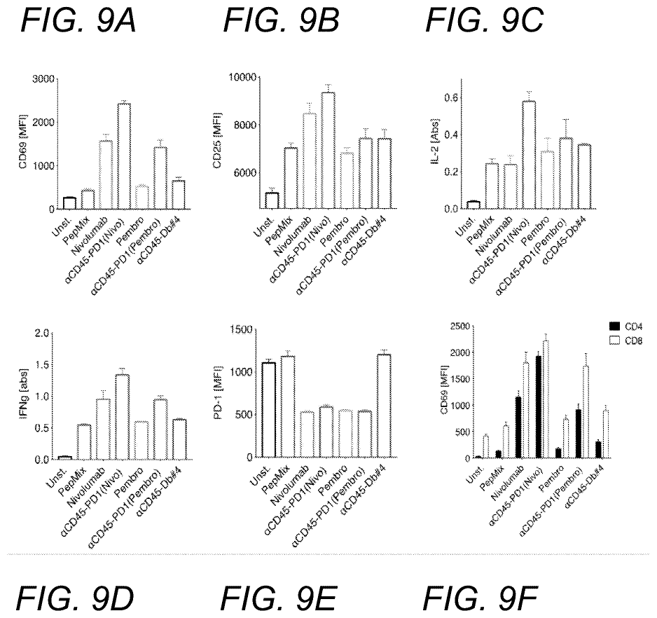

Treated cells and supernatants were harvested 24 hours after antibody or diabody treatment. It was observed that both CD45-PD1 (Nivo) and CD45-PD1 (Pembro) could potentiate T-cell activation as determined by elevated expression levels of CD69 (FIG. 9A) and CD25 (FIG. 9B) as determined by FACS (CytoFLEX), as well as secretion of IFNγ (FIG. 9D) and cytokine IL-2 (FIG. 9C), as determined by ELISA (IL-2 was quantified using cat #431804, Biolegend, and IFNγ was quantified using cat #430104, Biolegend, as per manufacturer's instructions). Data shown for CD69 was gated on live CD3+/CD4+/CD8+ cells. Summarized in FIG. 9E is a competition experiment where after treatment with nivolumab, pembrolizumab, or CD45-PD1 (Nivo) and CD45-PD1 (Pembro), T cells were stained with fluorescently labelled anti-PD1 blocking antibody, clone EH12.2H7 (Biolegend, Cat #329904). Clone EH12.2H7, nivolumab and pembrolizumab have overlapping epitopes, and thus the fluorescence intensity (PD-1 MFI) from Clone EH12.2H7 labelling was reduced after nivolumab or pembrolizumab treatment. RIPR-Nivo and RIPR-Pembro molecules also compromised clone EH12.2H7 labelling to similar extent, thus suggesting RIPR-Nivo and RIPR-Pembro maintained the PD-1 binding properties of nivolumab and pembrolizumab, respectively.

TABLE 1
Description of the RIPR binding modules, wild-type proteins and linker units

| | | | | | PD-1 | DNA | AA |
|---|---|---|---|---|---|---|---|
| Name | Format | Target 1 | Target 2 | Target 3 | Blocking | Sequence ID | sequence ID |
| CD45-PD1(Nivo)-v0.1 | Bispecific scFv | hCD45 | hPD-1 | NA | Yes | 9 | 10 |
| CD45-PD1(Nivo)-Stabilized* | Bispecific scFv | hCD45 | hPD-1 | NA | Yes | 11 | 12 |
| CD45-PD1(Pembro) | Bispecific scFv | hCD45 | hPD-1 | NA | Yes | 13 | 14 |
| CD45-PD1(CI19) | Bispecific scFV | hCD45 | hPD-1 | NA | No | 15 | 16 |
| CD45-Db-#4 | Diabody | hCD45 | hCD45 | NA | NA | 17 | 18 |
| CD45-PD1(VHH) | Bispecific scFv-VHH | hCD45 | hPD-1 | NA | Yes | 19 | 20 |
| CD45-PD1(VHH)-Extended | Bispecific scFv-VHH | hCD45 | hPD-1 | NA | Yes | 21 | 22 |
| CD45-PD1 (mRIPR-PD1) | Bispecific scFv-VHH | mCD45 | mPD-1 | NA | Yes | 23 | 24 |
| CD45-CTLA4 (mRIPR-CTLA4) | Bispecific VHH-VHH | mCD45 | mCTLA4 | NA | NA | 25 | 26 |
| CD45-PD1/CTLA4 (dRIPR-PD1/CTLA4) | Trispecific VHH-VHH-scFV | mCD45 | mPD-1 | mCTLA4 | Yes | 27 | 28 |
| Human PD-1 | NA | NA | NA | NA | NA | 29 | 30 |
| Mouse PD-1 | NA | NA | NA | NA | NA | 31 | 32 |
| Human CD45 | NA | NA | NA | NA | NA | 33 | 34 |
| Linker 1 (scFv-scFv) | NA | NA | NA | NA | NA | 35 | 36 |
| Linker 2 (scFv-scFv) | NA | NA | NA | NA | NA | 37 | 38 |
| Linker 3 (VHH-scFv; cleavable) | NA | NA | NA | NA | NA | 39 | 40 |
| Linker 4 (VHH-scFv)-Extended | NA | NA | NA | NA | NA | 41 | 42 |
| Linker 5 (VHH-scFv; non-cleavable) | NA | NA | NA | NA | NA | 43 | 44 |
| Linker 6 (scFv-scFv) | NA | NA | NA | NA | NA | 45 | 46 |
| Linker 7 (VHH-VHH) | NA | NA | NA | NA | NA | 47 | 48 |
| Linker 8 (VHH-VHH-scFv) | NA | NA | NA | NA | NA | 49 | 50 |
| Linker 9 (VHH-VHH-scFv) | NA | NA | NA | NA | NA | 51 | 52 |
| CD45-CD28 (mRIPR-CD28) | Bispecific scFv-VHH | mCD45 | mCD28 | NA | NA | 53 | 54 |

*Unless stated otherwise, CD45-PD1(Nivo)-Stabilized bispecific diabody is the default binding module and is also termed “CD45-PD1(Nivo)” in main text, figures and figure legends

### Example 8

This Example describes another set of experiments performed with activated PBMCs to illustrate that bispecific diabody CD45-PD1 (C119), using a non-blocking scFv to bind to PD-1 can potentiate T-cell activation in response to agonist peptides.

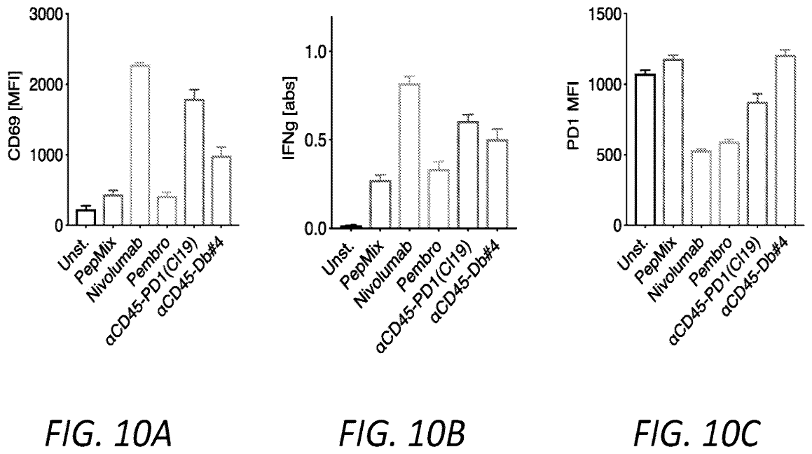

In this experiment, PBMCs, isolated as described in the previous Example 7, were first activated by incubation with a peptide pool composed of 176 peptides (JPT, PM-CEFX-1) at 50 M (final concentration) for 24 hours, after which different antibodies or diabodies were added at 0.5 μM (final concentration). Treated cells and supernatants were harvested 24 hours after antibody or diabody treatment. It was observed that CD45-PD1 (C119) could potentiate T-cell activation as determined by elevated expression levels of CD69 (FIG. 10A) as well as secretion of IFNγ (FIG. 10B) as determined by ELISA (IFNγ was quantified using cat #430104, Biolegend, as per manufacturer's instructions).

Data shown for CD69 and CD25 was gated on live CD3+/CD4+/CD8+ cells. Summarized in FIG. 10C is a competition experiment where after treatment with nivolumab, pembrolizumab or CD45-PD1 (C119), T cells were stained with fluorescently labelled anti-PD1 blocking antibody, clone EH12.2H7 (Biolegend, Cat #329904). Clone EH12.2H7, nivolumab and pembrolizumab have overlapping epitopes, and thus the fluorescence intensity (PD-1 MFI) from Clone EH12.2H7 labelling was lower when cells were treated with nivolumab or pembrolizumab. It was observed that 45-PD1 (C119) had a weaker effect in clone EH12.2H7 labeling (higher fluorescence, PD1 MFI) which suggests that CD45-PD1 (C119) epitope is different from nivolumab or pembrolizumab.

The experiments described in this Example shows that a hRIPR-PD1 molecule, which uses an anti-PD1 binding unit that does not fully block PD-1 binding to PD-L1, also promotes T-cell activity. Without being bound to any particular theory, the hRIPR-PD1 molecule, because it directly targets PD-1 phosphorylation was expected to reduce PD-1 signaling and thus enhanced T-cell activation even in the absence of PD-1/PD-L1 blockade. It was observed that αCD45-PD1 (C119) appeared to be weaker than nivolumab but stronger than pembrolizumab at potentiating T-cell activation.

### Example 9

This Example describes experiments performed to develop a third hRIPR-PD1 molecule that had a different architecture because it used a nanobody (single heavy chain) fused to a scFv. As discussed in greater detail below, this new RIPR molecule (2^{nd} generation) demonstrated increased purification yield and maintains binding to PD-1 and CD45, as determined by surface plasmon resonance (SPR)

The 2nd generation bispecific RIPR-PD1 molecule described in this Example used the same anti-CD45 scFv as described in Examples above but now fused to a nanobody (VHH) anti-human PD-1 (described in US20170137517A1). Accordingly, it was expected that this new αCD45-PD1 (VHH) bispecific molecule would bind to human CD45 and human PD-1.

This 2nd generation anti-human RIPR-PD1 (anti-CD45/anti-PD1) bispecific molecule composed of an anti-CD45 scFv bound to an anti-PD1 nanobody (VHH) was purified by size-exclusion (AKTA FPLC, GE Healthcare, Superdex 200 Increase; 280 nm absorbance). Protein integrity and purity were confirmed by non-reducing SDS-PAGE electrophoresis followed by standard Coomassie staining (data not shown). It was observed that this CD45-PD1 (VHH) was capable of binding to the extracellular region of human CD45 and human PD-1 proteins as determined by surface plasmon resonance technique (data not shown). The affinity (K_{D}) for CD45 was found to be approximately 700 nM and the affinity for PD-1 was found to be approximately 5 nM.

### Example 10

This Example describes experiments performed to illustrate that treatment of T cells with a second generation CD45-PD1 (VHH) bispecific binding module as described in Example 9 above could increase T-cell activation in response to Muromonab-CD3 (OKT3).

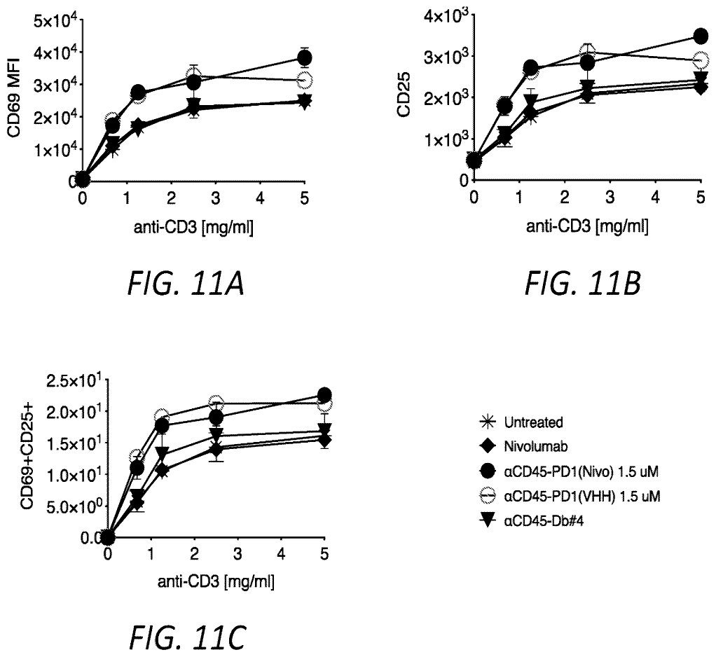

In these experiments, Jurkat T cells expressing were stimulated with plate-bound OKT3 at varying concentrations (from 0.625 to 5 μg/ml in the absence or presence of nivolumab (solid diamond), CD45-PD1 (Nivo) (closed circle), CD45-PD1 (VHH) (open circle), anti-CD45 diabody #4 (closed triangle) at 1.5 μM. It was observed that the bispecific diabody CD45-PD1 (Nivo) and CD45-PD1 (VHH) increased the expression of the activation markers CD69 (FIG. 11A) and CD25 (FIG. 11B) resulting in a higher fraction of CD69+/CD25+ cells (FIG. 11C). Surface expression CD69 and CD25 was determined by FACS staining 24 hours following OKT3 stimulation.

### Example 11

This Example describes experiments designed to develop a RIPR-PD1 molecule that targets mouse CD45 and mouse PD-1.

A mouse RIPR was constructed and composed of a nanobody (VHH) sequence targeting mouse CD45 directly fused to a scFv that recognizes mouse PD-1 (PD-1 scFv; PD1-F2, described previously in WO2004056875A1). Recombinant mRIPR was produced using the baculovirus-insect cell expression system in Trichoplusia ni (High Five™) cells. After Ni-NTA purification, mRIPR showed a momeric and monodisperse elution profile during the size-exclusion chromatography using 280 nm absorbance (data not shown). mRIPR purity was further confirmed by a non-reducing SDS-PAGE electrophoresis followed by standard Coomassie staining corresponding to peak fractions SEC elution (data not shown).

### Example 12

This Example describes experiments performed to illustrate that treatment of mouse T cells with an anti-mouse CD45 (VHH)-PDIF2 bispecific binding module increases T cell activation in response to anti mouse-CD3(2C11).

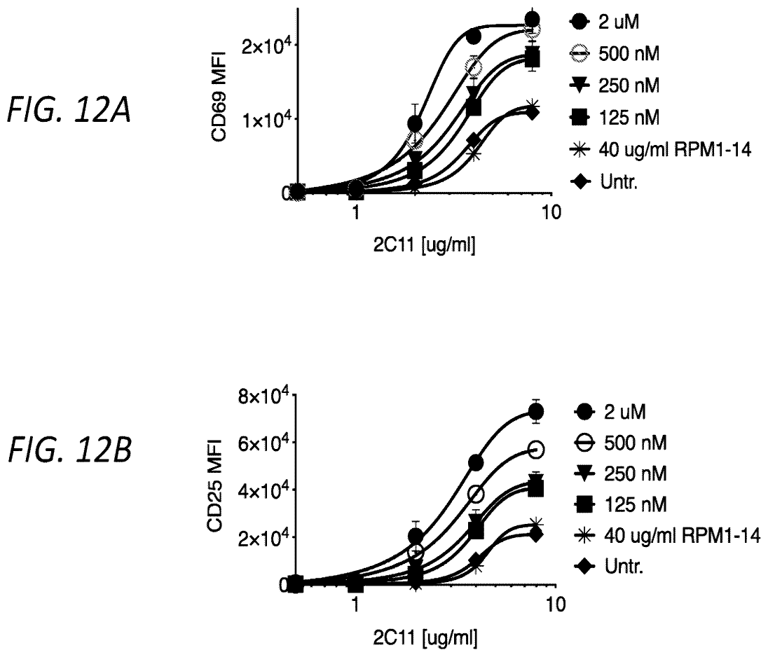

In these experiments, CD8+ T cells isolated from C57BL/6 mice were stimulated with plate-bound 2C11 at varying concentrations (from 1 to 10 g/ml) in the absence (Untreated; solid diamond) or presence of the CD45 (VHH)-PDIF2 bispecific binding module described in Example 11 above at varying concentrations. It was observed that the bispecific diabody CD45 (VHH)-PDIF2 increased the expression of the activation markers CD69 (FIG. 12A) and CD25 (FIG. 12B). In these experiments, surface expression CD69 and CD25 was determined by FACS staining 16 hours following 2C11 stimulation. The results of FACS analysis are summarized in Table 4 below, in which the percentage of double positive cells (CD69+CD25+) in each cohort is shown.

TABLE 4

| Concentration | Untreated | αPD-1 (RMP1-14) | mRIPR-PD1 |
|---|---|---|---|
| 4 μg/ml αCD3ε | 28.3 | 28.3 | 62.9 |
| 8 μg/ml αCD3ε | 44.6 | 56.6 | 82.9 |

### Example 13

This Example describes experiments performed to illustrate that treatment of mouse TCR transgenic (Pmel-1) CD8+ T cells with an anti-mouse CD45 (VHH)-PD1 (F2) bispecific binding module increases T-cell activation in response to gp100 peptide.

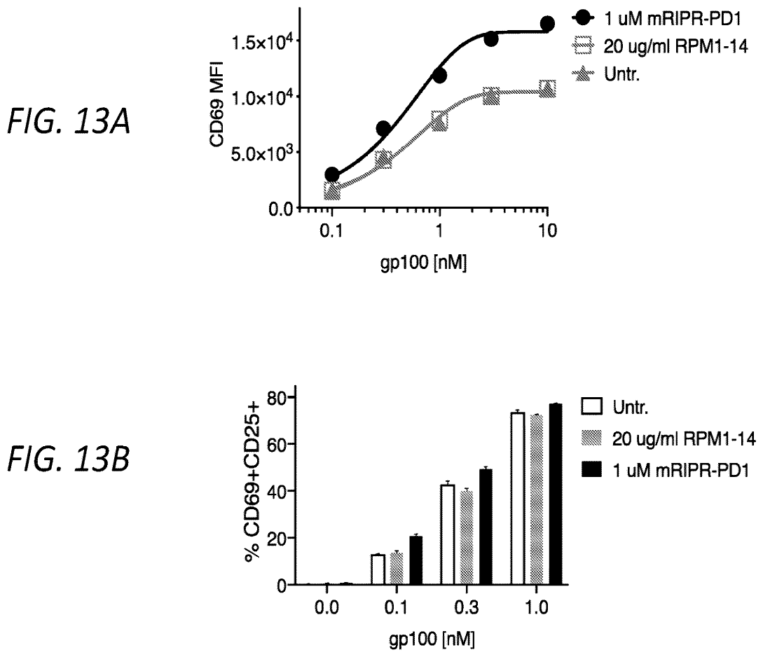

In these experiments, mouse CD8+ T cells expressing the Pmel-1 TCR were incubated with total splenocytes at 1:1 ratio and were stimulated with gp100 peptide (from 0.1 to 10 μM) in the absence (Untreated) or presence of CD45 (VHH)-PD1 (F2), or anti-PD1 blocking antibody RMP-14 at 1 μM. It was observed that the bispecific CD45 (VHH)-PD1F2 binding molecule increased the expression of the activation markers CD69 (FIG. 13A) and CD25 (FIG. 13B). Surface expression CD69 and CD25 was determined by FACS staining 24 hours following gp100 peptide stimulation. The results of FACS analysis are summarized in Table 5 below, in which the percentage of double positive cells (CD69+CD25+) in each cohort is shown.

TABLE 5

| Concentration | Untreated | αPD-1 | mRIPR-PD1 |
|---|---|---|---|
| 1 nM gp100 | 71.2 | 72.2 | 77.4 |
| 300 pM gp100 | 39.9 | 39.1 | 50.1 |
| 100 pM gp100 | 13.0 | 13.1 | 21.4 |

### Example 14

This Example describes experiments performed to illustrate that treatment of mouse T cells with mRIPR-CTLA4, an anti-mouse CD45 (VHH)-CTLA4 bispecific binding module, increases T-cell activation in response to anti mouse-CD3(2C11).

CTLA-4, as PD-1, reduces T-cell activity. The inventors developed a RIPR molecule that recruits CD45 to CTLA4. As for PD-1, recruitment of CD45 activity was expected to reduce CTLA-4 phosphorylation and because CTLA-4 is an inhibitor of T-cell activity, RIPR-CTLA4 is predicted to enhance T-cell function.

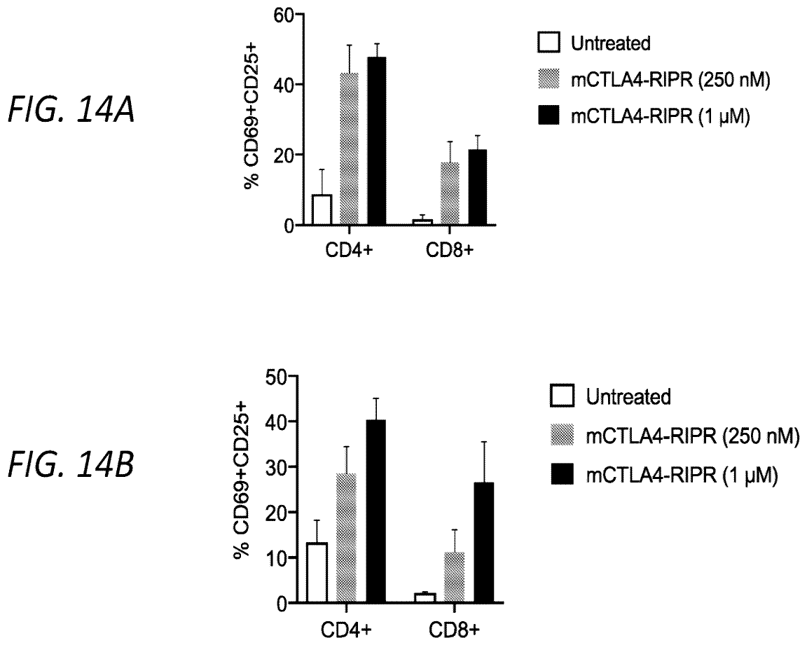

In these experiments, T cells isolated from C57BL/6 mice were stimulated with plate-bound 2C11 at 1 μg/ml in the absence or presence of mRIPR-CTLA4 at 250 nM or 1 μM. It was observed that the mRIPR-CTLA4 increased the expression of the activation markers CD69 and CD25, leading to an increase in the fraction of CD69+/CD25+ cells for both CD4+ and CD8+ 24 hours (FIG. 14A) and 48 hours (FIG. 14B) after incubation with 2C11 antibody. Surface expression CD69 and CD25 was determined by FACS staining at the indicated time points after 2C11 stimulation. Data shown was gated on live CD3+/CD4+ or CD3+/CD8+ T cells.

### Example 15

This Example describes experiments performed to illustrate that treatment of mouse T cells with an anti-mouse CD45 (VHH)-CTLA4 bispecific binding module, mRIPR-CTLA4, increases T-cell activation in response to anti mouse-CD3(2C11).

In these experiments, T cells isolated from C57BL/6 mice were stimulated with plate-bound 2C11 at 1 μg/ml in the absence (left panels) or presence of CD45 (VHH)-mCTLA4 (right panels) at 1 μM. It was observed that the bispecific diabody CD45 (VHH)-CTLA4 increased the expression of the activation markers CD69 and CD25, leading to an increase in the fraction of CD69+/CD25+ cells for both CD4+ and CD8+ 24 hours and 48 hours after incubation with 2C11 antibody. Surface expression CD69 and CD25 was determined by FACS staining at appropriate time points after 2C11 stimulation. Data shown in Table 4 was gated on live CD3+/CD4+ or CD3+/CD8+ T cells. The data shown in Table 6 is an example of the CD25 and CD69 surface staining corresponding to an activation with plate-bound 2C11 at 1 μg/ml and mRIPR-CTLA4 at 1 μM as described in Example 14 above.

TABLE 6

| Time after | CD4+ | | CD8+ | |
|---|---|---|---|---|
| incubation | | +CTLA4 RIPR | | +CTLA4 RIPR |
| with 2C11 | Untreated | (1 μM) | Untreated | (1 μM) |
| 24 hours | 13.7 | 50.5 | 9.87 | 45.1 |
| 48 hours | 2.53 | 24.3 | 2.02 | 33.5 |

### Example 16

This Example describes experiments performed to illustrate that treatment of mouse T cells with mRIPR-CD28, an anti-mouse CD45 (VHH)-CD28 bispecific binding module, mRIPR-CD28 reduces the expression of markers of T-cell activation, such as CD25 and CD44, in response to anti mouse-CD3(2C11).

As discussed above, CD28 is part of the same protein family as PD-1 and CTLA-4, the B7 family of cell surface co-receptors. Contrary to PD-1 and CTLA-4, signaling by the CD28 co-receptor potentiates T-cell activation. With being bound to any particular theory, the recruitment of a phosphatase, such as CD45, to CD28 is expected to impair CD28 signaling and reduce (e.g., suppress) T-cell activation.

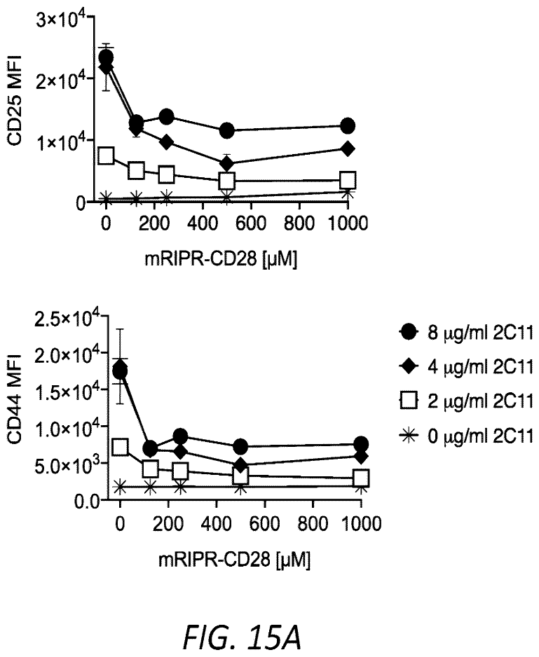

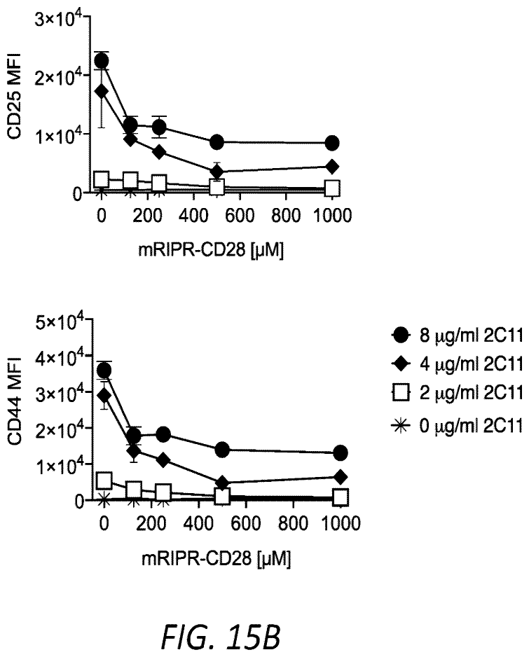

The mRIPR-CD28 used in these experiments included a nanobody anti-mouse CD28 (WO2002047721A1) fused to a nanobody anti-CD45 (PMID: 25819371). T cells isolated from C57BL/6 mice were stimulated with plate-bound 2C11 at 0.5, 1, 2, 4 or 8 g/ml in the absence or presence of mRIPR-CD28 at 125, 250, 500 or 1000 M. It was observed that the mRIPR-CD28 reduces the expression of the activation markers CD25 and CD44, for both CD4+ (FIG. 15A) and CD8+ (FIG. 15B) T cells after incubation with 2C11 antibody and mRIPR-CD28 for 48 hours. Surface expression CD25 and CD44 was determined by FACS staining at the indicated time points after 2C11 stimulation. Data shown was gated on live CD4+ or CD8+ T cells.

### Example 17

This Example describes a trispecific version of the RIPR molecule which was designed to recruit CD45 to two different cell surface antigens, PD-1 and CTLA4, and designated double RIPR (dRIPR)-PD1/CTLA4). This trispecific version of the RIPR molecule binds to mouse CD45, PD-1, and CTLA4 and is expected to potentiate T-cell activation.

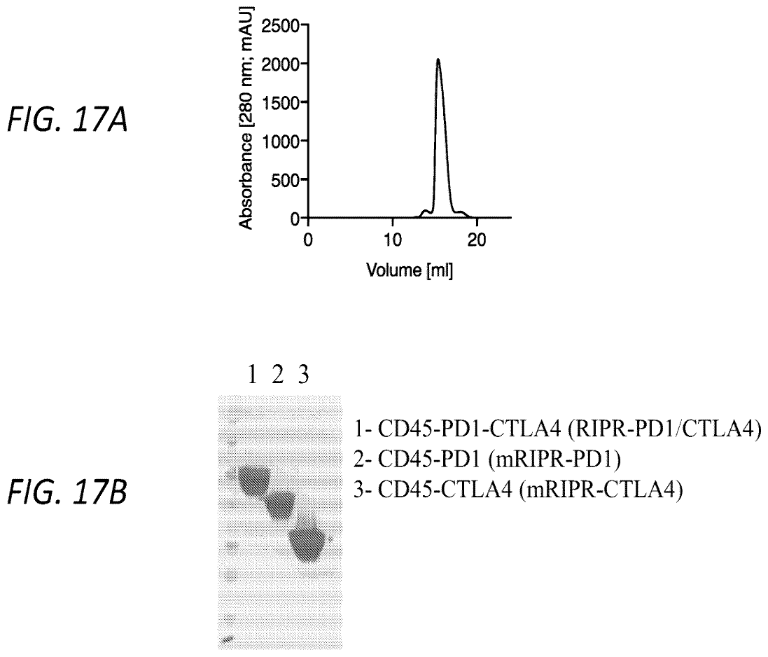

This molecule is composed of a nanobody anti-CTLA4 (PMID: 29581255) fused to a nanobody anti-CD45 (PMID: 25819371) and a scFv anti-PD1 (PD1-F2, described in WO2004056875A1). The amino acid sequence of the dRIPR-PD1/CTLA4 is set forth in SEQ ID NO: 28 of the Sequence Listing. Further information regarding dRIPR-PD1/CTLA4 can also be found in Table 1. This anti-mouse trispecific CD45-PD1-CTLA4 was subsequently purified by size-exclusion (AKTA FPLC, GE Healthcare, Superdex 200 Increase; 280 nm absorbance is shown in FIG. 17A). In addition, protein integrity and purity of the trispecific CD45-PD1-CTLA4 molecule were confirmed by non-reducing SDS-PAGE electrophoresis followed by standard Coomassie staining (FIG. 17B).

### Example 18

This Example describes experiments performed to demonstrate that a multivalent polypeptide including an anti-CD45 scFv fused to a cytokine, in this case interleukin-2, decreases phosphorylation of STAT5 (pSTAT5) and reduces STAT5 signaling.

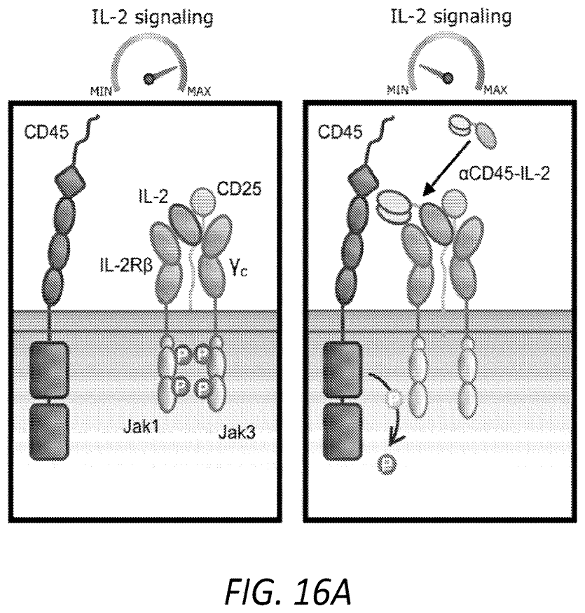

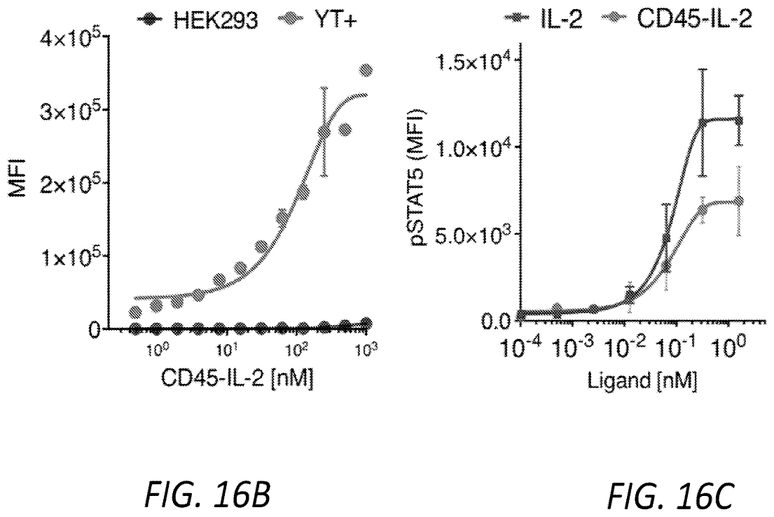

In these experiments, in order to recruit the CD45 phosphatase to the IL-2R, the anti-human CD45 scFv was fused to wild-type IL-2 as follows: a multivalent polypeptide capable of binding to CD45 and IL-2R was constructed, wherein the amino acid sequence of the polypeptide includes, in N-terminal to C-terminal direction: (i) a heavy chain variable region of a scFv specific for CD45, (ii) a light chain variable region of the scFv specific for CD45; and (iii) an amino acid sequence for cytokine IL-2 having a binding affinity for the cytokine receptor IL-2R. The amino acid sequence of the multivalent polypeptide antiCD45-IL2 is disclosed in SEQ ID NO: 6 of the Sequence Listing. As summarized in FIG. 16A, it is believed that IL-2 induces JAK Tyr phosphorylation upon binding to the IL-2 receptor, and that local phosphatase recruitment of CD45 to the cytokine receptor IL-2R decrease phosphorylation of STAT5 (pSTAT5).

In these experiments, surface staining of HEK293s (grey) and YT+ cells (CD25+; red) with fluorescently labeled antiCD45-IL-2 multivalent polypeptide (labelled with Alexa Fluor647 as per manufacturer's instructions; Thermo Fisher Scientific) is shown in FIG. 16B. Also, as shown in FIG. 16C, incubation of YT+ cells with antiCD45-IL2 multivalent polypeptide at the indicated concentrations for 15 minutes at 37° C. leads to a ˜50% decrease in pSTAT5 Emax as compared to wild-type IL-2. Representative data is shown as mean±SD, n=3. To determine the extent of pSTAT5 in response to IL-2 or CD45-IL2 chimera, approximately 100,000 cells were fixed in 4% PFA for 10 minutes at room temperature. Following fixation, cells were permeabilized with methanol on ice for 1 hour followed by an overnight incubation at −80° C. After washing with MACS buffer (Miltenyi), cells were incubated with fluorescently labelled anti-human pSTAT5 antibody at a 1:100 dilution in MACS buffer (AlexaFluor® 647; BD Biosciences cat #612599) for 1 hour on ice. After three washes in ice-cold MACS buffer, pSTAT5 was quantified by FACS (CytoFLEX).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1.-81. (canceled)

82. A multivalent polypeptide comprising:
a first amino acid sequence comprising a first polypeptide module capable of binding to one or more receptor protein-tyrosine phosphatases (RPTP); and
a second amino acid sequence comprising a second polypeptide module capable of binding to one or more cell surface receptors that signal through a phosphorylation mechanism;
wherein the first polypeptide module is operably linked to the second polypeptide module.

83. The multivalent polypeptide of claim 82, wherein the first polypeptide module is operably linked to the second polypeptide module via a polypeptide linker sequence.

84. The multivalent polypeptide of claim 82, wherein at least one of the first and second polypeptide modules comprises an amino acid sequence for a protein-binding ligand or an antigen-binding moiety.

85. The multivalent polypeptide of claim 84, wherein the antigen-binding moiety is selected from the group consisting of a single-chain variable fragment (scFv), an antigen-binding fragment (Fab), a nanobody, a VH domain, a VL domain, a single domain antibody (dAb), a VNAR domain, and a VHH domain, a diabody, or a functional fragment of any thereof.

86. The multivalent polypeptide of claim 85, wherein the antigen-binding moiety comprises a heavy chain variable region (VH) and a light chain variable region (VL).

87. The multivalent polypeptide of claim 84, wherein the protein-binding ligand is a cytokine, a growth factor, a receptor extracellular domain (ECD) of a cell surface receptor or of a RPTP, or a functional variant of any thereof.

88. The multivalent polypeptide of claim 82, wherein the one or more RPTPs comprise CD45 or a functional variant thereof.

89. The multivalent polypeptide of claim 82, wherein the one or more cell surface receptors comprise an immune-checkpoint receptor, a cytokine receptor, or a growth factor receptor.

90. The multivalent polypeptide of claim 86, wherein the VH and the VL of the antigen-binding moiety are operably linked to each other via one or more intervening amino acid residues that are positioned between the VH and the VL.

91. The multivalent polypeptide of claim 82, comprising, in the N-terminal to C-terminal direction:
a) a domain A comprising a binding region of a VH of a first scFv specific for an epitope of a RPTP;
b) a domain B comprising a binding region of a VL of a second scFv specific for an epitope of a cell surface receptor;
c) a domain C comprising a binding region of a VH of the second scFv specific for an epitope of the cell surface receptor; and
d) a domain D comprising a binding region of a VL of the first scFv specific for an epitope of the RPTP.

92. The multivalent polypeptide of claim 82, further comprising an amino acid sequence for a signal peptide.

93. The multivalent polypeptide of claim 82, wherein the multivalent polypeptide comprises an amino acid sequence having at least 80% sequence identity to an amino acid sequence selected from the group consisting of SEQ ID NOS: 2, 4, 6, 10, 12, 14, 16, 20, 22, 24, 26, 28, and 54.

94. A pharmaceutical composition comprising a multivalent polypeptide according to claim 82, and a pharmaceutical acceptable excipient.

95. A recombinant nucleic acid molecule comprising a nucleotide sequence encoding a polypeptide that comprises an amino acid sequence having at least 80% identity to the amino acid sequence of the multivalent polypeptide of claim 82.

96. An expression cassette or a vector comprising the recombinant nucleic acid molecule of claim 95.

97. A recombinant cell comprising: (a) a multivalent polypeptide according to claim 82 and/or (b) a recombinant nucleic acid molecule encoding the multivalent polypeptide of (a).

98. A cell culture comprising one or more recombinant cells of claim 97 and a culture medium.

99. A method for producing a polypeptide comprising:
providing one or more recombinant cells of claim 97; and
culturing the one or more recombinant cells in a culture medium such that the cells produce the multivalent polypeptide encoded by the recombinant nucleic acid molecule.

100. A method for modulating cell signaling mediated by a cell surface receptor that signals through a phosphorylation mechanism in a subject, the method comprising administering to the subject a first therapy comprising an effective amount of
a) a multivalent polypeptide according to any one of claim 82;
b) a recombinant nucleic acid comprising a nucleotide sequence encoding a polypeptide encoding the multivalent polypeptide of (a); and/or
c) a recombinant cell comprising the multivalent polypeptide of (a) and/or the recombinant nucleic acid of (b); and/or d) a pharmaceutical composition comprising the multivalent polypeptide of (a), the recombinant nucleic acid of (b); and/or the recombinant cell of (c).

101. A method for the treatment of a disease in a subject in need thereof, the method comprising administering to the subject a first therapy comprising an effective amount of
(a) a multivalent polypeptide according to claim 82;
(b) a recombinant nucleic acid comprising a nucleotide sequence encoding a polypeptide encoding the multivalent polypeptide of (a); and/or
(c) a recombinant cell comprising the multivalent polypeptide of (a) and/or the recombinant nucleic acid of (b); and/or d) a pharmaceutical composition comprising the multivalent polypeptide of (a), the recombinant nucleic acid of (b); and/or the recombinant cell of (c)
